(19) United States
(12) Patent Application Publication
(10) Pub. No.: US 2024/0290476 A1
(43) Pub. Date: Aug. 29, 2024

(54) METHOD AND SYSTEM FOR COMPUTER-AIDED TRACKING OF A PROCEDURE TO BE CARRIED OUT BY A PERSON

(71) Applicants: PULVIS BETEILIGUNGS GMBH, Altomünster (DE); GILL BETEILIGUNGS GMBH, Holzgerlingen (DE)

(72) Inventors: Jürgen STAUBER, Altomünster (DE); Bernd GILL, Holzgerlingen (DE)

(21) Appl. No.: 18/570,397

(22) PCT Filed: Jun. 15, 2022
(86) PCT No.: PCT/EP2022/066359

§ 371 Date: Dec. 14, 2023

(30) Foreign Application Priority Data
Jun. 15, 2021 (EP) 21179652.9

(51) Int. Cl. G16H 40/20; G16H 10/40; G16H 40/67

(52) U.S. Cl. CPC G16H 40/20; G16H 10/40; G16H 40/67

(57) ABSTRACT

The disclosure relates to a method and a system for computer-aided tracking of a procedure to be carried out by a person, in particular a production procedure or test procedure, for example for a virus detection test, preferably for carrying out an antigen test for SARS-CoV-2. The disclosure also relates to a method and system for computer-aided validation of a procedure carried out by a person and tracked by computer. The disclosure also relates to a method and system for setting up a person profile for use in one of the aforementioned methods and systems. The disclosure also relates to a computer program product.

### TECHNICAL FIELD OF THE INVENTION

The invention relates to a computer-implemented method and a system for computer-aided tracking of a procedure to be carried out by a person, in particular a production procedure or test procedure, for example for a virus detection test, preferably for carrying out a SARS-CoV-2 test. The invention also relates to a method and system for computer-aided validation of a procedure carried out by a person and tracked by a computer. The invention also relates to a method and system for setting up a person profile for use in one of the aforementioned methods and systems. The invention also relates to a computer program product.

### TECHNICAL BACKGROUND

A procedure can be carried out by a person manually and/or using aids. The procedure can be performed on the person themselves, an animal or an object. Proper performance of the procedure determines whether the procedure has been carried out with the desired success. The procedure can be safeguarded by validation, quality assurance and/or certification (hereinafter collectively referred to as validation) in order to recognize attempts at manipulation or to detect errors during the performance of the procedure.

For this purpose, the procedure to be carried out can be randomly checked by another person (=supervisor) while the procedure is being carried out.

The disadvantage that the other person must be physically present in order to follow the procedure during its execution can now be circumvented by the other person being able to observe the execution of a procedure remotely by video communication and thus recognize manipulations and errors during the execution of the procedure and validate or not validate the procedure accordingly.

For example, WO 2014/114 513 A1 describes this type of “remote observation” for identifying a person. Here—as an alternative to a classic post-ident procedure—video communication is started to a remote server and the person to be identified is asked to position themselves in front of a terminal device with a camera using an identification document. The person is instructed to hold the identification document up to the camera and turn it at certain times. The person is also instructed to hold a face up to the camera and turn it if necessary. The other person compares the photograph of the identification document with the person's face in front of the terminal device and, if necessary, requests data from the identification document in order to verify the person's identity.

Even this remote observation does not solve the problem that the other person must be available to follow up a procedure. The availability of such an additional person may be limited, resulting in waiting times when validating the procedure. Keeping an additional person available therefore increases personnel costs and severely restricts the validation process in terms of time.

In addition, this other person may overlook errors when carrying out the procedure, so that the validation either fails unjustifiably (false negative validation) or is carried out unjustifiably (false positive validation).

### SUMMARY OF THE INVENTION

The invention is based on the problem of creating a method and system for tracking a procedure to be carried out by a person in order to avoid errors more reliably during the execution of the procedure and in which errors are reliably recognized during the execution, wherein the presence of another person is to be completely dispensed with. Tracking should be possible in real time.

The problem is solved by the features described in the independent claims. Advantageous embodiments of the invention are given in the dependent claims.

According to the invention, a computer-implemented method for computer-aided tracking of a procedure to be performed by a person is proposed. The method comprises the step of: identifying the person immediately before the person performs the procedure by capturing biometric data of the person by a terminal device; and comparing the captured biometric data with biometric data of a personal data set by the terminal device, wherein the personal data set is stored in a storage unit of the terminal device; identifying an object to be modified by the procedure by the terminal device immediately prior to performing the procedure; tracking a position of the person and the at least one object by the terminal device using methods of computer-aided gesture and/or pattern and/or image recognition during the performance of the procedure; instructing the person to perform the procedure by the terminal device during the performance of the procedure.

In one embodiment, documentation of the performance of the procedure is provided by capturing images at one or more random times during the performance of the procedure using a camera of the terminal device and storing this procedure documentation locally in the terminal device (30) in a procedure identification data set in the method.

The problem is also solved by a system for computer-aided tracking of a procedure to be carried out by a person. The system comprises an identification unit of a terminal device, which is set up to identify the person immediately before the person performs the procedure. The identification unit comprises an capturing unit of the terminal device, adapted to capture biometric data of the person; and an evaluation unit of the terminal device, adapted to compare the acquired biometric data with biometric data of a person data set, wherein the person data set is stored in a storage unit of the terminal device; wherein the identification unit is further adapted to identify an object to be changed by the procedure immediately before the procedure is performed; a tracking unit of the terminal device adapted to track at least a position of the person and at least a position of the object by the terminal device by methods of computer-aided gesture and/or pattern and/or image recognition during the performance of the procedure; and a display unit of the terminal device adapted to instruct the person to perform the procedure during the performance of the procedure.

In one embodiment of the system, the performance of the procedure is documented by capturing images at one or more random times during the performance of the procedure by a camera of the terminal device and this procedure documentation is stored locally in the terminal device in a procedure identification data set.

All process steps are carried out by one (the same) terminal device. All units are units of the (same) terminal device. A terminal device can be an electronic terminal device comprising a display unit (display), an input unit (touchscreen, keyboard, control panel), a recording unit (camera, barcode scanner) and an evaluation unit (control unit, CPU). The terminal device is, for example, a PC, a laptop, a smartphone, a tablet, a terminal and/or another electronic device. The terminal device is set up to carry out methods of computer-aided gesture and/or pattern and/or image recognition.

A central criterion of the test procedure application on the terminal device is the protection of personal data. The test procedure application ensures that all personal data is only stored in encrypted form in the test procedure application on the person's terminal device.

In order to make the monitored and certified procedure tamper-proof, the entire data set is divided into (1) data stored in the test procedure application, in particular personal data, and (2) centrally stored data, in particular non-personal data.

This architecture is based on three core components:
(1) Personal data, such as name, date of birth, address, document number, document type, profile picture, documentation pictures of all test procedures performed together with a biometric profile.

The advantage is that the biometric profile is used to access this personal data or to add new personal data, for example to create a new test or to access the test certificates. Without the identification of the person, all this data remains hidden from other users.
(2) A scatter value (e.g. hash value) of the entire personal profile data set is used as a cryptographic key between the personal data from (1) on the terminal device and the centrally stored data, in particular the non-personal data. This hash value makes it impossible to trace a person, as the hash value is only based on data fragments of the personal data. Even if the same person were to recreate the profile, the hash value would change if a new profile picture is taken that differs from the first profile picture.

In addition, the hash value key is implemented unidirectionally so that the test procedure application can retrieve and write centrally stored non-personal data. In contrast, there is no application programming interface, API, via which the test procedure application can access personal data via the hash value key.

The advantage is that it ensures that if a user profile is deleted, there is no possibility of tracing the user back to the centrally stored (non-personal) data under the first profile. This also prevents forensic reconstruction of the original data sets without having to delete the non-personal data.
(3) The non-personal data is stored centrally in the background system and linked to the personal data in (1) using the hash value key from (2). Non-personal data includes, for example, anonymous users, the number of failed test procedures and all test-related data from tests carried out, such as the type of test manufacturer, test time, test date, test location (at postcode level) and test result. The hash value key allows all decentrally stored data to be assigned to an anonymous user—which makes it possible to generate real-time statistics on the overall process.

These statistics can be used to either monitor the overall usage of the process, for example statistics provided to health authorities that provide real-time information on where testing procedures are being performed and measuring a rate of positive test results to identify infection hotspots, but also to monitor the use of the test procedure application and prevent misuse, for example if a user attempts to cheat or fails the test procedure application several times, this behavior is used to identify users who are clearly not using the test procedure application with the right intention. Access can then be blocked centrally without having to actually know the user. In addition, user friendliness can be recorded and the use of the test procedure application can be monitored across all users in order to understand in which steps there are the most manageability problems and what these are based on. Long-term statistics, such as the development of test behavior and the positive rate of one region compared to another, can also be recorded and evaluated.

The advantage is the use of centrally stored data in a background system for statistical purposes for monitoring and to improve user-friendliness, without a single record being traced back to an individual user and without this data being lost if a user requests or initiates the deletion of their personal data.

The overall benefit of this architecture based on three core components is the protection of personal data and only the personal data on the terminal device and the centrally stored non-personal data in the background system, which are linked via the hash value key and can be retrieved by the test procedure application, offer the authorized user the full benefit after successful biometric identification.

One or more sensors can be connected to the terminal device. One or more sensors, in particular acceleration or position sensors, can be arranged on the person, for example on an extremity, in particular one or both hands. One or more sensors, in particular acceleration or position sensors, can provide one or more movement and/or position information about the at least one position of the person to the terminal device. One or more sensors can be arranged on one or more objects, for example one sensor per object. This one or more sensors can provide one or more movement and/or position information about the at least one position of the at least one object to the terminal device. These signals are preferably provided wirelessly, for example by Bluetooth communication and/or NFC communication.

Alternatively or additionally, an optical marking can be attached to the person and or the at least one object, which facilitates (enables) a detection unit to track the at least one position of the person and the at least one position of the at least one object.

The evaluation unit of the terminal device is set up to instruct the person by the display unit in such a way that the person is guided through the test procedure with detailed and step-by-step instructions. Computer-aided artificial intelligence (AI) and augmented reality (AR) methods are used in the system to track the test procedure itself, the movement sequences and objects, and possibly also aids, in order to ensure that the test is carried out correctly. In addition, the evaluation unit of the terminal device is set up to randomly capture images of the person or the objects or aids during the procedure and store them in a procedure documentation in a storage unit of the terminal device. The person can be identified on the images beforehand.

Each of the instructions can contain an animated video. The instruction can be supported by augmented reality. During a process step, the process step can be documented by taking at least one picture of the person during the process step using a camera of the terminal device. Several, for example two, images can also be taken by the camera of the terminal device of the person during the process step. One or more videos can also be taken by the camera of the terminal device of the person during the process step. These recordings are stored in a product documentation in the data set in the storage unit of the terminal device.

Each process step of the test procedure can be tracked by the terminal device. Depending on the process, the step is checked to see whether it was successfully carried out by the same test person in accordance with the instructions.

The person can be informed that a procedure has failed due to violation of the procedure instructions or due to cancellation.

The evaluation unit of the terminal device is set up to check whether a process step that has just been tracked is the last process step of the procedure. If the check shows that the process step that has just been tracked is not the last process step of the procedure, a further (next) process step is instructed via the display unit of the terminal device.

Preferably, the terminal device is the user's smartphone. The smartphone can be easily transported and may already be sufficiently personalized.

A procedure can be a sequence of steps to achieve a result. The sequence can be fixed, so that sequences of steps must be observed. Alternatively, the sequence can be arbitrary and only the result of the procedure is decisive.

A procedure can be a test procedure, for example a human medical test procedure.

The procedure may be a veterinary test procedure for the examination of a body fluid on an animal. The veterinary test procedure is performed by a person.

The test procedure may be an examination of a body fluid. The test procedure may be for testing a body fluid of a person and/or an animal for infections, characteristics and/or parameters. For example, the test procedure is the performance of a virus detection test, a germ test, a bacterial test and/or a fungal test on a person or an animal.

The body fluid can be the blood, swab, saliva, aerosol, tear fluid, sweat, sputum, urine and/or feces of a person and/or an animal. The body fluid can be taken from a skin surface, for example from a hand or a forehead.

The procedure can be the removal of the body fluid sample immediately followed by the application of a body fluid sample to the object.

The procedure can include sample preparation, sample processing and sample post-processing steps between the removal of the sample and the application of the sample to the object.

At least one object or several objects can be tracked during the procedure. Several objects can be tracked at the same time.

The object can be a good, a commodity, a semi-finished product, an object, a workpiece, a component, a foodstuff and/or a raw material.

In a human medical or veterinary test procedure, the object can be, for example, a test cassette, a test strip, a measuring strip, a reagent in a container and/or a human medical test sensor (e.g. for recording a vital sign (blood pressure, heart rate, respiratory rate, temperature) of the person or animal).

The object is changed by the procedure. This means that the object has a first state before the procedure is carried out and a second state—different from the first state—after the procedure is carried out. The procedure is responsible for changing the object from the first state to the second state.

The state of the object can be a shape, a contour, a color, an aggregate state and/or a volume.

The evaluation unit of the terminal device is set up to carry out the following, including recording, monitoring, detecting and/or accompanying, of the procedure in a computer-aided or computer-based manner, preferably exclusively computer-aided. This means that the following is carried out in particular without any other person (without human intervention apart from carrying out the procedure itself), the procedure is no longer accompanied by another person. This means that a person carrying out the procedure can be instructed by a terminal device and the correct execution of the procedure can be ensured by this method. With the method according to the invention, manipulations or errors in the course of the procedure can be reliably recognized and avoided by appropriate warnings or instructions. With the method according to the invention, a procedure cancellation and the invalidity of the procedure can be reliably detected if manipulations or errors are detected in the course of the procedure.

A person is a human being. The person, hereinafter also referred to as user, test person, procedure performer, should or would like to perform the procedure (themselves). To perform the procedure, the person requires at least part of their own body, in particular their body parts, especially their extremities. The procedure is therefore performed at least partially “manually” and therefore not fully automatically. For example, the person needs one or both arms, one or both hands, one or both legs and/or one or both feet.

The procedure can also be performed on an animal. A person who performs the procedure on the animal is a human and should or would like to perform the procedure (themselves). To carry out the procedure, the person requires at least part of the animal's body, in particular its body parts, especially the extremities. The procedure is therefore carried out at least partially “manually” and therefore not fully automatically. For example, the person needs a part of the animal's body to carry out the procedure on the animal, for example to take a sample.

The procedure is person-related, i.e. it is a direct criterion for tracking the procedure that a specific person performs the procedure.

The person is identified by the terminal device immediately before the procedure is carried out, i.e. without any significant time lag between the person carrying out the procedure (if necessary with the possible exception of object and/or aid identification and immediate procedure preparation steps).

This step to identify the person includes the recording of the person's biometric data by the terminal device. The biometric data is the result of a measurement and, if necessary, evaluation to recognize the person based on their behavioral and biological characteristics. Biometric characteristics (=biometric data) that can be used include a DNA (=mobile DNA test, genetic fingerprint) of the person, a blood count (haemogram) of the person, a fingerprint (finger line image) of the person, a gait style of the person, a facial geometry of the person, a hand geometry of the person, a hand line structure of the person, a hand vein structure of the person, an iris (iris) of the person or an iris recognition of the person. an iris recognition of the person, a body odor of the person, a body size (anthropometry) of the person, a lip movement of the person, a nail bed pattern of the person, an ear shape of the person, a retina (back of the eye) of the person, a voice of the person or speaker authentication (not equal to speech recognition) of the person or a voice recognition (tone color) of the person, a signature (static, dynamic, also handwriting) of the person, an X-ray of a set of teeth of the person and/or a dental impression of the person.

Preferably, easily recordable and technically simple (or sophisticated) verifiable biometric data of the person is used, for example facial geometry, a fingerprint and/or iris or retina recognition.

This step to identify the person also includes the comparison of the captured biometric data with biometric data from a personal data set by the terminal device. In this way, the captured biometric data is compared with stored biometric data and the identity of the person is verified.

The stored personal data set (personal profile) is preferably obtained by a method and system for setting up a personal profile, also described here, and stored on the terminal device.

As a result of this identification step, the person who wishes to carry out the procedure (immediately after identification) is identified by the procedure and also verified for the procedure using the stored personal data set.

An object to be changed by the procedure is also identified by the terminal device immediately before the procedure is carried out, i.e. without any significant time lag between the procedure being carried out by the person. As a result of this identification step, the object that is to be changed by the procedure to be performed (immediately after identification) is identified by the procedure.

In the tracking step of the method, at least one position of the person and at least one position of the object is tracked by the terminal device using methods of computer-aided gesture and/or pattern and/or image recognition and/or detection/analysis of sensor signals from the connected sensors during the performance of the procedure.

Gesture recognition is the computer-based automatic recognition of one or more gestures performed by the person using the terminal device. The methods used for this describe algorithms and mathematical procedures for recognizing the gesture. In principle, any posture of the person and any body movement of the person can represent a gesture. Gesture recognition is preferably used to recognize the person's hand and head gestures. A gesture made by the person can be recognized by the terminal device using sensors worn on the person's body or carried by the person's hand (and connected to the terminal device), in particular integrated acceleration or position sensors. Alternatively or additionally, a gesture can also be recognized using (personal) external sensors, in particular one or more detection units of the terminal device. Cameras on the terminal device (or cameras that can be connected to the terminal device) are preferably used to create images of the person. This camera-based gesture recognition uses 2D and/or 3D image analysis to recognize the person's gesture.

A distinction can be made between two types of gesture. With a continuous gesture (first type), there is a direct link between the movement observed by the terminal device and a state in the terminal device (example: pointing at the screen can control a pointer). A discrete gesture (second type) is part of a (limited) set of unique gestures, whereby each of these unique gestures is usually linked to an action.

When a gesture is recognized, the information from the sensors is fed into algorithms that analyze the raw data in order to recognize the gestures. Pattern recognition algorithms can be used here. To remove noise and reduce data, the sensor data is often pre-processed in the first step. Features are then extracted from the sensor data. These features are used as input for classification. Hidden Markov models, artificial neural networks or other artificial intelligence techniques are preferably used for this purpose.

Pattern recognition is the computer-based automatic recognition of regularities, repetitions, similarities or regularities in a data set (images, video) using the terminal device. The recognition of patterns, as the characteristics that are common to all things in a category (and therefore distinguish them from the content of other categories), can be decisive. A feature for pattern recognition can be obtained through automatic feature synthesis or empirically through learning and experience. To reduce the features obtained, it is then checked which features are relevant for class separation and which can be omitted. Finally, the features can be categorized into classes. Syntactic pattern recognition, statistical pattern recognition and/or structural pattern recognition can be used.

Image recognition—as a sub-area of pattern recognition and image processing—is the computer-based automatic segmentation of elements in an image. At least one object, person, location and/or action in a captured image can be identified. Deep machine learning can be used here, for example convolutional processors for artificial neural networks. For example, image recognition algorithms can work with the help of comparative 3D models, appearances from different angles using edge detection, which can be trained on training images with machine learning.

For example, modelling using Mediapipe based on OpenCV is used for computer-aided gesture and/or pattern and/or image recognition. This modelling is suitable for body gesture recognition and tracking (“Human Pose Detection and Tracking” or “Hand Tracking”, “Holistic Tracking”, “Face Detection”) of a person, generation of a face geometry (face mesh), object recognition and tracking (“Object Detection and Tracking” or “Hand Tracking”, “Holistic Tracking”). Explicit reference is made here to the Mediapipe publications according to the website https://google.github.io/mediapipe/.

The tracking step allows at least the position and/or the movement of the identified person and the identified object to be recognized and tracked by the terminal device with purely computer support. In this way, it is possible to track whether the person is carrying out the procedure correctly, for example whether the object is being changed correctly and/or whether the person is attempting to tamper with it or making a mistake.

In the instruction step of the method, the person is instructed to perform the procedure by the terminal device during the performance of the procedure. This instruction of the person can be carried out by displaying and audibly and/or visually outputting instructions, warnings, instructions, demonstrations, videos, images, animations, augmented reality and/or examples on a display unit (display, output unit, loudspeaker) of the terminal device. This instruction can take place at different times during the procedure, for example once at the beginning or several times in the middle of the procedure and/or at the end of the procedure. The instructions can map the sequence of the procedure in order to guide the person through the procedure according to the step sequence of the procedure.

For guidance, the terminal device can be aware of the procedure, of individual or the entirety of the sequence of the procedure, its sequence, its expected duration, its expected durations between individual or all procedure steps, and/or of an expected final result and/or one or more intermediate results.

In this way, a person is given instructions on how to carry out the procedure and the procedure is also tracked by computer—in real time.

To identify the person in the capturing step, a face of the person can be captured by a camera of the terminal device. The captured face can be analyzed by an evaluation unit of the terminal device using the or other methods of computer-aided facial pattern and/or image recognition in order to obtain the person's biometric data (to be captured). Capturing a person's face as biometric data can easily enable unambiguous determination of identity, and the methods of computer-aided face pattern and/or image recognition to be used for this purpose are reliable and require little computing power.

The identification of the person can be repeated at one or more random times during the performance of the procedure. This makes it possible to check whether the same person is still carrying out the procedure or whether—in an unauthorized manner—the person identified is no longer the person carrying out the procedure.

The recording of the person used for identification can be the recording of an image and/or one or more video (frames). This recording can be stored on the terminal device for documentation purposes. This recording can be part of a documentation of the performance of the procedure.

The tracking procedure described can be terminated with an error message if it is determined in the comparison step that the biometric data set does not match the biometric data of the personal data set (missing verification). This ensures that a personalized procedure is actually carried out by a person corresponding to a personal data set.

Immediately before the person performs the procedure, all aids to be used during the procedure can be identified by the terminal device. An aid can be any object, measuring device, component, container, sensor, tool and/or the like. The aid helps (or is necessary) to ensure that the procedure can be carried out successfully. The sensor—here as an aid—may be one of the aforementioned sensors for detecting the position or movement of the person or object. The sensor can alternatively or additionally be the test sensor described above.

Each aid used can be detected by a detection unit, for example a camera and/or the connected sensors of the terminal device. The detected aid can be analyzed by methods of computer-aided face and/or pattern and/or image recognition and/or detection/evaluation of sensor signals of the connected sensors by an evaluation unit of the terminal device, whereupon the aid can be identified.

At least one aid or several aids can be tracked during the procedure.

Identifying the object may include: Capturing an object identification data set by the terminal device; and comparing the captured object identification data set with an object data set of a background system. An object identification data set is a data set that can be uniquely assigned to an object in the process and can uniquely identify the object. This object identification data set can be, for example, a product identifier, a serial number, a barcode, a QR code, an RFID tag code, NFC tag code, etc., which can be read by the terminal device.

In one embodiment, the object identification data set is a QR code on a test cassette (=test object). The QR code can be coded and/or encrypted so that reading it does not provide any information about the systematics of the object identification data set.

It is proposed to provide the test with a machine-readable code that can be easily identified by the terminal device, e.g. QR code or tag, such as NFC tag, RFID tag, Bluetooth tag, etc.

The machine-readable code can, for example, be applied during production of the test and then entered into the background system 40 by machine reading.

Alternatively, according to the invention, it is also envisaged that a test without machine-readable code (so-called system-neutral test) is acquired. In addition, a machine-readable code is acquired in order to be able to participate in the method/system according to the invention. During the preparation of the test, the machine-readable code is applied to the test (the test cassette). For example, a QR code is placed as a sticker on the test cassette as a machine-readable code by an adhesive layer.

During the procedure, the presence of the machine-readable code is queried at regular or random intervals or at fixed times during the procedure. If the machine-readable code cannot be read by the terminal device during these steps, the person is prompted to correct the position of the test. If a correction is not made in time, the test is cancelled and the test is discarded as “invalid” and the serial number of the test is marked as “used/used” in the background system.

To identify the object in the capture step, a machine-readable data set can be captured by a capture unit, in particular a camera of the terminal device. A machine-readable data set can be a serial number applied to the object, which is captured by the capture unit. The serial number can be a production number from the manufacture of the object. The serial number may be a serial number independent of the object from a third party that provides the method and system for computer-aided tracking. The captured serial number can be analyzed by the evaluation unit, for example optical character recognition (OCR) can be used. The machine-readable data set can be a character code applied to the object, which is captured by the capture unit. The character code, for example a barcode or a QR code, can be analyzed by the evaluation unit. The machine-readable data set can be a data set encoded in an RFID tag or an NFC tag, which is captured by the capture unit, in particular an NFC reading unit or RFID reading unit. The character code, for example a barcode or a QR code, can be analyzed by the evaluation unit.

The machine-readable data set of the object can be validated by querying a corresponding object data set in a background system. The machine-readable data set of the object is thus registered in a background system. The machine-readable data set of the object is thus registered in a background system. In this way, the authenticity of an object, a type of object, a production date, a manufacturer ID, a validity date, an object property and/or a certificate can be queried and/or verified. To validate the object, the machine-readable data set is compared with the object data set and if there is a match (and/or if it merely exists in the background system), the object is validated as genuine and is released for the procedure.

After validation of the machine-readable data set, the object data set or a procedure identification data set can be invalidated in the database of the background system by changing a status. This ensures that the object is unique.

In the tracking step, the at least one position and/or movement of the person, preferably also a movement of a body part (limb) of the person and the at least one position and/or movement of the object can be detected by a camera as the detection unit of the terminal device. The recorded position and movement of the person and the object are analyzed by an evaluation unit of the terminal device using methods of computer-aided gesture and/or pattern and/or image recognition. This makes it possible to check whether the person performs a movement corresponding to the procedure and/or whether the object is changed in accordance with the procedure. Incorrect or false movements of the person or the object can thus be checked.

A movement radius of the person and object can also be tracked. If it is recognized that the person and/or object is outside a predefined position frame, the procedure can be aborted and the procedure considered invalid, for example when leaving an area that can be captured by a camera or a previously defined area. The person is thus forced to remain within a predefined movement radius and a predefined movement pattern. It is also possible to check whether the movement sequences of the object match the procedure to be carried out. Incorrect positions or movements are recognized in this way.

The previously defined area is determined and specified before the procedure is carried out, for example. The area that can be captured by the camera is determined and defined before the procedure is carried out, for example. The areas can be indicated on a display of the terminal device by displayable auxiliary lines.

In the tracking step, an object property of the object can be captured using a camera of the terminal device. The captured object property of the object can be analyzed by an evaluation unit of the terminal device using methods of computer-aided face and/or pattern and/or image recognition.

The analyzed object property of the object can be compared with an object property to be expected during the procedure, for example in real time. For this purpose, changes in the state of the object during the procedure, i.e. changes in shape, color and/or conditions or intermediate and/or final results, should be known to the terminal device.

The procedure can be terminated with an error message if it is determined in the analyzing step that the detected position of the person and/or the detected movement of the person and/or the detected position of the object and/or the detected movement of the object are outside the fixed specifications of the procedure. For example, if the person or a body part (one hand, both hands) leaves a detection area of the terminal device's camera, the procedure could be considered invalid. For example, if the object leaves a detection range of the terminal device's camera, the procedure could be considered invalid.

To document the execution of the procedure, the execution of the procedure can be recorded and saved at one or more random times during the execution of the procedure using a camera on the terminal device. This creates procedure documentation in real time, which can be stored in a procedure identification data set in the terminal device and can be retrieved at any time later. Validation of the procedure can also be (re-)checked based on this procedure documentation, for example.

The procedure documentation comprises, for example, one or more random recordings of the person and/or the object during the execution of the procedure and/or a procedure step. Individual images (a video frame) or several video frames (as one or more videos) can be taken by the camera of the terminal device of the person during the process step. The procedure documentation comprises, for example, images of the person and/or the object during the execution of the procedure and/or a procedure step that are deliberately instructed by the evaluation unit.

A procedure result for an antigen test of a virus detection test (as a procedure) may comprise the display of a bar on a test strip of a test cassette (as an object). This bar can be detected and analyzed by the detection unit of the terminal device. The presence or absence of a bar at a predefined position on the test cassette indicates the procedure result. For example, the presence of the bar can mean that the procedure result is “positive”. The absence of the bar can mean that the procedure result is “negative”.

In addition, a control bar can be provided in the test cassette, which serves as a self-test of the test strip. The presence of the control bar can mean that the test cassette is working properly. The absence of the control bar may mean that the test cassette is faulty.

In one embodiment, several bars can be present on a test cassette (object) to display several procedure results. In this case, a number of tests are carried out in parallel or at different times with one body fluid (of the person or animal). Each procedure result is displayed with its own bar on the test cassette. The presence or absence of a bar at a predefined position on the test cassette for this procedure result indicates the procedure result. In this way, various tests can be carried out by taking/collecting just one sample from a body fluid.

In one embodiment, several bars can be present on a test cassette (object) to represent a procedure result. The number of bars can be used to represent a concentration of infectivity of the body fluid, for example as a percentage, e.g. one bar for low, two bars for medium, three bars for high (concentration in relation to a reference concentration of infectivity).

For the guidance, guidance instructions can be displayed on a display of the terminal device, whereby a guidance instruction can be provided depending on the execution of the procedure and/or depending on the tracking of positions and movements of the person and the object by the terminal device during the execution of the procedure.

According to the invention, there is also provided a method for computer-aided validation of a procedure carried out by a person and tracked by a computer. The method comprises the following steps: requesting a validation of a procedure result by a person input at a terminal device; generating and sending a validation request to a background system comprising a scatter value over the person data set and a procedure identification data set of the procedure result to be validated; receiving in the terminal device a validation token from the background system; displaying a machine-readable code based on the validation token and displaying the procedure result on a display unit of the terminal device; checking the machine-readable code by a checking entity while checking the validation token; displaying a validation result relating to the procedure result together with the procedure result and the scatter value over the personal data set and preferably procedure information on a display unit of the checking entity.

This validation procedure is applied to a procedure carried out by the person. The execution of the procedure has generated a procedure result. The procedure result can be the changed object (the second state of the object after the procedure has been carried out).

The person selects the procedure result to be validated by entering it on the terminal device. The terminal device then generates a validation request. The person who selects the validation request and whose procedure result is to be shown is preferably identified again by the terminal device. This is particularly necessary if the validation instance is not a person but a machine (barcode scanner).

This validation request includes a scatter value of the personal data set that is stored in the storage unit of the terminal device. The personal data set is uniquely assigned to the person who carried out the procedure. The scatter value (=hash) of the validation request can be obtained using a cryptographic scatter value function that can be applied to the personal data set by the evaluation unit of the terminal device. A hash function or scatter function is a mapping that maps the personal data set to the scatter values (=hash values). A hash function is not injective.

This validation request also includes a procedure identification data set, or procedure ID for short. This procedure ID is uniquely assigned in the procedure and system to a procedure that is yet to be carried out and/or has already been carried out. This makes the procedure more forgery-proof and tamper-proof.

The background system receives the request and generates a validation token. The validation token is sent back to the terminal device.

A machine-readable code generated by the terminal device is now displayed on a display unit of the terminal device. The validation confirmation was created using the validation token. The procedure result is also displayed. The machine-readable code (as a type of unconfirmed validation confirmation) with the validation token and a link to the validation server can be displayed together with the procedure result on the display unit of the terminal device.

To validate the procedure result, a verifier, for example a capture unit (camera, QR scanner, barcode scanner) of a terminal device or vending machine, reads the machine-readable code (the unconfirmed validation confirmation) and checks the validation token for validity. A verifier, for example an auditor or a vending machine with a scanning function, reads the machine-readable code. When it reads the code, the verifier issues a validation request and checks the validation token. If the validation token is valid, the background system can retrieve the procedure result, a scatter value via the personal data set and, preferably, procedure information.

A positive validation result is displayed on a display unit of the checking instance together with the procedure result and data from the background system if the validation token could be decoded, time-validated and checked (in the sense of validated, i.e. the procedure was carried out by this person)

A negative validation result is displayed on a display unit of the validation instance together with the procedure result and data from the background system if the validation token could not be decoded or if the token is invalid (timeout) or if the validation is not successful.

The computer-aided traced procedure is preferably one that has been traced using the method (or system) for computer-aided tracing of a procedure to be performed by a person according to the preceding type. The validation method is thus connected to the tracing method, which means that both tracing of the procedure and validation of the procedure can be carried out in a computer-aided, tamper-proof manner and without another person.

The checking entity can be a person or a system (terminal, machine, barcode scanner with evaluation unit). The system can be an admission control system for a building (restaurant, concert hall, theatre, etc.) or a facility (stadium, concert area, restaurant, etc.). The system can check whether the validation token has been successfully validated and can pass the identified person if the procedure has been validated.

In a preferred embodiment, the validation request comprises a procedure documentation record that was created when the procedure was performed.

The procedure documentation data set includes, for example, random recordings of the person and/or object during the procedure. The procedure documentation data set includes, for example, deliberately instructed recordings of the person and/or object during the procedure. Individual images (a video frame) or several video frames (as one or more videos) can be taken by the camera of the terminal device of the person during the process step.

Preferably, the validation token can define a validity period for the validation result with regard to the procedure result. For example, the validity period can be 1 minute, 2 minutes, 5 minutes, 10 minutes, 15 minutes or 20 minutes. The validity period is encoded in the validation token. If the validity period has expired, the validation result is negative. The person must then carry out a new validation.

The number of validations according to the above procedure is not limited. This means that a procedure result can be validated as often as required. A validation that has already been carried out is logged in the background system and cannot be called up a second time.

The validation result may include procedure information. This procedure information is information from the background system that the validator received from the background system when checking the machine-readable code. This provides the validator with additional information about the procedure execution. The procedure information can be a procedure date and/or a procedure time according to the background system. For example, if the procedure is a human medical test procedure, then it is possible to specify when the procedure was carried out. For example, in the case of a virus detection test, it is possible to determine whether the procedure result is still meaningful. For example, the procedure information provides information (at least indirectly) that the test procedure is invalid and a new test procedure must be performed.

The procedure identification data set is preferably generated by the terminal device and communicated to the background system. Alternatively, the procedure identification data set is generated by the background system and communicated to the terminal device at the start of the procedure.

The machine-readable code is preferably a QR code. The verification instance comprises a detection unit to read the machine-readable code.

The procedure result can be queried by the background system.

The invention further comprises a computer-aided validation of a procedure performed by a person. The system comprises an input unit of a terminal device adapted to receive a person's input for requesting validation of a procedure result. The system further comprises a control unit adapted to generate a scatter value over a person data set stored in a storage unit of the terminal device; generate a validation request comprising the scatter value and a procedure identification data set of the procedure result to be validated; send the validation request to a background system; and receive in the terminal device a validation token of the background system. The system further comprises a display unit of the terminal device, adapted to display a machine-readable code with the validation token and the procedure result. The system further comprises a verification entity adapted to verify the machine-readable code by checking the validation token and displaying a validation result relating to the procedure result together with the procedure result and the scatter value over the personal data set and preferably procedure information.

According to the invention, there is also provided a method for setting up a person profile for use in a method of the preceding type. The method comprises capturing data of an identification document of a person by a terminal device; evaluating the data of the captured identification document by an evaluation unit of the terminal device to obtain a personal data set comprising biometric data and personal data of the person; and storing the obtained personal data set as a personal profile in a storage unit of the terminal device.

The personal data are in particular a first name, a surname, a date of birth, a place of birth, an address (address), an academic degree and/or a document number.

An identification document is, for example, an identity card, a passport, a driving license and/or a company ID card. The identification document includes, in particular, a photograph of the person.

In the capture step, a photograph of the identification document can be captured by a camera of the terminal device. In the evaluation step, the captured light image can be analyzed by the evaluation unit of the terminal device using methods of computer-aided face and/or pattern and/or image recognition in order to obtain the biometric data of the personal data set.

In the capture step, personal data from the identification document can be captured using a camera on the terminal device. In the evaluation step, the captured personal data can be analyzed by optical character recognition by the evaluation unit of the terminal device in order to obtain the personal data of the personal data set.

In the capture step, an electronic data storage of the identification document can be read out. In the evaluation step, the read data can be analyzed to obtain the personal data set. Optionally, the data can only be read out after prior PIN verification by the person entering a PIN.

In the storage step, the personal data set obtained can be stored cryptographically secured in a storage unit of the terminal device.

According to the invention, a system for setting up a personal profile comprising a terminal device and an identification document is also provided. The system comprises: an acquisition unit of the terminal device, set up for acquiring data of an identification document of a person; an evaluation unit of the terminal device, set up for evaluating the data of the acquired identification document to obtain a personal data set comprising biometric data and personal data of the person; and a storage unit of the terminal device, set up for storing the obtained personal data set as a personal profile.

The capture unit of the terminal device can be a camera, wherein the camera is set up to capture a light image of the identification document. The evaluation unit of the terminal device may comprise a face recognition module, wherein the face recognition module is set up to analyze the captured light image using methods of computer-aided face and/or pattern and/or image recognition in order to obtain the biometric data of the personal data set.

The capturing unit of the terminal device may be a camera, wherein the camera is set up to capture personal data of the identification document. The evaluation unit of the terminal device may comprise a character recognition module, wherein the character recognition module is set up to analyze the captured personal data by computer-aided face and/or pattern and/or image recognition methods in order to obtain the personal data of the personal data set.

The identification document can comprise an electronic data storage, wherein the acquisition unit of the terminal device is a reading unit, wherein the reading unit is set up to read out the electronic data storage of the identification document; and wherein the evaluation unit of the terminal device is set up to evaluate the read-out data in order to obtain the personal data set.

The evaluation unit can comprise an encryption module, whereby the encryption module is set up to cryptographically secure the personal data set received. The storage unit of the terminal device can be set up to store the cryptographically secured personal data set.

Communication between the terminal device and the background system takes place via TCP/IP protocol. The background system comprises a server architecture on which a database runs. The database manages an object data set that is uniquely assigned to an object, a procedure ID for each procedure and a person data set for each person.

During process execution, the user is guided through the test procedure, whereby the user is monitored during the process to ensure that all steps are carried out correctly and also to document the execution of the process. In addition, the process flow is automatically controlled by the user's activities. In order to orchestrate these four functionalities (control, monitoring, documentation, management) in a test procedure application on a mobile device, a test procedure application architecture was developed that allows individual process steps to be managed and interpreted on the basis of available technology modules that are assembled on a video stream (similar to Lego bricks).

Individual technology modules allow partial functionality, but only the orchestration of all modules together guarantees the continuous process flow using the video stream to control, monitor, document and manage the application flow.

Some of these modules can themselves be assembled from sub-modules. The underlying video stream can switch between the front and rear camera of the terminal device and between fixed observation positions, with the terminal device positioned on a stand, for example, to (1) ensure a calibrated position with consistent angles and (2) enable user-based tracking of specific components/tools used in the process.

The architecture enables a new type of application for mobile device 30, where various video and AI functionalities are combined to manage more complex tasks or processes, such as controlling, monitoring, documenting and/or managing a test procedure through the test procedure application of mobile device 30.

Thanks to the developed application architecture, all steps can now run in parallel to the process execution and the disclosure of personal data is avoided.

### SHORT DESCRIPTION OF THE FIGURES

In the following, the invention and further embodiments and advantages of the invention are explained in more detail with the aid of figures, whereby the figures merely describe embodiments of the invention. Identical components in the figures are labelled with identical reference signs. The figures are not to be regarded as true to scale; individual elements of the figures may be shown in exaggerated size or exaggeratedly simplified. Elements shown as dashed lines are optional.

FIG. 1 shows a flow chart of a method 100;

FIG. 2 shows a system 1000;

FIG. 3 shows a flow diagram of a method 200;

FIG. 4 shows a system 2000;

FIG. 5 shows a method 300;

FIG. 6 shows a system 3000;

FIG. 7a-f each show a display element of a terminal device;

FIG. 8a-k shows a flow chart of the methods 100, 200, 300;

FIG. 9a-f shows exemplary structures for different data sets of the methods 100, 200, 300;

FIG. 10a-b shows two alternative embodiments of process step 2044a;

FIG. 11a-j shows a further flow chart of the methods 100, 200, 300;

FIG. 12 modular high-level architecture of the test procedure application;

FIG. 13a to 13c show different versions of the test procedure application architecture of FIG. 12; and

FIG. 14 shows a validation procedure for test certificates.

### DETAILED DESCRIPTION OF DESIGN EXAMPLES

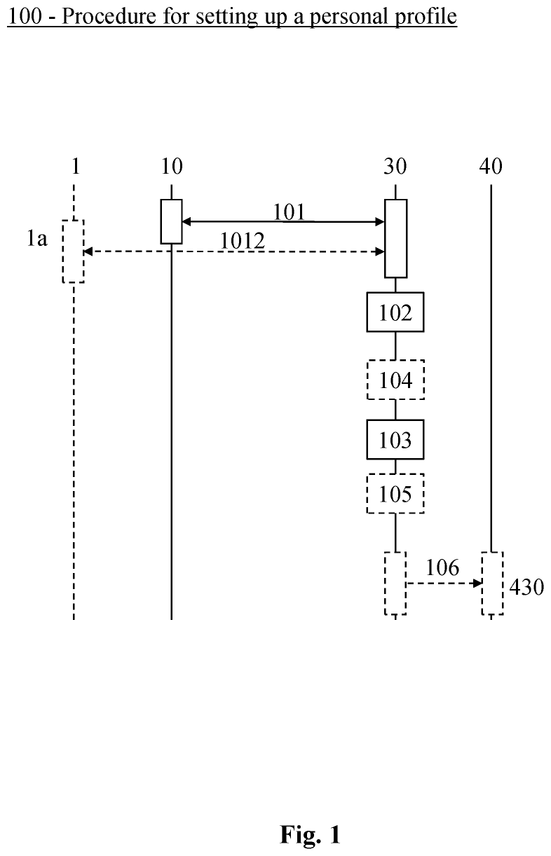

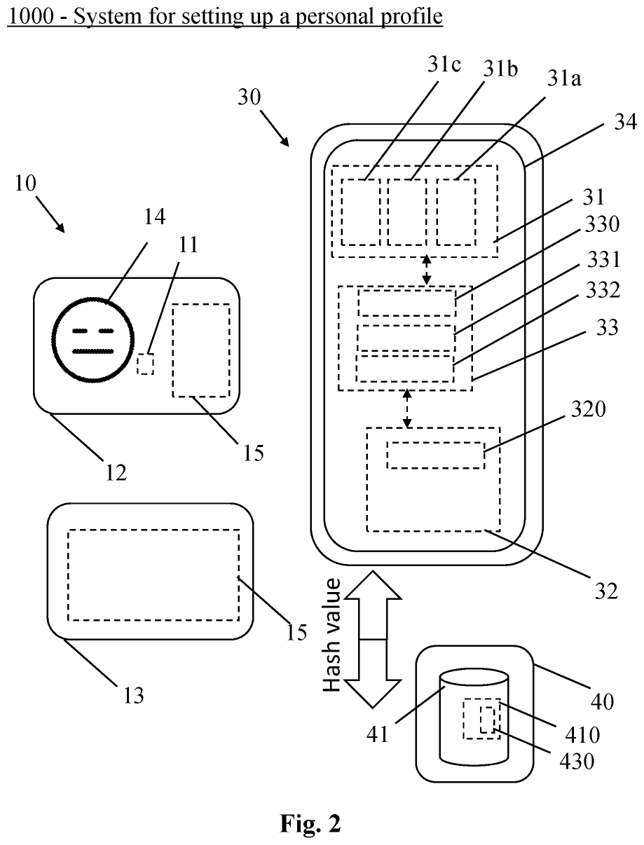

FIG. 1 shows a flow chart of a method 100 for setting up a personal data set (=personal profile). Elements shown in dashed lines are optional. FIG. 2 shows a system 1000 for setting up a personal profile comprising a terminal device 30 and an identification document 10. The description of FIG. 1 also refers to the system 1000 of FIG. 2.

In a first step 101, the method 100 of FIG. 1 comprises capturing data 11, 14, 15 of an identification document 10 of the person 1 by a terminal device 30. The terminal device 30 has a capturing unit 31 for this purpose.

The identification document 10 is, for example, an identity card, a passport, a driving license and/or a company ID card of the person 1 whose personal data set 320 is to be set up in the terminal device 30.

The identification document 10 can have a photograph 14 of the person 1. The photograph 14 may be arranged on a front side 12 of the identification document 10.

The identification document 10 can have personal data 15 of the person 1. The personal data 15 may be arranged on a front side 12 and/or on a back side 13 of the identification document 10. Personal data 15 of a person 1 may comprise one or more of the following: first name of person 1, last name of person 1, birth name of person 1, birthday of person 1, place of birth of person 1, address of person 1, nationality of person 1, handwritten signature of person 1, eye color of person 1, height of person 1, identification document number of identification document 10, identification document type of identification document 10, date of issue of identification document 10, issuing authority of identification document 10, validity date of identification document 10.

The identification document 10 can have a data storage 11 with biometric data of the person 1 and personal data 15 of the person 1. The biometric data are characteristics of the person 1 that are so unique that this person 1 can be identified on the basis of these characteristics. The biometric data on a German ID card as an example of an identification document 10 of a person 1 are the facial geometry of the face 1a of the person 1, the fingerprints of the person 1 and/or the handwritten signature of the person 1.

Further possible biometric data of a person 1 (also with regard to the methods 200 and 300, which are described below) can be: a DNA (=mobile DNA test, genetic fingerprint) of the person 1, a blood count (hemogram) of the person 1, a fingerprint (finger line image) of the person 1, a gait style of the person 1, a hand geometry of the person 1, a hand line structure of the person 1, a hand vein structure of the person 1, an iris (iris) of the person 1 or an iris recognition of person 1, a body odor of person 1, a body size (anthropometry) of person 1, a lip movement of person 1, a nail bed pattern of person 1, an ear shape of person 1, a retina (back of the eye) of person 1, a voice of person 1, respectively Speaker authentication (not the same as speech recognition) of person 1 or voice recognition (tone color) of person 1, a signature (static, dynamic, also handwriting) of person 1, an X-ray of a set of teeth of person 1 and/or a dental impression of person 1.

The detection unit can be a camera 31a. The camera 31a can be a front camera of the terminal device 30, which is arranged on the side of a display unit 34 (display) of the terminal device 30. The camera 31a can be a rear camera of the terminal device 30, which is arranged on a side opposite the side of the display unit 34 (display) of the terminal device 30.

The detection unit 31 can be a reading unit 31b. The reading unit 31b is set up to receive electronic data from the data storage 11 of the identification document 30 (or also of an object 20 according to the methods 200, 300). Receiving takes place, for example, using near-field communication means (NFC, RFID) as reading unit 31b. A PIN request may be necessary to read the data from the data storage 11 of the identification document 10.

The capture unit 31 can be a code scanner 31c. The code scanner 31c is set up to capture machine-readable data sets, such as barcodes, QR codes, etc., from the identification document 10 (or also from an object 20 according to the methods 200, 300).

In a subsequent step of FIG. 1, the data 11, 14, 15 of the captured identification document 10 is evaluated 102 by an evaluation unit 33 of the terminal device 30 to obtain a personal data set 320 comprising biometric data and personal data of the person 1. The evaluation unit 33 is, for example, a CPU of the terminal device 30. The evaluation unit is set up to execute methods of computer-aided face, pattern, character and/or image recognition. During execution, the captured data 11, 14, 15 of the identification document 10 are analyzed.

Thus, the captured photograph 14 of the identification document 10 can be analyzed by a face recognition module 330. For this purpose, the face recognition module 330 analyses the captured photograph 14 and extracts facial feature data of the person 1 as biometric data of the personal data set 320.

Thus, the captured personal data 15 of the identification document 10 can be analyzed by optical character recognition (OCR) of a character recognition module 331. The character recognition module 331 analyses the captured personal data 15 and extracts it as personal data of the personal data set 320.

Thus, the captured data can be obtained from the data storage 11 of the identification document 10 by NFC communication in the terminal device 30 and this captured data comprises both the biometric data and personal data of the personal data set 320.

In an optional step 104, the obtained personal data set 320 is cryptographically encrypted using the encryption module 332 of the evaluation unit 33 of the terminal device 30.

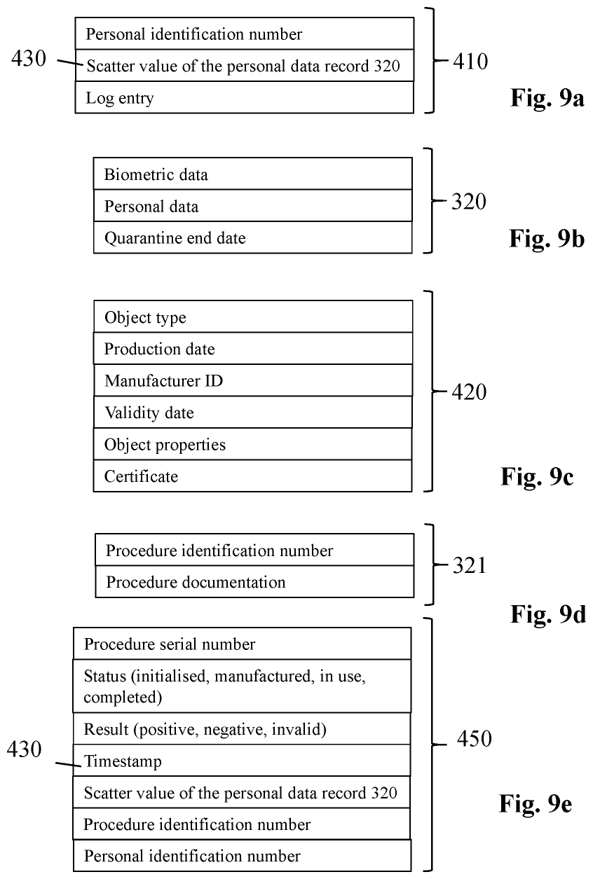

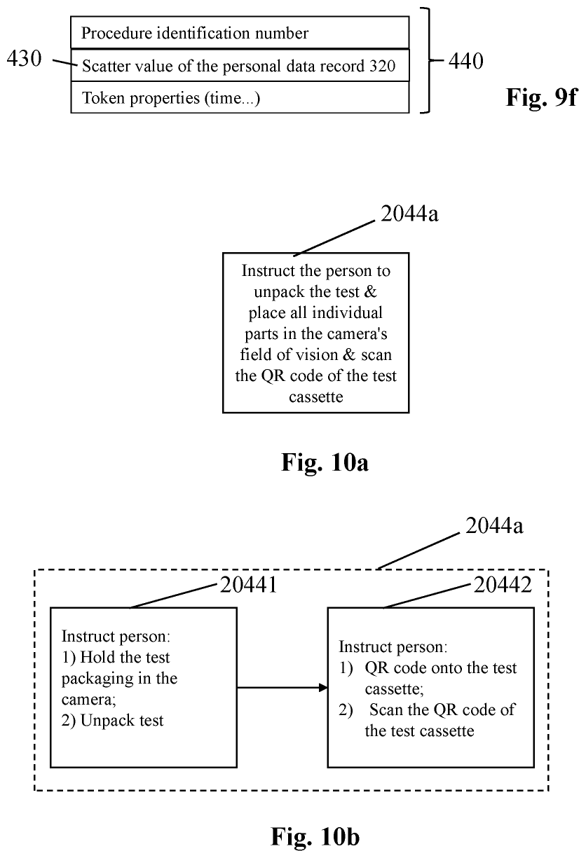

In a storing step 103, the obtained personal data set 320 is stored as a personal profile in a storage unit 32 of the terminal device 30, possibly cryptographically secured if step 104 has also been executed. A structure of an exemplary personal data set 320 is shown in FIG. 9b.

In the optional step 105, a scatter value (=hash value) of the personal data set 320 is generated by of the evaluation unit 33, for example also with the module 332 of the evaluation unit 33 of the terminal device 30.

In the optional step 106, the scatter value of the personal data set 320 generated in step 105 is sent from the terminal device 3 to a background system 40. In the background system 40, this scatter value 430 can be stored in a database 41. The scatter value 430 can be part of an anonymized personal data set 410 in the background system 40. An exemplary structure of an anonymized personal data set 410 is shown in FIG. 9a.

In addition to the scatter value 430, the anonymized personal data set 410 (=“Remote_Testee”) can also include a log entry with which procedure events, in particular failed object identifications, can be stored with a time stamp.

The anonymized personal data set 410 (=“Remote_Testee”) can also include a personal identification number with which the person 1 can be uniquely referenced on the background system 40.

Alternatively, the entire personal data set 320 (biometric data and personal data) is stored in the background system 40 in step 106. For this purpose, the background system 40 is preferably a trusted server that has appropriate data integrity and tamper resistance for managing such personal data sets 320.

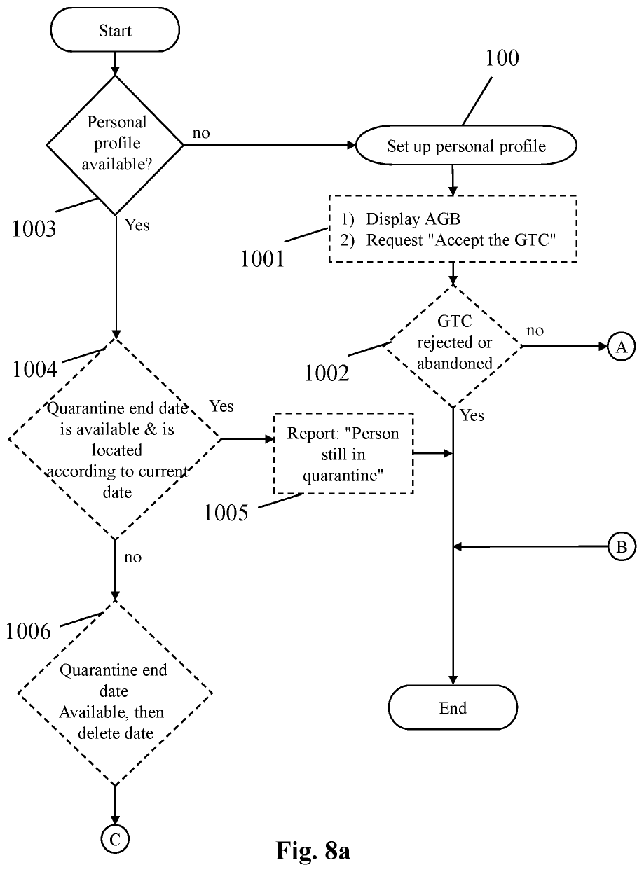

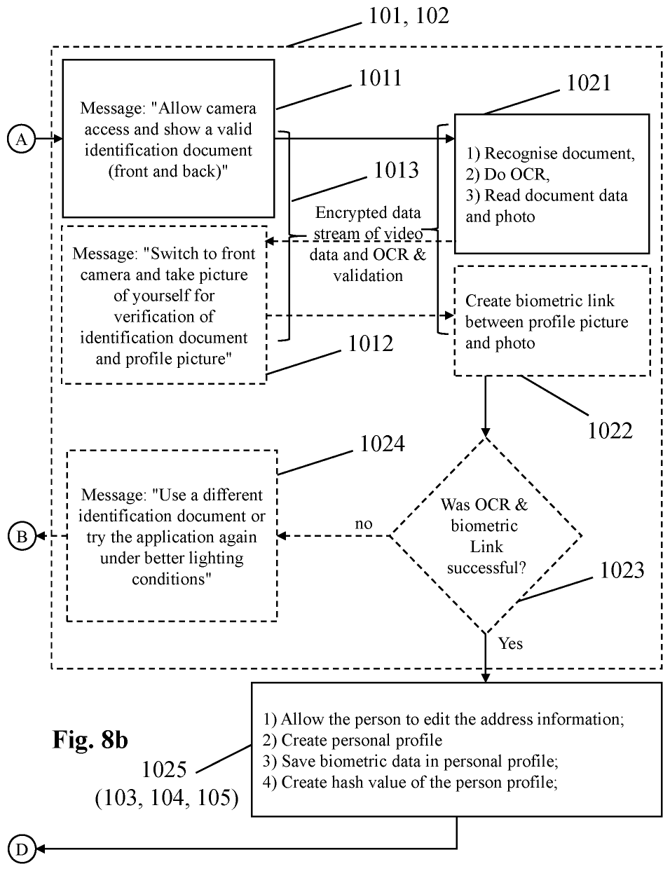

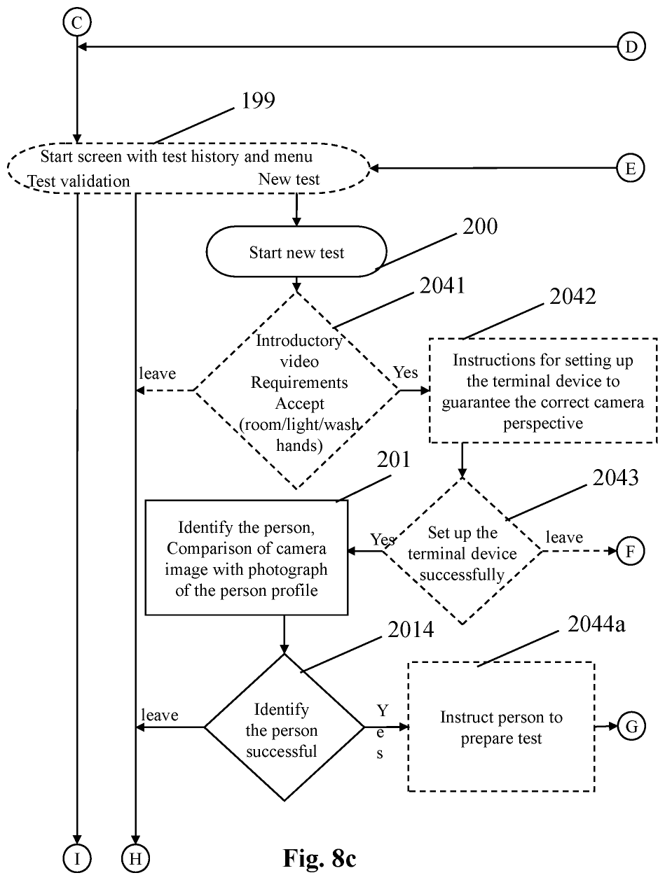

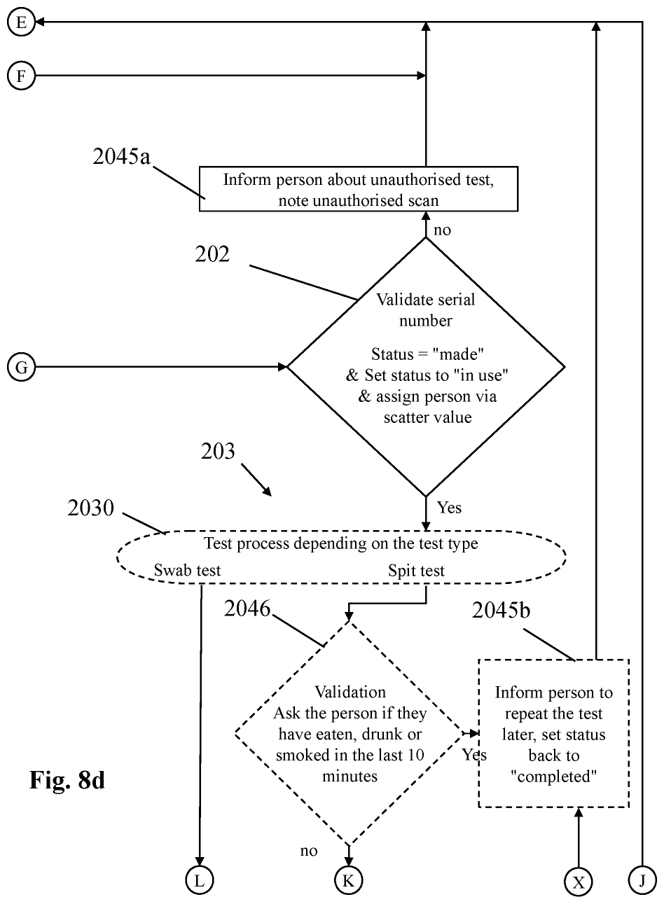

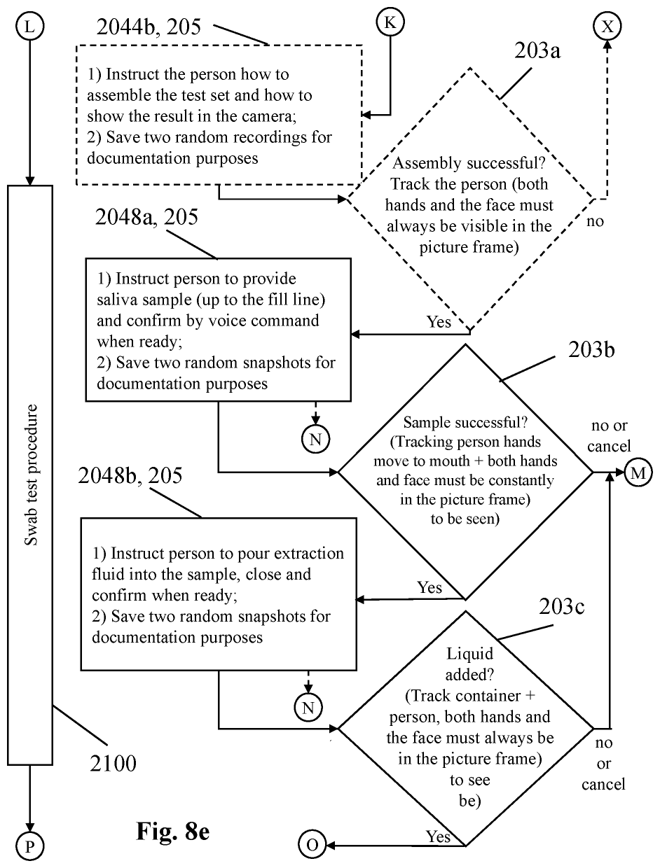

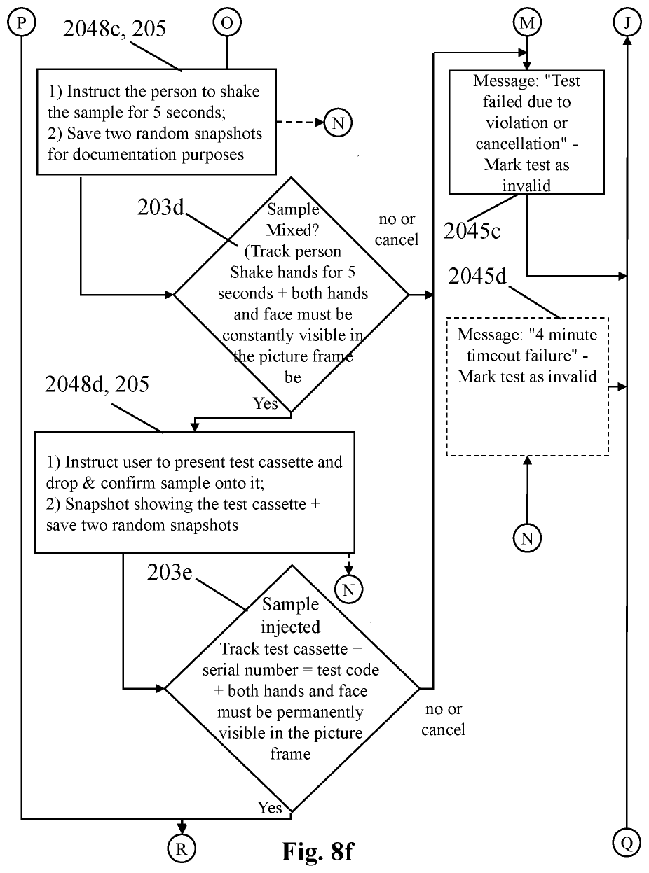

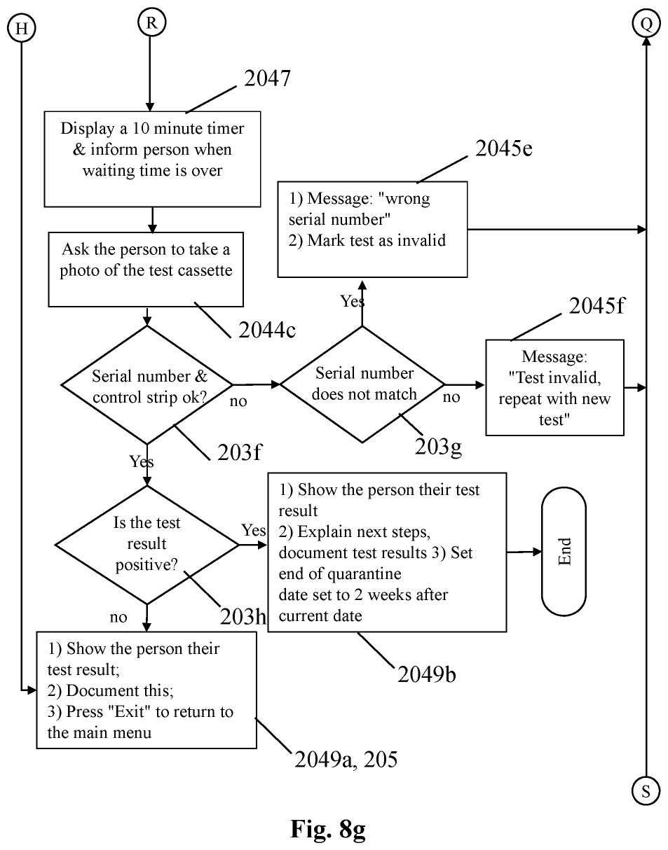

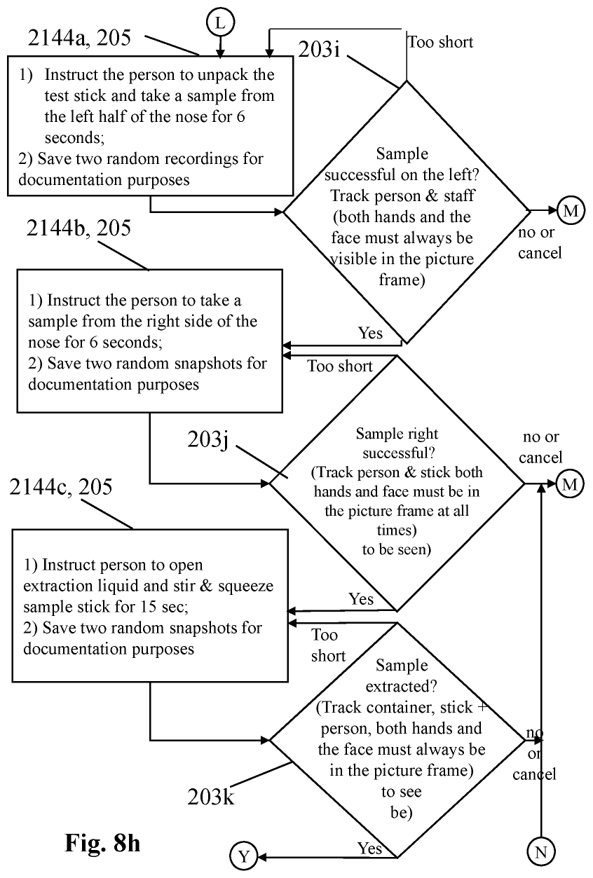

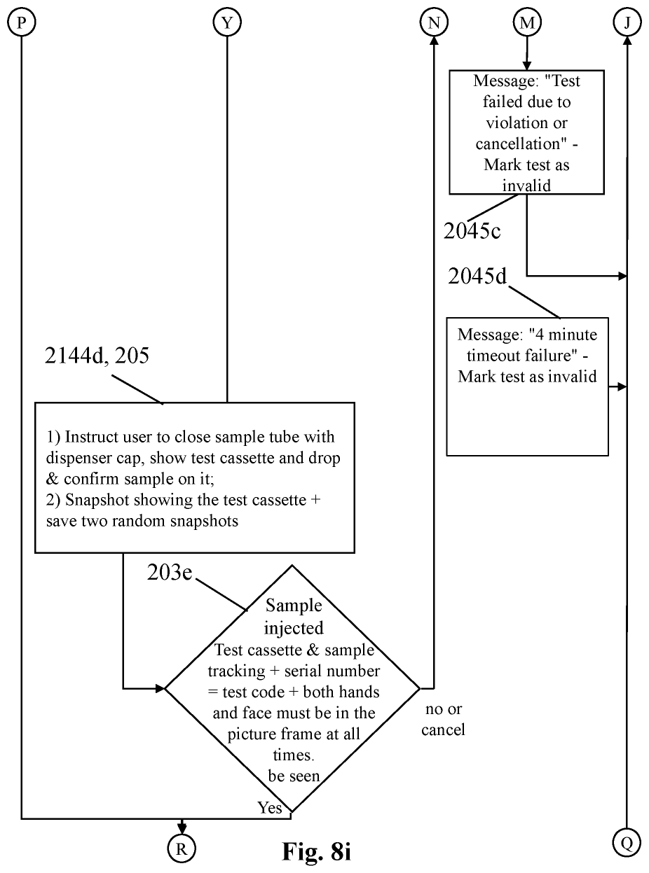

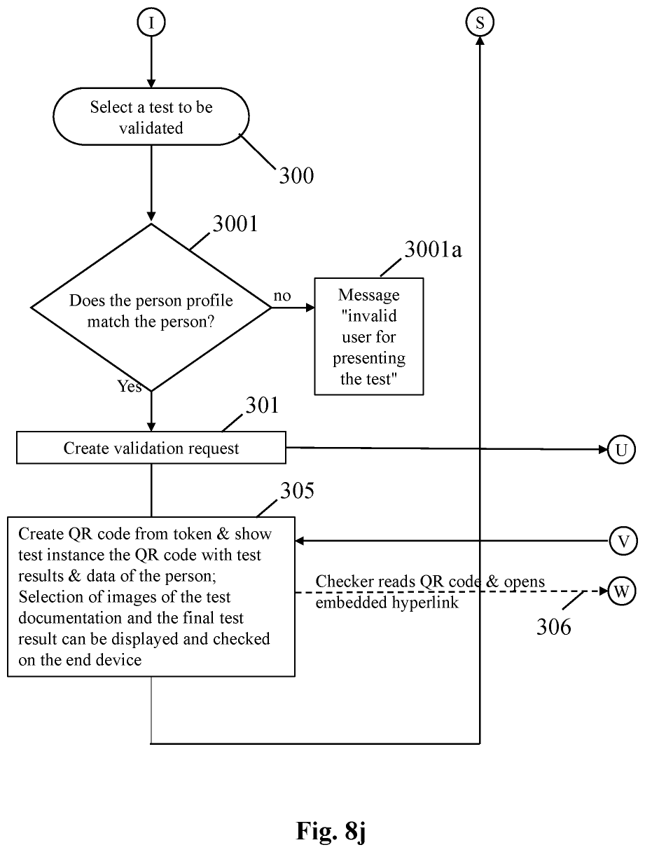

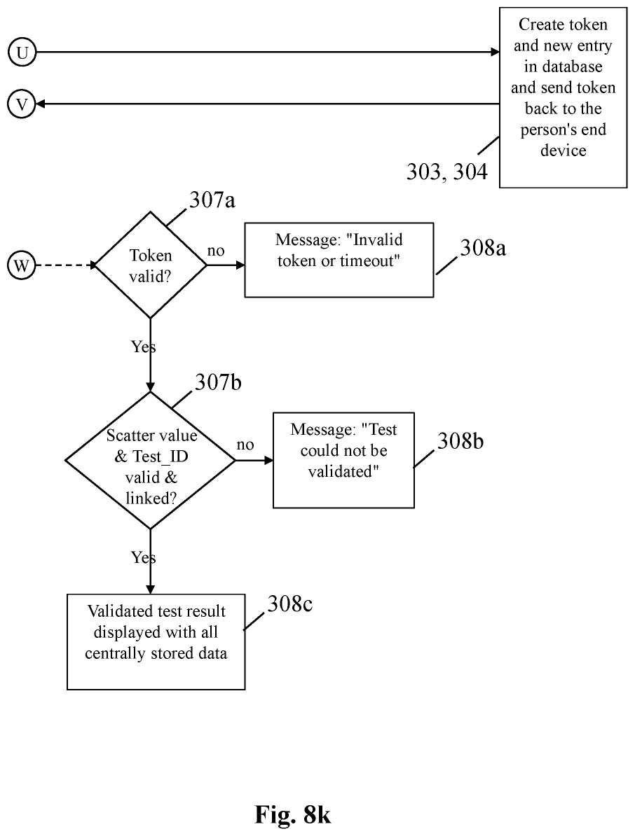

An optional step 1012, which is explained in more detail in the flowchart of FIG. 8, is shown as a dashed line in FIG. 1. In the optional step 1012, after capturing the data 11, 14, 15 of the identification document 10, the terminal device 30 can request the person 1 to capture an image of the face 1a of the person 1 from the capturing unit 31 of the terminal device 30. This captured face 1a of the person 1 is compared by the terminal device 30 in steps 1022, 1023 of FIG. 8b with the biometric data obtained from the identification document 10 in order to verify that the person 1 on the identification document 10 is also the person 1 in front of the terminal device 30. This additional verification step (steps 1012, 1022, 1023 of FIG. 8e) increases the security of the method 100, 200 and 300 and serves to securely establish the identity of the person 1.

Preferably, the biometric data of the person 1 is linked to the personal data set 320 instead of the biometric data of the photograph in order to map lighting conditions and a changed optical appearance of the person 1 compared to the photograph 1 in the process and to facilitate the recognition of the person 1.

The method 100 for setting up a personal data set 320 for a test procedure to be carried out for an antigen test for SARS-CoV-2 is shown in FIGS. 8a and 8b and is described later.

FIG. 2 shows the system 1000 for setting up a personal profile 320 comprising the terminal 30 and the identification document 10. Reference is made to the explanations relating to FIG. 1 in order to avoid unnecessary repetition.

The terminal device 30 can be a smartphone of the person 1. This terminal device 30 is carried by the person 1 at all times and can be used flexibly for procedure tracking (method 200) and procedure validation (method 300). Smartphones now have sufficient processor and computing power in their evaluation units 33, e.g. a CPU, a GPU or additional signal processors, that computer-aided face and/or pattern and/or image recognition based on methods of artificial intelligence and machine learning can be reliably carried out.

The terminal device 30 of the systems 1000, 2000 and 3000 should be a terminal device 30 that supports an operating system from iOS™ version 12 and/or from Android™ version 9 (Pie) or later.

To make the monitored and certified procedure tamper-proof, the entire data set for a user is divided into (1) data stored in the test procedure application, in particular personal data, and (2) centrally stored data, in particular non-personal data.

According to the invention, the memory architecture of FIGS. 2, 4 and 6 is based on three components:
(1) Personal data stored in a storage unit, for example as personal data set 320, such as name, date of birth, address, document number, document type, profile picture, and also documentation images of test procedures performed together with a biometric profile.
(2) A scatter value (e.g. hash value) 430 over the entire personal profile data set is used as a cryptographic key between the personal data from (1) on the terminal device 30 and the centrally stored non-personal data. This hash value 430 makes it possible that a person 1 cannot be traced, since the hash value 430 is only based on data fragments of the personal data. Even if the same person 1 were to recreate the personal data set 320, the hash value 430 would change when a new profile picture is taken that is different from the first profile picture.

In addition, the hash value key 430 is implemented unidirectionally, so that the test procedure application of the terminal device 30 can retrieve and write centrally stored non-personal data. In contrast, there is no application programming interface, API, via which the test procedure application can access personal data via the hash key.
(2) Non-personal data is stored centrally in a storage 41 of the background system 40 and linked to the personal data, such as the data 320 or the images, via the hash value key 430. Non-personal data are, for example, anonymous users, the number of failed test procedures and all test-related data from tests carried out, such as the type of test manufacturer, test time, test date, test location (at postcode level), test result. The hash value key allows all decentrally stored data to be assigned to an anonymous user—which makes it possible to generate real-time statistics on the overall process.

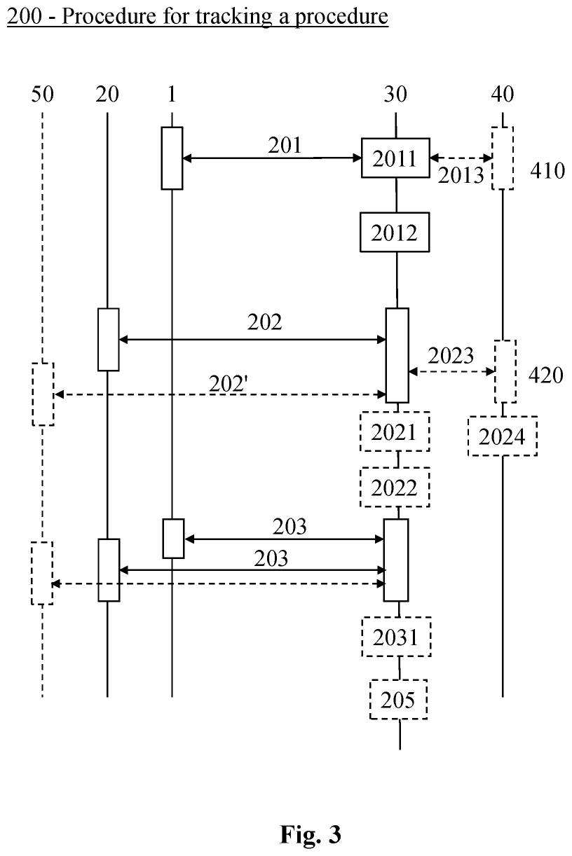

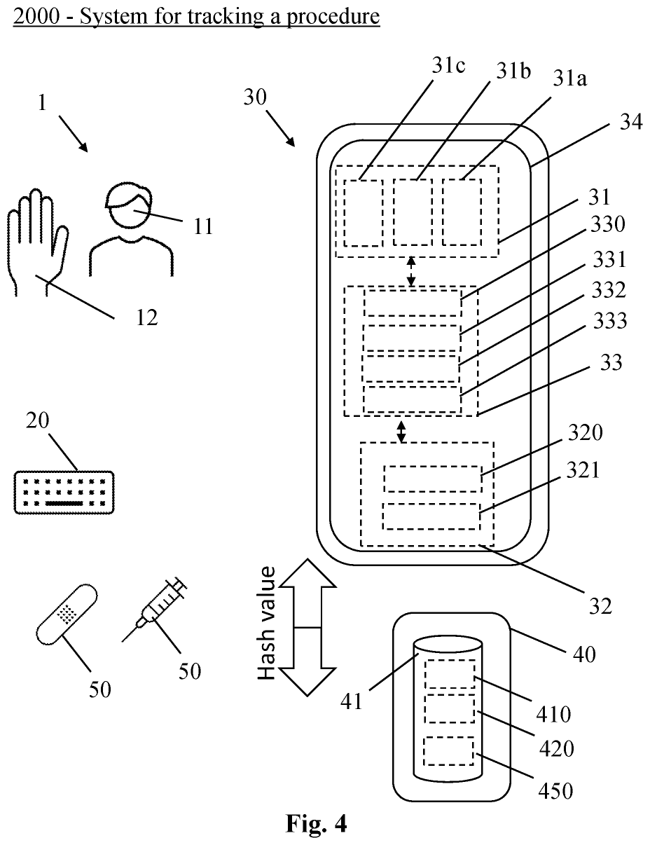

FIG. 3 shows a flowchart of a method 200 for computer-aided tracking of a procedure to be performed by a person 1. Elements shown in dashed lines are optional. FIG. 4 shows a system 2000 for computer-aided tracking of a procedure to be carried out by person 1. The description of FIG. 3 also refers to the system 2000 of FIG. 3. The procedure 200 can be directly connected to the procedure 100 of FIG. 1. An exemplary connection is shown in FIG. 8c.

In a step 201, the person 1 is identified by an identification unit of the terminal device 30 immediately before the person 1 performs the procedure. For this purpose, the biometric data of the person 1 is captured by the terminal device 30 in a capture step 2011. This capture in step 2011 can be carried out on the basis of the same technologies as the capture of the biometric data from the identification document 10, with the only difference that now it is not the identification document 10 but the person 1 (themselves) that is captured by the capture unit 31 of the terminal device 30.

For identification 201, the biometric data captured in step 2011 is compared with the biometric data of the personal data set 320 by the identification unit of the terminal device 30 in step 2012. This biometric data of the personal data set 320 can be retrieved from the storage unit 32 of the terminal device 30. This biometric data of the personal data set 320 was preferably obtained using the method 100 of FIG. 1 (system 1000 of FIG. 2). This may require decryption of the personal data set 320 (if encryption was performed in step 104 of the method 100).

Alternatively (or additionally), this biometric data may be retrieved from a background system 40 in step 2013 (for example, if it was sent there in step 106). The background system 40 could be a confidential entity for managing personal data sets 320. Retrieving the background system 40 is less preferable because it requires biometric data to be available outside the terminal 30. A comparison 2012 with locally stored biometric data is preferred for security reasons.

To identify 202 the person 1, a face 1a of the person 1 can be captured in the capturing step 2011 by the capturing unit 31, in particular a camera 31a of the terminal device 30. The captured face 1a can be analyzed by the evaluation unit 33 of the terminal device 30 using computer-aided facial recognition methods in order to obtain the biometric data of the person 1.

The identification 202 of person 1 can be repeated at one or more random times during the performance of the procedure. This ensures that the identified (and verified) person 1 performs the procedure for the entire duration of the procedure.

In a subsequent step 202, at least one object 20 to be changed by the procedure is identified by the terminal 30 immediately before the procedure is carried out. The object 20 is in particular an object that is changed by the procedure from a first state to a second state. The object 20 can be a test object, for example a test cassette of a virus detection test. The first state of this test cassette could then be that the test cassette has an unused test strip. The second state (after the procedure) could then be that the test cassette—by applying a swab, saliva or spit sample—has a used test strip. The used test strip has at least one visible control line (=control bar) compared to the unused test strip, while the unused test strip has no visible control line.

To identify 202 the object 20, a query 2023 of an object identification data set 420 or a procedure data set 45) from the (or another) background system 40 may be performed.

This query 2023 can be used to query an object identification data set 420 shown in FIG. 9c. The object identification data set 420 can, for example, comprise an object type. This is useful if a procedure can be carried out with different types of objects 20. The instruction and documentation can then be adapted accordingly using the object type. The object identification data set 420 can, for example, comprise a manufacturer specification, a production date, an expiry date, an object property and/or a certificate. This allows the object 20 to be best identified in the method 200 and useful information for performing the procedure to be provided.

This query 2023 can alternatively or additionally query a procedure data set 450 shown in FIG. 9e. The procedure data set 450 can, for example, comprise a procedure serial number.

The procedure data set 450 may, for example, comprise a status of the object 20. A status of the object 20 may be “initialized”, for example when the object was created in the procedure 200. A status of the object 20 may be “manufactured”, for example when the object was manufactured for the procedure 200. A status of the object 20 may be “in use”, for example when the object is used in the method 200. A status of the object 20 may be “completed”, for example when the object has been completed in the process 200.

The procedure data set 450 can comprise a procedure result. The procedure result can be “positive”, “negative” or “invalid”. An invalid procedure result is output if the procedure was used incorrectly or person 1 and/or object 20 could not be identified/traced in accordance with the procedure. A positive procedure result can be issued if a procedure has been completed without errors or manipulation. Based on a virus detection test as a procedure, a positive test result is the detection of viruses by the test cassette. Based on a virus detection test as a procedure, a negative test result is the lack of detection of viruses by the test cassette.

The procedure data set 450 may comprise a timestamp, for example a time or date. The procedure record 450 may comprise the scatter value 430. The procedure record 450 may comprise a procedure identification number. The procedure record 450 may comprise a person identification number.

The identification 202 of the object 20 can comprise the capture 2021 of an object identification data set 21 of the object by the terminal device 30 and a comparison 2022 of the captured object identification data set 21 with the object identification data set 420 of the background system 40. This increases security against manipulation. The object identification data set 21 is, for example, a machine-readable data set, such as a QR code 210, a barcode or an RFID/NFC tag. The machine-readable data set can also be a unique serial number 220 that was assigned in the method 200. The machine-readable data set of the object 20 can be validated by querying 2023 a corresponding object identification data set 420 in a background system 40, wherein after a successful validation of the machine-readable data set, a status of the object identification data set 420 or a procedure data set 450 in the background system 40 is set to “in use”.

In the step 202, each aid 50 can optionally also be identified by the terminal device 30 immediately before the procedure. In this case, each aid 50 used can be detected by a camera 31a of the terminal device 30. The captured assistive device 50 may be analyzed by methods of computer-aided facial and/or pattern and/or image recognition by the evaluation unit 33 of the terminal device 30 to identify the assistive device 50.

After successful identification in steps 201 and 202, the procedure is carried out by person 1. For this purpose, the person may be asked to position themselves in front of the terminal device 30 and activate the front camera 31a of the terminal device 3900 (so-called selfie mode). This allows the user to observe themselves while the procedure is being carried out (self-mirroring). This allows the user to examine themselves and the terminal device 30 can also recognize and capture the person 1, the object 20 and/or the aid 50. The capture unit 31 of the terminal device 30 remains activated throughout the procedure, allowing the terminal device 30 to capture and analyze images.

For this purpose, the person may also be requested not to leave an area that can be detected by the camera 31a of the terminal device or a predefined area.

The detectable area can be displayed on the display unit 34 of the terminal device 30 during the entire procedure or only at the beginning of the procedure. This area can be indicated by “augmented reality” methods in the display unit 34 of the terminal device 30 by lines, hatching, etc. It can be determined by distances to the camera, lighting conditions, etc. of the terminal device 30 itself. It can be predetermined by the procedure itself or the object 20 and/or the aids 50.

In a step 203, at least one position of the person 1 and at least one position of the object 20 and possibly a position of the assistive device 50 is tracked by the terminal device 30 using methods of computer-aided gesture and/or pattern and/or image recognition during the performance of the procedure. Here, positions and movements can be recorded and analyzed by the evaluation unit 33 of the terminal device using methods of computer-aided facial and/or pattern and/or image recognition. For example, the position and/or movement of a body part of the person 1, preferably one or both hands 12 of the person 1, is recorded and analyzed.

Thereby, a tracking unit of the terminal device 30 may be arranged for tracking 203 one or more positions of the person 1 during the execution of the test procedure by methods of computer-aided gesture and/or pattern and/or image recognition by checking whether both hands 12 of the person 1 are in a predefined area or an area detectable by the camera 31a of the terminal device 30 during the entire execution of the test procedure.

In this regard, a tracking unit of the terminal device 30 may be adapted to track 203 one or more positions of the test object 20 based on the shape of the object during execution of the test procedure using methods of computer-aided gesture and/or pattern and/or image recognition.

The checking by the tracking unit of the terminal device 30 may also include checking whether the face 1a and the two hands 1b of the person 1 are in the predefined area or the area detectable by the camera 31a of the terminal device 30 during a delivery of a saliva sample.

The checking by the tracking unit of the terminal device 30 may also include checking whether the face 1a and the two hands 1b of the person 1 are in the predefined area or the area detectable by the camera 31a of the terminal device 30 during an addition of an extraction liquid to the saliva sample.

The checking by the tracking unit of the terminal device 30 can also include checking whether the face 1a and the two hands 1b of the person 1 are in the predefined area or the area detectable by the camera 31a of the terminal device 30 for a predefined period of time during shaking of the saliva sample

The checking by the tracking unit of the terminal device 30 may also include checking whether the face 1a and the two hands 1b of the person 1 and the object 20 are in the predefined area or the area detectable by the camera 31a of the terminal device 30 during an injection of the saliva sample onto the object 20.

During the execution of the procedure, the procedure is instructed by instructions or warnings 204.

During procedure execution, the procedure is documented 205, for example by taking screenshots of the terminal device 30 at randomly selected times and storing them in a procedure identification data set 321 in the storage unit 32 of the terminal device 30. An exemplary structure of a procedure identification record 321 is shown in FIG. 9d. The procedure identification data set 321 of FIG. 9d comprises a procedure identification number, which is preferably unique throughout the procedure and system. The procedure identification data set 321 also comprises the procedure documentation, which comprises results and/or intermediate results and/or snapshots (screenshots) of the execution.

FIG. 4 shows the system 2000, reference is made to the explanations in FIG. 3. The system components (terminal 30, background system 40) are preferably the system components from FIG. 2 or FIG. 4; to avoid repetition, reference is made to the statements in FIGS. 1 to 3.

The evaluation unit 33 can comprise a pattern recognition module 333. This pattern recognition module 333 is set up to carry out methods of computer-aided face, pattern, character and/or image recognition. For example, a “model target tracking” method from the field of artificial intelligence is used to detect and analyze the position or movements of person 1, object 20 and/or aid 50.

For example, sensors (not shown in FIG. 4) are attached to the person 1 (e.g. to the hands 12) for detecting and analyzing the position or movements of person 1, object 20 and/or aid 50. The sensor data generated by these sensors is provided to the terminal device 30 as position or movement information and is evaluated by the evaluation unit 33 when detecting and analyzing the position or movements of person 1, object 20 and/or aid 50. For example, UWB sensors or Bluetooth sensors are used. The sensors are acceleration and/or position sensors.

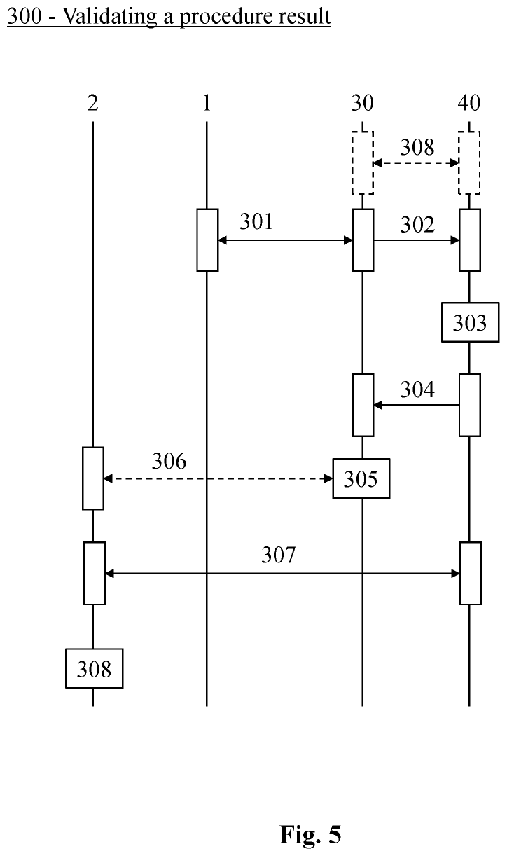

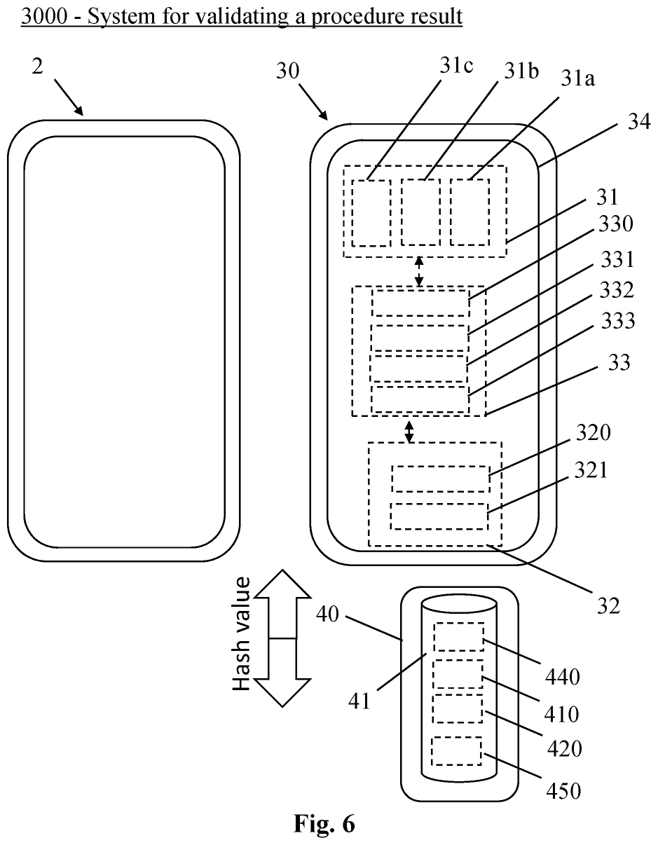

FIG. 5 shows a flowchart of a method 300 for computer-aided validation of a procedure performed by a person 1 and tracked by a computer. Elements shown in dashed lines are optional. FIG. 6 shows a system 3000 for computer-aided validation of a procedure carried out by a person 1 and tracked by a computer. In the description of the procedure 300 of FIG. 5, the system 3000 of FIG. 6 is also discussed. The procedure 300 can be directly connected to the procedure 200 of FIG. 1. An exemplary connection is shown in FIG. 8j.

In a step 301, a validation of a procedure result is requested by a person input at a terminal 30. For this purpose, the person 1 selects at the terminal 30 which procedure result is to be validated. For example, various procedure results can be selected in the terminal 30.

In step 302, a validation request is generated in the terminal 30 and sent to the background system 40. The validation request comprises the scatter value 430 via the personal data set 320 and a procedure identification data set 321 of the procedure result to be validated. Personal data set 320 and a procedure identification data set 321 are obtained accordingly from the storage unit 32 of the terminal device 30, and decryption may need to be performed if step 104 has been applied.

In the background system 40, a validation token 440 is created in step 303. An exemplary structure of a validation token is shown in FIG. 9f. The validation token 440 also comprises, for example, the scatter value 430 of the personal data set 320 (equal to the scatter value 430 of the personal data set 320 in the procedure data set 450 or to the scatter value 430 of the personal data set 320 in the anonymized personal data set 410). The validation token 440 also comprises, for example, the procedure identification number. The validation token 440 comprises token properties, for example a period of time for which the validation token 440 is valid and/or a signature of the background system 40. The validation token 440 is, for example, a jwt-token. The scatter value 430 can be created anew for each validation request and sent to the background system 40 with the procedure identification number for validating the procedure result (procedure 300). The validation then takes place via the token 440 and compares the previously transferred parameters with the database documented in the background system 40. If the data comparison is positive, the validation is positive. If the data comparison is negative (if the transferred data differs from the stored data), the validation is negative.

This validation token 440 is received in the terminal device 30 in step 304.

In display step 305, a machine-readable code with the validation token 440 and information on the background system 40 (link to the validation server) is generated and displayed together with the procedure result on the display unit 34 of the terminal device 30. A verification instance 2, for example an auditor or a machine with a scanning function, reads the machine-readable code (a quasi unconfirmed validation confirmation) in step 306. With the reading in step 306, the checking instance 2 makes a validation request to the background system 40, checking the validation token 440. If the validation token 440 is valid, the procedure result, the scatter value 430 via the personal data set 320 and preferably procedure information can be retrieved from the background system 40. The machine-readable code is displayed together with the procedure result on the display unit 34 of the terminal device 30.

A verification instance 2, for example an auditor or a machine with a scanning function, reads the machine-readable code from step 305 in step 306. With the reading in step 306, the verification instance 2 makes a validation request by checking the validation token 440. If the validation of the token 440 is valid, the background system can retrieve the procedure result, the scatter value 430 via the personal data set 320 and preferably procedure information. In a display step 308, the validation result relating to the procedure result together with the procedure result and the scatter value 430 via the person data set 320 and preferably the procedure information is displayed on a display unit of the verification instance 2.

This computer-aided tracked procedure is preferably a computer-aided tracked procedure according to the method 200 for computer-aided tracking the procedure to be performed by the person 1.

The validation token 440 has defined a validity period for the procedure result. This validity period is ten minutes, for example. This means that person 1 and test instance 2 have ten minutes after request step 301 to validate the procedure result. After this time, the validation token is invalid and validation is no longer possible.

The procedure result can be validated as often as required.

The procedure information is, for example, a procedure date and/or a procedure time according to the background system. This procedure information is displayed on the display unit of the test instance 2, whereby a user of the test instance 2 sees the information according to the background system 2 (not according to the terminal device 30). This also applies to the procedure result received from the background system and displayed in the test instance 2.

The procedure identification record 321 is generated by the terminal device 30.

The machine-readable code is a QR code. Test instance 2 therefore has a detection unit (camera, reader) to read the QR code in step 306.

FIG. 6 shows the system 3000, reference is made to the explanations in FIG. 5. The system components (terminal device 30, background system 40) are preferably the system components from FIG. 2 or FIG. 4; to avoid repetition, reference is made to the statements in FIGS. 1 to 5.

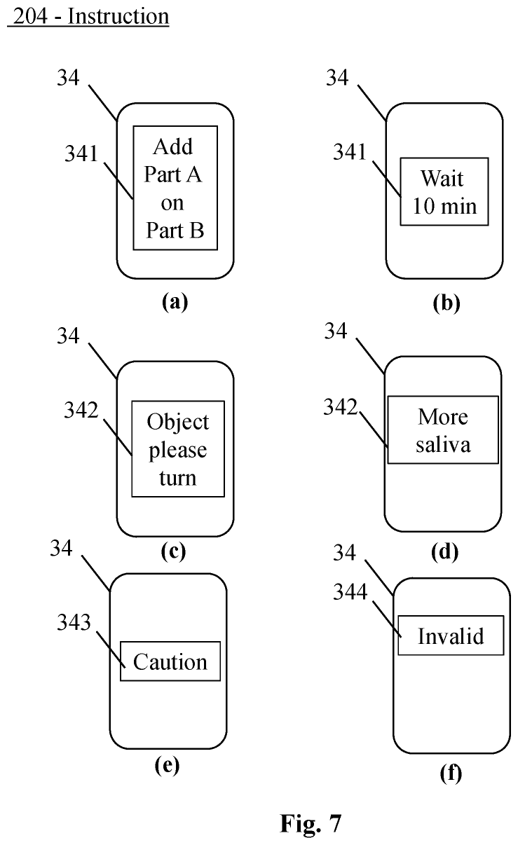

FIGS. 7a-e each show a display element 34 of a terminal device 30 on which instructions are shown during the performance of the procedure.

For example, FIG. 7a shows an instruction 341 on how the object 40 must be handled/modified in order to successfully perform a procedure. Here, a part A must be attached to a part B; this could be the assembly of a test apparatus. The (correct or incorrect) execution of this instruction 341 is then tracked by the terminal device 30.

For example, FIG. 7b shows an instruction 341 that a waiting time of 10 minutes must now be observed in order to successfully carry out a procedure. The (correct or incorrect) execution of this instruction 341 is then tracked by the terminal device 30. For example, an error could be assessed if the object 40 is removed from the predefined or detectable area of the detection unit of the terminal device 30 within this waiting time.

For example, FIG. 7c shows an instruction 342 on how a position of the object 40 must be corrected in order to successfully perform a procedure. Here, the object 40 must be rotated; this instruction 342 may have been given after recognizing an incorrect positioning of the object 40. The (correct or incorrect) execution of this instruction 342 is then tracked by the terminal device 30.

For example, FIG. 7d shows an instruction 342 as to what must be corrected on the object 40 in order to successfully perform a procedure. Here, more saliva must be applied to the object 40; this instruction 342 may have been given after recognizing that not enough saliva was supplied to the object 40. The (correct or incorrect) execution of this instruction 342 is then tracked by the terminal device 30.

For example, FIG. 7e shows a warning 343 that must be observed in order to perform a procedure successfully and/or to avoid injury. This could be a warning that a sufficient amount of extraction fluid must be applied and that the container used may be unsuitable.

For example, FIG. 7d shows an (intermediate) result 344 of a step or the entire procedure. Here, a result is considered “invalid”.

FIG. 8a-k shows a flowchart of the methods 100, 200, 300 for a virus test, in this case an antigen test for SARS-CoV-2, as an exemplary procedure. This flow chart enables the test to be performed as a spit test or as a swab test. Other test procedures are not excluded, optional steps are shown as dashed lines.

Other procedures, in particular human medical test procedures, for analyzing fluids (blood, swab, saliva, aerosol, tear fluid, sweat, sputum, urine, and/or stool) of a human or an animal, for example another virus detection test, a germ test, a bacterial test, a fungal test, etc., can also be mapped by the method according to the invention. The body fluid can be taken from a skin surface, for example from a hand or a forehead. The body fluid of a person and/or an animal is thereby analyzed for infections, properties and/or parameters.

This flowchart instructs a person 1 to perform an antigen test for SARS-CoV-2 independently and in particular without another person (via video communication or physically present). This antigen test for SARS-CoV-2 can be carried out conveniently from anywhere. This SARS-CoV-2 test fulfils all safety and certification standards and is considered as if this SARS-CoV-2 test had been accompanied, supervised or even carried out by a certified person (in particular human medical personnel such as doctors, nurses, pharmacists). In addition to instructing the person to perform the test correctly, the positions/movements of the person and the test object (in this case a test cassette or test strip) are also tracked (step 203) and these positions/movements are checked for correctness and their performance documented (step 205).

This flowchart also includes setting up a person profile (procedure 100, see FIGS. 1 and 2) and validating a procedure result (procedure 300, see FIGS. 3 and 4).

According to FIG. 8a, in step 1003 it is checked whether a personal profile (=personal data set 320) is present in the storage unit 32 of the terminal device 30.

If it is determined in step 1003 that a person profile is present (yes case), the method 100 for setting up a person profile can be skipped/skipped and it is checked in an optional subsequent step 1004 by the terminal device 30 whether (firstly) a quarantine end date of the person data set 320 is present and whether (secondly) this quarantine end date is after the current date. The current date is, for example, the date on which this procedure was carried out.

If the optional step 1004 determines that a quarantine end date is present in the person record 320 and that this quarantine end date is after the current date (yes case), the person 1 must remain in quarantine. With optional step 1005, a message is displayed on the display unit 34 of the terminal device 30 informing the person 1 that the person 1 must remain in quarantine. An antigen test for SARS-CoV-2 does not have to be carried out on this person 1, because regardless of the result of this antigen test for SARS-CoV-2, the person is not allowed to be in public. Steps 1004 and 1005 can be used to save one test cassette (as test object 20). After the notification in optional step 1005, the procedure according to FIG. 8a-k is terminated.

If it is determined in the optional step 1004 that either no quarantine end date is present in the personal data set 320 or that an existing quarantine end date is prior to the current date (no case), a start screen for selecting the execution of the method 200 (see also FIG. 3) and the method 300 (see also FIG. 5) is performed according to FIG. 8c.

If it is determined in step 1003 that no person profile exists (no case), the method 100 according to FIG. 1 (or the system 1000) is started to set up a person profile.

In an optional step 1001, the person 1 is (firstly) shown the T&Cs on the display unit 34 of the terminal device 30 and (secondly) the person 1 is shown on the display unit 34 of the terminal device 30 that these T&Cs are to be accepted.

In an optional step 1002, it is then checked whether the person 1 rejects the GTC or wishes to leave the method by making a corresponding user input on the input unit 35 of the terminal device 30.

If it is determined in the optional step 1002 that the person 1 rejects the GTC or wishes to leave the procedure (yes case) by making a corresponding user input on the input unit 35 of the terminal device 30, the procedure is terminated in accordance with FIGS. 8a-k.

If it is determined in the optional step 1002 that the person 1 does not reject the GTC or does not wish to leave the procedure (no case) by a corresponding user input on the input unit 35 of the terminal device 30, the procedure 100 is carried out according to FIG. 8b (based on FIG. 2 and FIG. 3), in particular by steps 101 and 102, see dashed rectangle.

In step 1011, the person 1 is prompted with a message on the display unit 34 of the terminal device 30 to allow access to a camera 31a of the terminal device 30 and to show a valid identification document 10 of the person 1 with front side 12 and back side 13. This message can be displayed step by step and before the next step is displayed, the person performs the respective step.

In the subsequent step 1021, the identification document 10 shown is captured by the camera 31a of the terminal device 30 (front and back in each case) and an OCR recognition is performed by the evaluation unit 33 in order to obtain the personal data 15 of the identification document 10 for the personal data set 320. In this step 1021, a photograph 14 of the identification document 10 shown in step 1011 can also be captured by the camera 31a of the terminal device 30 in order to obtain biometric data for the personal data set 320.

In the optional step 1012, the person 1 is prompted with a message on the display unit 34 of the terminal device 30 to activate the front camera 31a of the terminal device 30 and to create an image of the face 1a of the person 1 for verifying a photograph 14 of the identification document 10. In the optional subsequent step 1022, the created image of the person's face 1a may be captured by the camera 31a of the terminal device 30 to obtain biometric data for the person record 320. This obtained biometric data of the captured face 1a is first linked to the biometric data of the photograph 14 of the identification document 10.

Communication in steps 1011, 1021, 1012 and 1022 can take place via an encrypted data stream 1013 in cooperation with an external service provider. The video data and the OCR data are encrypted to secure the personal data set 320 obtained in this way. If the comparison shows that the biometric data matches with sufficient accuracy, the biometric data is accepted by the photograph 14 and provided as part of the user data set 320.

In the optional step 1023, it is then checked whether the character recognition and the photo image recognition of steps 1011, 1021, 1012 and 1022 were successful (i.e. whether a valid user data set 320 was obtained). For this purpose, the obtained biometric data of the captured face 1a is compared with the biometric data of the photograph 14 of the identification document 10 by the evaluation unit 33 of the terminal device 30.

If, in the optional step 1023, the evaluation unit 33 determines that the biometric data obtained from the captured face 1a does not match the biometric data of the photograph 14 of the identification document 10 with sufficient accuracy or that the character recognition and photograph recognition of steps 1011, 1021, 1012 and 1022 were not successful (no-case), a request to the person 1 is generated in the optional step 1024 and indicated by the display unit 34 of the terminal device 30 that another identification document is to be used or that the method 100 is to be tried again under better lighting conditions. After the notification in optional step 1024, the method according to FIG. 8a-k is terminated.

If, in the optional step 1023, the evaluation unit 33 determines that the obtained biometric data of the captured face 1a matches the biometric data of the photograph 14 of the identification document 10 with sufficient accuracy or that the character recognition and photograph recognition of steps 1011, 1021, 1012 and 1022 were successful (yes case), the user data set 320 is finalized in step 1025.

Optionally, in this step 1025, the person 1 is allowed to edit the address as a personal datum before saving 103. Step 1025 corresponds to steps 103, 104 and 105 of FIG. 1, and no further comments are made.

The result of steps 1011, 1021, 1012, 1022, 1023 and 1025 is a user data set 320 comprising biometric data and personal data 15 based on the identification document 10 (and, if applicable, the verification of the person 1 in step 1012). This user data set 320 may be stored in encrypted form in a storage unit 32 of the terminal device 30.

After step 1025, the process 200 (see also FIG. 3) and 300 (see also FIG. 5) is carried out according to FIG. 8c.

According to FIG. 8c, in optional step 199, a selection screen is shown for tracking the antigen test for SARS-CoV-2 according to method 200 and for validating the antigen test performed for SARS-CoV-2 according to method 300. With a user selection on the terminal device 30 in step 199, either method 200 or method 300 can be selected. This selection screen in step 199 is optional, the procedures 200 and 300 can also be carried out individually and independently of each other by different applications in the terminal device 30.

If the person 1 chooses to perform a new antigen test for SARS-CoV-2 in step 199, the procedure 200 is carried out as shown in FIGS. 3 and 4. For this purpose, an introductory video is displayed on the display unit 34 of the terminal device 30 in the optional step 2041 and the evaluation unit 33 of the terminal device 30 checks whether the environmental conditions (light, room) fulfil the requirements for an antigen test for SARS-CoV-2 and the requirements for this procedure 200.

If, in step 2041, the terminal device 30 determines that the environmental conditions are unsuitable for carrying out the antigen test for SARS-CoV-2 or for being able to track its performance (e.g. due to poor lighting conditions or due to a large amount of image noise or due to a lot of movement in the camera image, etc.) (abandonment case).) (exit case), the procedure result “invalid test” is displayed to person 1 in the subsequent step 2049a (in FIG. 8g), this procedure result is documented (e.g. in the procedure identification data set 321) and the procedure is ended via an “Exit” button.

If, in step 2041 (FIG. 8c), the terminal device 30 determines that the environmental conditions are suitable for performing the antigen test for SARS-CoV-2 and that its performance can be tracked (yes case), in the optional subsequent step 2042, the person 1 is shown instructions for setting up the terminal device 30 on the display unit 34 of the terminal device 30. With these instructions, the person 1 is instructed to activate the front camera 31a of the terminal device, set the correct camera perspective and position themselves in front of the terminal device 30 in such a way that the camera 31a can capture the face 1a of the person 1. In the subsequent step 2043, a check is carried out to determine whether the terminal device 30 has been set up successfully.

If, in the optional step 2043, the terminal device 30 determines that the setup of the terminal device 30 was not successful, the common selection screen is shown again in step 199 (FIG. 8c).

If, in the optional step 2043, the terminal device 30 determines that the terminal device 30 has been successfully set up, an attempt is made to identify the person 1 in step 201. For this purpose, the current camera image of the terminal device 1 is captured (i.e. the face 1a of the person 1) and compared with the biometric data of the person data set 320 (see also explanations on FIG. 3).

In the following step 2014, the terminal device 30 checks whether the identification of person 1 was successful. If it is determined in step 2014 that the identification of person 1 was not successful (abandonment case), the common selection screen is shown again in step 199 (FIG. 8c).

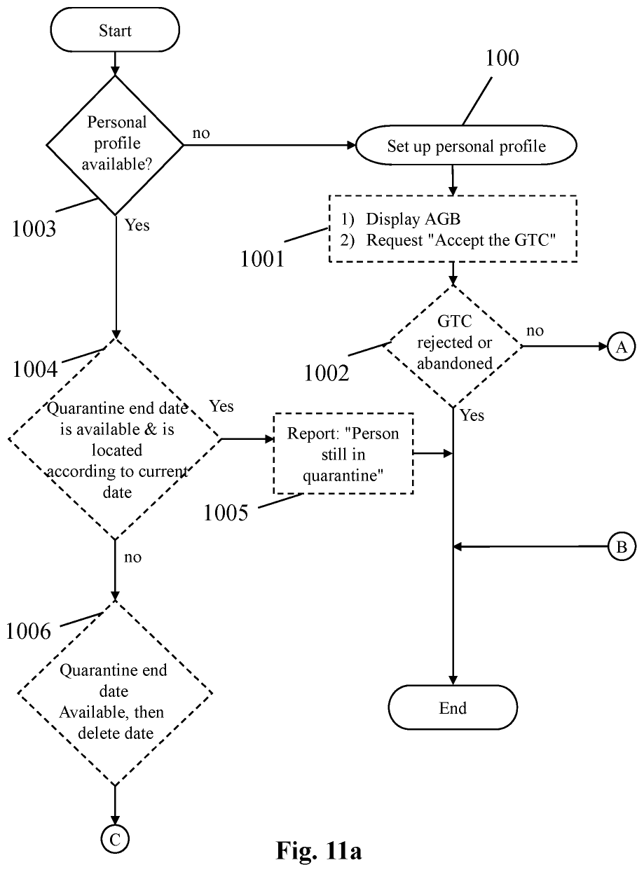

If the terminal device 30 determines in step 2014 that the identification of person 1 was successful (yes case), instructions for preparing the antigen test for SARS-CoV-2 are displayed in the optional subsequent step 2044a. This preparation can be carried out in various ways, two of which are shown in FIGS. 10a and 10b.

In the subsequent step 202 (FIG. 8d), an attempt is made to identify the test cassette 20 (the test object). A QR code 210 of the test cassette 20 is captured and it is checked whether a procedure serial number encoded in the QR code 210 matches a procedure serial number of a procedure data set 450 of the background system 40. In addition, the step 202 can check whether a status of the procedure record 450 is set to “manufactured” and whether this status can be set to the status “in use”. In addition, the step 202 can check whether the scatter value 430 of the person data set 320 of the procedure data set 450 can be assigned to the person 1.

If the terminal device 30 determines in step 202 that the identification of the test cassette 20 was not successful (no case), the person is informed in the subsequent step 2045a by a message on the display unit 34 of the terminal device 30 that the test cassette 20 of this antigen test for SARS-CoV-2 was not recognized or that the present test cassette 20 has an invalid/invalid QR code. The selection screen in step 199 is then shown (FIG. 8c).

If the terminal device 30 determines in step 202 that the identification of the test cassette 20 was successful (yes case), the test procedure can be carried out because both the person 1 and the test object 20 were identified (steps 201, 202). This means that the test procedure can now be followed up in accordance with step 203 (here specifically steps 203a-h). In the optional step 2030 of FIG. 8d, a choice can be made between various test procedures for a virus detection test, here an antigen test for SARS-CoV-2. The swab test and the spit test are described here by way of example; other tests for analyzing the body fluid of person 1 are also conceivable.

The tracking of the test procedure by the terminal device 30 is first described using the spit test in FIGS. 8d-8g. FIGS. 8j and 8k then describe the swab test (=stick test) according to step 2100.

In the optional step 2046, the person 1 is asked by the display unit 34 of the terminal device 30 whether they have eaten, drunk or smoked in the last 10 minutes.

If, in step 2046, the terminal device 30 determines (by a corresponding person input in the input unit 35 of the terminal device 30) that the person 1 has eaten, drunk or smoked in the last 10 minutes (yes case), the person 1 is informed in an optional step 2045b that the test is to be repeated at a later time. In addition, the status of the procedure record 450 in the background system 40 can be reset from the status “in use” to the status “prepared”. The selection screen in step 199 is then shown again (FIG. 8c).

If, in the optional step 2046, the terminal device 30 determines (by a corresponding person input in the input unit 35 of the terminal device 30) that the person 1 has not eaten, drunk or smoked in the last 10 minutes (no case), then in the optional step 2044b (FIG. 8e) the person 1 is instructed (firstly) to assemble the test and (secondly) to hold the assembled test in the camera 31a of the terminal device 30. The assembly can then take place at the same time as the instruction. The instruction may include an animated video. The instruction can be supported by augmented reality. During assembly, the assembly is documented according to step 205 by taking at least one picture of the person 1 during assembly by the camera 31a of the terminal device 30. Several, for example 2, images or one or more videos can also be taken by the camera 31a of the terminal device 30 of the person 1 during assembly.

These images are stored in a product documentation in the data set 321 in the storage unit 32 of the terminal device 30.

In the optional step 203a, the terminal device 30 checks whether the assembly in step 2044b was successful. For this purpose, both the face 1a and both hands 1b of the person 1 must be constantly in the predefined area (e.g. a frame of the display unit 34 or a drawn area (“augmented reality”) within the detectable area of the camera 31a, shown on the display unit 34) and must never have left this area. In addition, the assembly must correspond to the assembly of the test type (object identification data set 420), which is determined by the evaluation unit 33 of the terminal device by comparison.

If the terminal device 30 determines in the optional step 203a that the assembly was not successful (no case), the person 1 is informed in step 2045b (FIG. 8d) that the antigen test for SARS-CoV-2 must be repeated at a later time. In addition, the status of the procedure record 450 in the background system 40 can be reset from the status “in use” to the status “produced”. The selection screen in step 199 is then shown again (FIG. 8c).

If the terminal device 30 determines in step 203a that the assembly was successful (yes case), then in step 2048a (FIG. 8e) the person 1 is instructed with the display unit 34 of the terminal device 30 to dispense a saliva sample into a container as an aid 50 (up to a filling line) and to confirm with a voice input as soon as he is ready to dispense the saliva sample. From the time of the voice input, (firstly) the delivery of the saliva sample by person 1 is tracked. During the delivery of the saliva sample, according to step 205, (secondly) the delivery of the saliva sample can be documented by taking at least one picture by the camera 31a of the terminal device 30 of the person 1 during the delivery of the saliva sample. Several, for example 2, recordings or also one or more videos can also be taken by the camera 31a of the terminal device 30 of the person 1 during the saliva sample delivery. These images are stored in a product documentation in the data set 321 in the storage unit 32 of the terminal device 30.

In step 203b, the terminal device 30 checks whether the saliva sample was successfully dispensed. For this purpose, both the face 1a and both hands 1b of the person 1 must be permanently in the predefined area (e.g. in the picture frame of the display unit 34) and must never have left this area, which is determined by the evaluation unit 33 of the terminal device by tracking the position of the face 1a and the hands 1b. In addition, the saliva sample must be dispensed into the container up to the filling line (object property in the object identification data set 420), which is determined by the evaluation unit 33 of the terminal device by tracking the filling quantity of the saliva sample in the container and recognizing that the filling line of the container has been reached. The container must always be in the predefined area (e.g. in the picture frame of the display unit 34) and must never leave this area, which is detected by the evaluation unit 33 of the terminal device by tracking the container.

If the terminal device 30 determines in step 203b that the saliva sample was not successfully provided (no case), a message is generated on the display unit 34 of the terminal device 30 in step 2045c (FIG. 8f), informing the person 1 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If the terminal device 30 determines in step 203b that the saliva sample was successfully dispensed (yes case), the person 1 is instructed in step 2048b (FIG. 8e) to add an extraction liquid to the container with the saliva sample, close the container and confirm as soon as he is ready to do so. With the confirmation (voice or otherwise), the addition is (firstly) tracked. During the addition, according to step 205 (secondly) the addition is documented by taking at least one picture of the person 1 by the camera 31a of the terminal device 30 while the extraction liquid is being introduced into the container. Several, for example 2, images or also one or more videos can also be taken by the camera 31a of the terminal device 30 of the person 1 while the extraction liquid is being introduced into the container. These images are stored in a product documentation in the data set 321 in the storage unit 32 of the terminal device 30.

In step 203c, the terminal device 30 checks whether the extraction fluid has been successfully introduced into the container. For this purpose, both the face 1a and both hands 1b of the person 1 must be permanently in the predefined area (e.g. in the picture frame of the display unit 34) and must never have left this area, which is determined by the evaluation unit 33 of the terminal device by tracking the position of the face 1a and the hands 1b. The container must always be in the predefined area (e.g. in the picture frame of the display unit 34) and must never leave this area, which is determined by the evaluation unit 33 of the terminal device by tracking the position of the container.

If the terminal device 30 determines in step 203c that the addition of the extraction fluid was not successful (no case), person 1 is informed in step 2045c (FIG. 8f) that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If the terminal device 30 determines in step 203c that the addition of the extraction fluid was successful (yes case), then in step 2048c (FIG. 8f) the person 1 is instructed to shake the container with the extraction fluid and the saliva sample for 5 seconds. The shaking takes place at the same time as the instruction. During the shaking, the shaking is documented according to step 205 by taking at least one picture of the person 1 during the shaking using the camera 31a of the terminal device 30. Several, for example 2, images or one or more videos can also be taken by the camera 31a of the terminal device 30 of the person 1 during the shaking. These recordings are stored in a product documentation in the data set 321 in the storage unit 32 of the terminal device 30.

In step 203d, the terminal device 30 checks whether the shaking was successful. For this purpose, both the face 1a and both hands 1b of the person 1 must be permanently in the predefined area (e.g. in the picture frame of the display unit 34) and must never have left this area, which is determined by the evaluation unit 33 of the terminal device by tracking the position of the face 1a and the hands 1b. The shaking of the container by person 1 for at least 5 seconds is tracked. The container must be constantly in the predefined area (e.g. in the picture frame of the display unit 34) and must never leave this area, which is determined by the evaluation unit 33 of the terminal device by tracking the position of the container.

If the terminal device 30 determines in step 203d that the shaking was not successful (no case), person 1 is informed in step 2045c that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If the terminal device 30 determines in step 203d that the shaking was successful (yes case), person 1 is instructed in step 2048d to show the test cassette 20 and to drop the contents of the shaken container (the sample+extraction liquid) onto the test cassette 20. Pointing and dripping take place at the same time as the instruction. During the pointing and dripping, the pointing and dripping is documented according to step 205 by taking at least one picture by the camera 31a of the terminal device 30 of the person 1 during the pointing and dripping. Several, for example 2, images or also one or more videos can also be taken by the camera 31a of the terminal device 30 of the person 1 during the pointing and dripping. These recordings are stored in a product documentation in the data set 321 in the storage unit 32 of the terminal device 30.

In step 203e, the terminal device 30 checks whether the pointing and dripping was successful. For this purpose, both the face 1a and both hands 1b of the person 1 must be permanently in the predefined area (e.g. in the picture frame of the display unit 34) and must never have left this area. The test cassette 20 must be recognized and must be permanently in the predefined area (e.g. in the picture frame of the display unit 34) and must never leave this area.

If the terminal device 30 determines in step 203e that the pointing and dripping was not successful (no case), person 1 is informed in step 2045c that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If the terminal device 30 determines in step 203e that the pointing and dripping was successful (yes case), a waiting time of 10 minutes is displayed in step 2047 and counted down, for example as a countdown. Person 1 is informed as soon as the waiting time has elapsed.

One or each of the steps 2048a-d may optionally comprise a countdown. The countdown starts, for example, when the prompts are displayed on the display unit 34 of the terminal device 30. If the instruction according to steps 2048a-d is not followed or is followed incorrectly during this time, the person 1 is informed in step 2045d that the test (step) has failed due to a timeout. The test receives the procedure result “invalid”. This procedure result is entered in the procedure data set 450 of the background system 40. The selection screen in step 199 is then shown again (FIG. 8c).

In the subsequent step 2044c, the person 1 is requested to take a photo of the test cassette 20. In doing so, the person 1 may be asked to switch to a rear camera of the terminal device 30. This rear camera (usually) has a better resolution than the front camera used to track person 1 (steps 203a-h).

In the subsequent step 203f, the terminal device 30 (firstly) checks whether the serial number of the test cassette 20 matches that in step 202. For this purpose, the QR code 210 of the test cassette 20 is captured (based on steps 2044a and 202) and the serial number obtained therefrom is compared with that of a procedure data set 450. In addition, step 203f (secondly) checks whether one or more control bars can be recognized on the test strip of the test cassette 20. The control bar(s) is/are a security feature of the test cassette 20 that must always appear, regardless of the saliva sample.

If the terminal device 30 determines in step 203f that either the serial number of the test cassette 20 does not match the serial number in step 202 or that the control bar(s) of the antigen test for SARS-CoV-2 cannot be recognized on the test strip of the test cassette 20 (no case), a check is performed in step 203g to determine whether the serial number of the test cassette 20 matches the serial number in step 202.

If the terminal device 30 determines in step 203g that the serial number of the test cassette 20 does not match the serial number in step 202, the person 1 is informed in step 2045e that the serial number is incorrect. The test receives the procedure result “invalid”, which is entered into the procedure record 450 of the background system 40. The selection screen in step 199 is then shown again (FIG. 8c).

If the terminal device 30 determines in step 203g that the serial number of the test cassette 20 matches the serial number in step 202, the person 1 is informed in step 2045f that the control bar(s) of the test cannot be recognized on the test strip of the test cassette 20 (e.g. no color change in the expected area of the test strip). The test receives the procedure result “invalid”, which is entered in the procedure data set 450 of the background system 40. The selection screen in step 199 is then shown again (FIG. 8c).

If the terminal device 30 determines in step 203f that the serial number of the test cassette 20 matches the serial number in step 202 and that the control bar(s) of the antigen test for SARS-CoV-2 can be recognized on the test strip of the test cassette 20 (yes case), the terminal device 30 checks in step 203h whether the procedure result is “positive”.

If the terminal device 30 determines in step 203g that the procedure result is not “positive” (no case), the terminal device 30 displays the procedure result “negative” in step 2049a. This procedure result is documented, for example by setting the procedure result to “negative” in the procedure data set 450 of the background system 40, possibly also by documenting it in the procedure documentation of the data set 321, for example also by saving the photo from step 2044c in the procedure documentation of the storage unit 32 of the terminal device 30.

If the terminal device 30 determines in step 203g that the procedure result is “positive” (yes case), the procedure result “positive” is displayed by the terminal device 30 in step 2049b. This procedure result is documented, for example by setting the procedure result to “positive” in the procedure data set 450 of the background system 40, possibly also by documenting it in the procedure documentation of the data set 321, for example also by saving the photo from step 2044c in the procedure documentation of the storage unit 32 of the terminal device 30. In addition, the person 1 receives instructions on how to proceed further. In addition, a quarantine end date can be set in the personal data set 320 to a date in a fortnight from the current date. The procedure 200 (tracking) is then completed.

According to FIG. 8d, the terminal device 30 is optionally set up to track various test procedures for a virus detection test, in this case an antigen test for SARS-CoV-2, of which a test procedure is to be selected in the optional step 2030. Instead of the spit test described above, a swab test could also be selected in step 20230. FIGS. 8j and 8k now describe the tracing of the swab test (=swab test) according to step 2100. Steps 201, 202, 203e-h, 2044c, 2047, 2045c-f and 2049a-b are identical to the steps of the spit test and are not described again here.

In step 2144a (FIG. 8h), the person 1 is instructed to (firstly)unpack the test stick and (secondly) take a sample from a first (e.g. the left) nostril for a period of at least 5 seconds. Unpacking and taking the nasal sample can take place at the same time as the instruction. The instruction can include an animated video. The instruction can be supported by augmented reality. During the unpacking and removal of the nasal sample, the unpacking and removal of the nasal sample is documented according to step 205 by taking at least one picture of the person 1 by the camera 31a of the terminal device 30 during the unpacking and removal of the nasal sample. Several, for example 2, images or one or more videos can also be taken by the camera 31a of the terminal device 30 of the person 1 during the unpacking and removal of the nose sample. These images are stored in a product documentation in the data set 321 in the storage unit 32 of the terminal device 30.

In step 203i, the terminal device 30 checks whether the unpacking and removal of the nose sample in step 2144a was successful. For this purpose, both the face 1a and both hands 1b of the person 1 must be permanently in the predefined area (e.g. a frame of the display unit 34 or a drawn area (“augmented reality”) within the detectable area of the camera 31a, shown on the display unit 34) and must never have left this area. The container must be permanently in the predefined area (e.g. in the image frame of the display unit 34) and must never leave this area, which is determined by the evaluation unit 33 of the terminal device by tracking the position of the container.

If the terminal device 30 determines in step 203i that the unpacking and removal of the nose sample was not successful (no case), a message is generated on the display unit 34 of the terminal device 30 in step 2045c (FIG. 8f), informing the person 1 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If, in step 203i, the terminal device 30 determines that the unpacking and removal of the nose sample has not been carried out long enough (too-short case), a message is generated on the display unit 34 of the terminal device 30 (not shown), informing the person 1 that step 2144a is to be carried out further.

If the terminal device 30 determines in step 203i that the unpacking and removal of the nasal sample was successful (yes case), the person 1 is instructed in step 2144b (FIG. 8h) to remove a sample from a second (e.g. the right) nostril for a period of at least 5 seconds. The unpacking and removal of the nasal sample can take place at the same time as the instruction. The instruction can include an animated video. The instruction can be supported by augmented reality. During the removal of the nose sample, the removal of the nose sample is documented according to step 205 by taking at least one picture of the person 1 during the removal of the nose sample by the camera 31a of the terminal device 30. Several, for example 2, images or one or more videos can also be taken by the camera 31a of the terminal device 30 of the person 1 during the removal of the nose sample. These images are stored in a product documentation in the data set 321 in the storage unit 32 of the terminal device 30.

If the terminal device 30 determines in step 203j that taking the second nose sample was not successful (no case), a message is generated on the display unit 34 of the terminal device 30 in step 2045c (FIG. 8f), informing the person 1 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If, in step 203j, the terminal device 30 determines that the second nose sample has not been taken long enough (too-short case), a message is generated on the display unit 34 of the terminal device 30 (not shown) informing the person 1 that step 2144b still needs to be performed.

If the terminal device 30 determines in step 203j that the extraction of the second nasal sample was successful (yes case), the person 1 is instructed in step 2144c to open a container with extraction liquid and to stir and squeeze the nose stick in the container for at least 15 seconds. During the adding+stirring+squeezing, the adding+stirring+squeezing is documented according to step 205 by taking at least one picture by the camera 31a of the terminal device 30 of the person 1 during the adding+stirring+squeezing. Several, for example 2, images or one or more videos can also be taken by the camera 31a of the terminal device 30 of the person 1 during the adding+stirring+squeezing. These recordings are stored in a product documentation in the data set 321 in the storage unit 32 of the terminal device 30.

If the terminal device 30 determines in step 203k that the adding+stirring+squeezing was not successful (no case), a message is generated on the display unit 34 of the terminal device 30 in step 2045c (FIG. 8f), informing the person 1 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

If, in step 203k, the terminal device 30 determines that the adding+stirring+squeezing has not been carried out long enough (too-short case), a message is generated on the display unit 34 of the terminal device 30 (not shown), informing the person 1 that step 2144c is to be carried out further.

If the terminal 30 determines in step 203k that the adding+stirring+squeezing was successful (yes case), then in step 2144d (FIG. 8i) the person 1 is instructed to show the test cassette 20 and to drip the contents of the container (the sample+extraction liquid) onto the test cassette 20. Pointing and dripping take place at the same time as the instruction. During the pointing and dripping, the pointing and dripping are documented according to step 205 by taking at least one picture of the person 1 during the pointing and dripping using the camera 31a of the terminal device 30. Several, for example 2, images or also one or more videos can also be taken by the camera 31a of the terminal device 30 of the person 1 during the pointing and dripping. These recordings are stored in a product documentation in the data set 321 in the storage unit 32 of the terminal device 30.

The procedure is then continued with step 203e; please refer to the comments on the spit test.

If a validation of an already performed antigen test for SARS-CoV-2 (spit test or swab test) is selected in the common selection screen according to step 199 of FIG. 8c, the procedure according to FIGS. 8j and 8k is run through (button 300 in FIG. 8j).

Firstly, step 3001 compares the personal data set 320 with the person 1 (who is located in front of the terminal device 1). If the comparison in step 3001 shows that the personal data set 320 does not match the person 1 in front of the terminal device 30 (comparison of the biometric data and/or the user-related data) (no case), a message is displayed on the display unit 34 of the terminal device 30 that an unauthorized person 1 wants validation, which is not possible. The message could read “Invalid user to present the test”

If the comparison in step 3001 shows that the personal data set 320 matches the person 1 in front of the terminal device 30 (comparison of the biometric data and/or the user-related data) (yes case), a validation request is generated by the terminal device 30 in step 301. Based on this validation request, a validation token 440 is generated in the background system 40 in step 303. In addition, the properties of the token are linked to the validation token 440 in the database 41 of the background system 40. The validation token 440 is transmitted to the terminal device 30 in step 304.

Based on the validation token 440 received, a QR code is created in step 305, which is linked to the properties of the token. For example, this QR code is only valid for a certain period of time (e.g. 10 minutes). On the display unit 34 of the terminal device 30, the procedure result is displayed using the data set 321, possibly with the procedure documentation. With the displayed procedure documentation shown on the display unit 34 in the procedure 300, the evaluation unit 33 of the terminal device 30 can randomly select the images and/or videos recorded during the tracking procedure 200 and show them to the verifier 2 on request.

The QR code and the procedure result are displayed to a test instance in step 306. This display is visual. For this purpose, the validation instance 2 reads the QR code using a camera or a reader of the validation instance, for example. Step 307a checks whether the validation token 440 is valid. If the check in step 307a shows that the validation token 440 is not valid (no case), a message is generated on the display unit of the verifier 2 in step 308a, indicating that the validation token 440 is invalid or that the validation token 440 was queried outside the time period.

If the check in step 307a shows that the validation token 440 is valid (yes case), then the check instance 2 checks in step 307b whether the scatter value 430 of the personal data set 320 is valid and whether the procedure identification number of the procedure data set 450 is valid and whether the procedure identification number of the procedure data set 450 is linked to the scatter value 430 (i.e. whether the procedure data set 450 has the scatter value 430).

If the check in step 307b shows that the scatter value 430 of the personal data set 320 is valid and that the procedure identification number of the procedure data set 450 is valid and that the procedure data set 450 has the scatter value 430 (yes case), then in step 308c it is displayed at the test instance 2, that the test result is validated (validation result of the procedure result is positive) and the procedure data set 450 is displayed, preferably also a time stamp (time and date) of the antigen test for SARS-CoV-2 or a status of the antigen test for SARS-CoV-2 is displayed as procedure information, more preferably the test result of the antigen test for SARS-CoV-2 according to background system 40.

Corresponding units of the systems 1000, 2000, 3000 of FIGS. 2, 4 and 6 are set up to carry out these process steps. The interaction of the systems 1000, 2000, 3000 according to FIGS. 2, 4 and 6 is now described again with reference to FIGS. 8a-i.

According to FIG. 8a, the evaluation unit 33 of the terminal device 30 is set up in step 1003 to check whether a personal profile (=personal data set 320) is present in the storage unit 32 of the terminal device 30.

If the evaluation unit 33 of the terminal device 30 determines in step 1003 that a person profile is present in the storage unit 32 of the terminal device 30 (yes case), the evaluation unit 33 of the terminal device 30 is set up to skip/skip the system 1000 for setting up a person profile.

In an optional step 1004, the evaluation unit 33 of the terminal device 30 is set up to check whether (firstly) an end-of-quarantine date is present in the personal data set 320 of the storage unit 32 of the terminal device 30 and whether (secondly) this end-of-quarantine date is after the current date. The current date is, for example, the date on which this procedure was performed.

The evaluation unit 33 of the terminal device 30 is set up to determine in the optional step 1004 that a quarantine end date is present in the personal data set 320 and that this quarantine end date is after the current date (yes case). In this Yes case, person 1 must remain in quarantine. The display unit 34 of the terminal device is then set up to display a message on the display unit 34 of the terminal device 30 in the optional step 1005. This message in the optional step 105 indicates to the person 1 that the person 1 must remain in quarantine. An antigen test for SARS-CoV-2 does not have to be performed on this person 1, because regardless of the result of this antigen test for SARS-CoV-2, the person is not allowed to be in public. With the optional steps 1004 and 1005, a test cassette (as test object 20) can be saved. After the display unit 34 of the terminal device has displayed the message in the optional step 1005, the evaluation unit of the terminal device 30 is set up to end the procedure according to FIG. 8a-k.

The evaluation unit 33 of the terminal device 30 is set up to determine in the optional step 1004 that either no quarantine end date is present in the personal data set 320 or that an existing quarantine end date is before the current date (no case). In this “no” case, the display unit 34 of the terminal device is set up to display a start screen. The terminal device 30 is then set up to record a user input from the person 1, with which the execution of the procedure 200 (see also FIG. 3) and/or the procedure 300 (see also FIG. 5) is selected in accordance with FIG. 8c.

If the evaluation unit 33 of the terminal device 30 determines in step 1003 that no person profile is present in the storage unit 32 of the terminal device 30 (no case), the evaluation unit 33 of the terminal device 30 is set up to perform the method 100, which is carried out with the system 1000 for setting up a person profile.

In an optional step 1001, the display unit 34 of the terminal device is adapted to (firstly) display the GTC to the person 1 on the display unit 34 of the terminal device 30 and (secondly) indicate to the person 1 on the display unit 34 of the terminal device 30 that these GTC are to be accepted.

In the optional step 1002, the evaluation unit 33 of the terminal device 30 is set up to check whether the person 1 rejects the GTC or wishes to leave the method by a corresponding user input on the input unit 35 of the terminal device 30. The evaluation unit 33 of the terminal device 30 is set up to determine in the optional step 1002 that the person 1 rejects the GTC or wishes to leave the procedure (yes case) by making a corresponding user input on the input unit 35 of the terminal device 30. If this user input is detected, the method according to FIG. 8a-k is terminated.

The evaluation unit 33 of the terminal device 30 is set up to determine in the optional step 1002 that the person 1 does not reject the GTC or does not wish to leave the procedure (no case) by a corresponding user input on the input unit 35 of the terminal device 30. The evaluation unit 33 of the terminal device 30 is then set up to execute the method 100 based on FIG. 1 or the system 1000 based on FIG. 2, in particular by steps 101 and 102, see dashed rectangle.

The evaluation unit 33 of the terminal device 30 is configured to display a message on the display unit 34 of the terminal device 30 in step 1011, with which the person 1 is requested to allow access to a camera 31a of the terminal device 30 and to show a valid identification document 10 of the person 1 with front side 12 and back side 13. This message can be displayed step by step and before the next step is displayed, the person performs the respective step.

The capture unit 31 (here a camera 31a) of the terminal device 30 is set up to capture the identification document 10 shown in the subsequent step 1021 (front and back in each case) and the evaluation unit 33 is set up to perform OCR recognition on the captured identification document 10 in order to obtain the personal data 15 of the identification document 10 for the personal data set 320. In this step 1021, the camera 31a of the terminal device 30 may also be adapted to capture a photograph 14 of the identification document 10 shown in step 1011 in order to obtain biometric data for the personal data set 320.

In the optional step 1012, the evaluation unit 33 of the terminal device 30 is set up to request the person 1 with a message on the display unit 34 of the terminal device 30 to activate the front camera 31a of the terminal device 30 and to create an image of the face 1a of the person 1 for verifying a photograph 14 of the identification document 10. The camera 31a of the terminal device 30 is set up to capture the face 1a of the person in the optional subsequent step 1022 in order to obtain biometric data for the personal data set 320. The evaluation unit 33 of the terminal device 30 is set up to first link this biometric data obtained from the captured face 1a with the biometric data of the photograph 14 of the identification document 10.

The terminal device 30 is set up to carry out communication in steps 1011, 1021, 1012 and 1022 via an encrypted data stream 1013 in cooperation with an external service provider. The video data and the OCR data are encrypted to secure the personal data set 320 obtained in this way. If the comparison shows that the biometric data matches with sufficient accuracy, the biometric data is accepted by the photograph 14 and provided as part of the user data set 320.

The evaluation unit 33 of the terminal device 30 is set up to check in the optional step 1023 whether the character recognition and the photo image recognition of steps 1011, 1021, 1012 and 1022 were successful (i.e. whether a valid user data set 320 was obtained). The evaluation unit 33 of the terminal device 30 is configured to compare the obtained biometric data of the captured face 1a with the biometric data of the photograph 14 of the identification document 10.

The evaluation unit 33 of the terminal device 30 is configured to determine in the optional step 1023 that the obtained biometric data of the captured face 1a does not match the biometric data of the photograph 14 of the identification document 10 with sufficient accuracy or that the character recognition and photograph recognition of steps 1011, 1021, 1012 and 1022 were not successful (no case), In this “no” case, the evaluation unit 33 of the terminal device 30 is set up to generate a request to the person 1 in the optional step 1024 and to indicate to the person 1 via the display unit 34 of the terminal device 30 that a different identification document is to be used or that the procedure 100 is to be tried again under better lighting conditions (the system 1000 must be better illuminated). After displaying the message in the optional step 1024, the terminal device 30 is configured to terminate the method according to FIGS. 8a-k.

The evaluation unit 33 of the terminal device 30 is set up to determine, in the optional step 1023, that the biometric data obtained from the captured face 1a matches the biometric data of the photograph 14 of the identification document 10 with sufficient accuracy or that the character recognition and photograph recognition of steps 1011, 1021, 1012 and 1022 were successful (yes case). In this yes case, the evaluation unit 33 of the terminal device 30 is set up to finalize the user data set 320 in step 1025. Optionally, the person 1 is permitted in this step 1025 to edit the address as personal data before saving 103. Step 1025 corresponds to steps 103, 104 and 105 of FIG. 1, and no further comments are made.

The result of steps 1011, 1021, 1012, 1022, 1023 and 1025 is a user data set 320 comprising biometric data and personal data 15 based on the identification document 10 (and, if applicable, the verification of the person 1 in step 1012). This user data set 320 may be stored in encrypted form in a storage unit 32 of the terminal device 30.

According to FIG. 8c, in the optional step 199, the evaluation unit 33 of the terminal device 30 is adapted to display a selection screen for tracking the antigen test for SARS-CoV-2 according to method 200 and for validating the antigen test performed for SARS-CoV-2 according to method 300 on the display unit 34 of the terminal device 30. In the optional step 199, the evaluation unit 33 of the terminal device 30 is set up to detect a user selection at a user input unit 35 of the terminal device 30, whereupon either the method 200 or the method 300 is selected.

If the person 1 chooses to perform a new antigen test for SARS-CoV-2 in step 199, the procedure 200 is carried out as shown in FIGS. 3 and 4. The evaluation unit 33 of the terminal device 30 is then set up to display an introductory video on the display unit 34 of the terminal device 30 in the optional step 2041. The evaluation unit 33 of the terminal device 30 is set up to check whether the environmental conditions (light, room) fulfil the requirements for an antigen test for SARS-CoV-2 and the requirements for this method 200.

If, in step 2041, the evaluation unit 34 of the terminal device 30 determines that the environmental conditions are unsuitable for performing the antigen test for SARS-CoV-2 or for being able to track its performance (e.g. due to poor lighting conditions or due to a large amount of image noise or due to a lot of movement in the camera image or etc.) (abandonment case), the evaluation unit 34 of the terminal device 30 is set up in the subsequent step 2049a (in FIG. 8g) to provide the person 1 with the procedure result “invalid test”. In the subsequent step 2049a (in FIG. 8g), the evaluation unit 34 of the terminal device 30 is set up to display the procedure result “invalid test” to the person 1 on the display unit 35, to document this procedure result in the storage unit 32 of the terminal device (e.g. in the procedure identification data set 321) and to terminate the system 2000 via an “Exit” button.

If, in step 2041, the evaluation unit 33 of the terminal device 30 determines that the environmental conditions are suitable for performing the test and that its performance can be tracked (yes case), the evaluation unit 34 of the terminal device 30 is set up in the optional subsequent step 2042 to display instructions for setting up the terminal device 30 to the person 1 on the display unit 34 of the terminal device 30. Here, the front camera 31a of the terminal device is activated by a user input in order to set the correct camera perspective and to position itself in front of the terminal device 30 in such a way that the camera 31a can capture the face 1a of the person 1. In the subsequent step 2043, the evaluation unit 33 of the terminal device 30 is set up to determine whether the setup of the terminal device 30 was successful.

In the optional step 2043, the evaluation unit 33 of the terminal device 30 is set up to determine that the setup of the terminal device 30 was not successful. The evaluation unit 34 of the terminal device 30 is then set up to show the common selection screen again in step 199 (FIG. 8c).

In the optional step 2043, the evaluation unit 33 of the terminal device 30 is set up to determine that the setup of the terminal device 30 was successful. The evaluation unit 33 of the terminal device 30 is then set up to attempt to identify the person 1 in step 201. The evaluation unit 33 of the terminal device 30 is set up to capture the current camera image of the terminal device 1 (i.e. the face 1a of the person 1) and to compare it with the biometric data of the person data set 320 (see also comments on FIG. 3).

In the following step 2014, the evaluation unit 33 of the terminal device 30 is set up to check whether the identification of the person 1 was successful. If this check by the evaluation unit in step 2014 shows that the identification of person 1 was not successful (abandonment case), the evaluation unit 34 of the terminal device 30 is set up to show the common selection screen again in step 199 (FIG. 8c).

In step 2014, the evaluation unit 33 of the terminal device 30 is set up to determine that the identification of the person 1 was successful (yes case). Then, in the optional subsequent step 2044a, the evaluation unit 33 of the terminal device 30 is set up to display instructions for preparing the test on a display unit 33. This preparation can be carried out in various ways, two of which are shown in FIGS. 10a and 10b.

In the subsequent step 202 (FIG. 8d), the evaluation unit 33 of the terminal device 30 is set up to attempt to identify the test cassette 20 (the test object). Here, evaluation unit 34 of the terminal device 30 is set up to detect a QR code 210 of the test cassette 20 by the detection unit 31 of the terminal device 30 and to check whether a procedure serial number encoded in the QR code 210 matches a procedure serial number of a procedure data set 450 of the background system 40. In addition, the evaluation unit 33 of the terminal device 30 is set up in the step 202 to check whether a status of the procedure data set 450 is set to “produced” and whether this status can be set to the status “in use”. In addition, it can be checked in the step 202 whether the scatter value 430 of the person data set 320 of the procedure data set 450 can be assigned to the person 1.

In step 202, the evaluation unit 33 of the terminal device 30 is set up to determine that the identification of the test cassette 20 was not successful (no case). The evaluation unit 34 of the terminal device 30 is then set up to inform the person in the subsequent step 2045a by a message on the display unit 34 of the terminal device 30 that the test cassette 20 of this antigen test for SARS-CoV-2 was not recognized or that the present test cassette 20 has an invalid/invalid QR code. The selection screen in step 199 is then shown (FIG. 8c).

In step 202, the evaluation unit 33 of the terminal device 30 is set up to determine that the identification of the test cassette 20 was successful (yes case). The test procedure can then be carried out because both the person 1 and the test object 20 have been identified (steps 201, 202). This means that the test procedure can now be followed up in accordance with step 203 (here specifically steps 203a-h). In the optional step 2030 of FIG. 8d, the terminal device 30 is set up to track various test procedures for a virus detection test, in this case an antigen test for SARS-CoV-2, of which one test procedure is to be selected. The swab test and the spit test are described here by way of example; other tests for analyzing the body fluid of person 1 are also conceivable.

The tracking of the test procedure by the terminal device 30 is first described using the spit test in FIGS. 8d-8g. FIGS. 8j and 8k then describe the swab test (=stick test) according to step 2100.

In the optional step 2046, the evaluation unit 33 of the terminal device 30 is set up to ask the person 1 by the display unit 34 of the terminal device 30 whether they have eaten, drunk or smoked in the last 10 minutes.

In the optional step 2046, the evaluation unit 33 of the terminal device 30 is set up to determine that the person 1 has eaten, drunk or smoked in the last 10 minutes (yes case) by a corresponding person input in the input unit 35 of the terminal device 30. Then, in the optional step 2045b, the evaluation unit 33 of the terminal device 30 is set up to inform the person 1 by the display unit 34 of the terminal device 30 that the test is to be repeated at a later time. In addition, the status of the procedure record 450 in the background system 40 can be reset from the status “in use” to the status “produced”. The selection screen in step 199 is then shown again (FIG. 8c).

In the optional step 2046, the evaluation unit 33 of the terminal device 30 is set up to determine, by a corresponding person input in the input unit 35 of the terminal device 30, that the person 1 has not eaten, drunk or smoked in the last 10 minutes (no case). Then, in the optional step 2044b (FIG. 8e), the evaluation unit 33 of the terminal device 30 is set up to instruct the person 1 (firstly) to assemble the test and (secondly) to hold the assembled test in the camera 31a of the terminal device 30. The assembly can then take place at the same time as the instruction. The instruction may be an animated video on the display unit 34 of the terminal 30. The instruction can be supported by augmented reality. During assembly, the terminal device 30 is set up according to step 205 to document the assembly by setting up the evaluation unit 33 of the terminal device to capture at least one image of the person 1 by the camera 31a of the terminal device 30 during assembly. Several, for example 2, images or one or more videos can also be captured by the camera 31a of the terminal device 30 of the person 1 during assembly. The evaluation unit 33 of the terminal device is set up to store the images in a product documentation in the data set 321 in the storage unit 32 of the terminal device 30.

In the optional step 203a, the evaluation unit 33 of the terminal device 30 is set up to check whether the assembly in step 2044b was successful. For this purpose, both the face 1a and both hands 1b of the person 1 must be constantly in the predefined area (e.g. a frame of the display unit 34 or a drawn area (“augmented reality”) within the detectable area of the camera 31a, shown on the display unit 34) and must never have left this area. In addition, the assembly must correspond to the assembly of the test type (object identification data set 420), which is determined by the evaluation unit 33 of the terminal device by comparison.

In the optional step 203a, the evaluation unit 33 of the terminal device 30 is set up to determine that the assembly was not successful (no case). Then, in step 2045b (FIG. 8d), the evaluation unit 33 of the terminal device 30 is set up to inform the person 1 via the display unit 34 of the terminal device 30 that the antigen test for SARS-CoV-2 must be repeated at a later time. In addition, the status of the procedure data set 450 in the background system 40 can be reset from the status “in use” to the status “produced”. The selection screen in step 199 is then shown again (FIG. 8c).

In the optional step 203a, the evaluation unit 33 of the terminal device 30 is set up to determine that the assembly was successful (yes case). Then, in step 2048a (FIG. 8e), the evaluation unit 33 of the terminal device 30 is set up to instruct the person 1 with the display unit 34 of the terminal device 30 to dispense a saliva sample into a container as an aid 50 (up to a filling line) and to confirm with a voice input as soon as he is ready to dispense the saliva sample. From the time of the voice input, (firstly) the dispensing of the saliva sample by person 1 is recorded by the recording unit and analyzed (=tracked) by the evaluation unit 33. During the delivery of the saliva sample, the terminal device 30 is set up in accordance with step 205 to document the assembly in that the evaluation unit 33 of the terminal device is set up to capture at least one image by the camera 31a of the terminal device 30 of the person 1 during the assembly. Several, for example 2, images or one or more videos can also be captured by the camera 31a of the terminal device 30 of the person 1 during the delivery of the saliva sample. The evaluation unit 33 of the terminal device is set up to store the recordings in a product documentation in the data set 321 in the storage unit 32 of the terminal device 30.

In step 203b, the evaluation unit 33 of the terminal device 30 is set up to check whether the saliva sample was successfully dispensed. For this purpose, both the face 1a and both hands 1b of the person 1 must be permanently in the predefined area (e.g. in the picture frame of the display unit 34) and must never have left this area, which is determined by the evaluation unit 33 of the terminal device by tracking the position of the face 1a and the hands 1b. In addition, the saliva sample must be dispensed into the container up to the filling line (object property in the object identification data set 420), which is determined by the evaluation unit 33 of the terminal device by tracking the filling quantity of the saliva sample in the container and recognizing that the filling line of the container has been reached. The container must always be in the predefined area (e.g. in the picture frame of the display unit 34) and must never leave this area, which is detected by the evaluation unit 33 of the terminal device by tracking the container.

In step 203b, the evaluation unit 33 of the terminal device 30 is set up to determine that the delivery of the saliva sample was not successful (no case). Then, in step 2045c (FIG. 8f), the evaluation unit 33 of the terminal device 30 is set up to generate a message on the display unit 34 of the terminal device 30 informing the person 1 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

In step 203b, the evaluation unit 33 of the terminal device 30 is set up to determine that the delivery of the saliva sample was successful (yes case). Then, in step 2048b (FIG. 8e), the evaluation unit 33 of the terminal device 30 is set up to instruct the person 1 to introduce an extraction liquid into the container with the saliva sample, to close the container and to confirm as soon as he is ready to do so. With the confirmation (voice or otherwise), the addition is (firstly) tracked. During the addition, according to step 205, (secondly) the addition is documented by taking at least one picture by the camera 31a of the terminal device 30 of the person 1 during the introduction of the extraction liquid into the container. Several, for example 2, images or also one or more videos can also be taken by the camera 31a of the terminal device 30 of the person 1 during the introduction of the extraction liquid into the container. These images are stored in a product documentation in the data set 321 in the storage unit 32 of the terminal device 30.

The evaluation unit 33 of the terminal device 30 is set up in step 203c to check whether the introduction of the extraction fluid into the container was successful. For this purpose, both the face 1a and both hands 1b of the person 1 must be permanently in the predefined area (e.g. in the picture frame of the display unit 34) and must never have left this area, which is determined by the evaluation unit 33 of the terminal device by tracking the position of the face 1a and the hands 1b. The container must always be in the predefined area (e.g. in the picture frame of the display unit 34) and must never leave this area, which is determined by the evaluation unit 33 of the terminal device by tracking the position of the container.

In step 203c, the evaluation unit 33 of the terminal device 30 is set up to determine that the addition of the extraction liquid was not successful (no case). Then, in step 2045c (FIG. 8f), the evaluation unit 33 of the terminal device 30 is set up to inform the person 1 by the display unit 34 of the terminal device 30 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

The evaluation unit 33 of the terminal device 30 is set up in step 203c to determine that the addition of the extraction liquid was successful (yes case), then the evaluation unit 33 of the terminal device 30 is set up in step 2048c (FIG. 8f) to instruct the person 1 to shake the container with the extraction liquid and the saliva sample for 5 seconds. The shaking takes place at the same time as the instruction. During the shaking, the shaking is documented according to step 205 by taking at least one picture of the person 1 during the shaking using the camera 31a of the terminal device 30. Several, for example 2, images can also be taken by the camera 31a of the terminal device 30 of the person 1 during the shaking. These images are stored in a product documentation in the data set 321 in the storage unit 32 of the terminal device 30.

In step 203d, the evaluation unit 33 of the terminal device 30 is set up to check whether the shaking was successful. For this purpose, both the face 1a and both hands 1b of the person 1 must be permanently in the predefined area (e.g. in the picture frame of the display unit 34) and must never have left this area, which is determined by the evaluation unit 33 of the terminal device by tracking the position of the face 1a and the hands 1b. The shaking of the container by person 1 for at least 5 seconds is tracked. The container must be constantly in the predefined area (e.g. in the picture frame of the display unit 34) and must never leave this area, which is determined by the evaluation unit 33 of the terminal device by tracking the position of the container.

In step 203d, the evaluation unit 33 of the terminal device 30 is set up to determine that the shaking was not successful (no case). Then, in step 2045c, the evaluation unit 33 is set up to inform the person 1 by the display unit 34 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

In step 203d, the evaluation unit 33 of the terminal device 30 is set up to determine that the shaking was successful (yes case). Then, in step 2048d, the evaluation unit 33 is set up to instruct the person 1 by the display unit 34 to show the test cassette 20 and to drip the contents of the shaken container (the sample+extraction liquid) onto the test cassette 20. Pointing and dripping take place simultaneously with the instruction. During the pointing and dripping, the pointing and dripping is documented according to step 205 by taking at least one picture by the camera 31a of the terminal device 30 of the person 1 during the pointing and dripping. Several, for example 2, images or also one or more videos can also be taken by the camera 31a of the terminal device 30 of the person 1 during the pointing and dripping. These recordings are stored in a product documentation in the data set 321 in the storage unit 32 of the terminal device 30.

In step 203e, the evaluation unit 33 of the terminal device 30 is set up to check whether the pointing and dripping was successful. For this purpose, both the face 1a and both hands 1b of the person 1 must be permanently in the predefined area (e.g. in the picture frame of the display unit 34) and must never have left this area. The test cassette 20 must be recognized and must be permanently in the predefined area (e.g. in the picture frame of the display unit 34) and must never leave this area.

In step 203e, the evaluation unit 33 of the terminal device 30 is set up to determine that the pointing and dripping was not successful (no case). Then, in step 2045c, the evaluation unit 33 is set up to inform the person 1 via the display unit 34 that the test has failed due to violation of the test regulations or due to cancellation. The test receives the procedure result “invalid”. This procedure result is entered in the procedure data set 450 of the background system 40. The selection screen in step 199 is then shown again (FIG. 8c).

In step 203e, the evaluation unit 33 of the terminal device 30 is set up to determine that the pointing and dripping was successful (yes case). Then, in step 2047, the evaluation unit 33 is set up to display a waiting time of 10 minutes on the display unit 34 and count down, for example, as a countdown. Person 1 is informed as soon as the waiting time has elapsed.

One or each of the steps 2048a-d may optionally comprise a countdown. The countdown starts, for example, when the prompts are displayed on the display unit 34 of the terminal device 30. If the instruction after steps 2048a-d is not followed or is followed incorrectly during this time, the person 1 is informed in step 2045d that the test (step) has failed due to a timeout. The test receives the procedure result “invalid”. This procedure result is entered in the procedure data set 450 of the background system 40. The selection screen in step 199 is then shown again (FIG. 8c).

In step 2044c, the evaluation unit 33 of the terminal device 30 is set up to request the person 1 to take a photo of the test cassette 20 by the display unit 34. In doing so, the person 1 can be requested to switch to a rear camera of the terminal device 30. This rear camera (usually) has a better resolution than the front camera used to track the person 1 (steps 203a-h). The detection unit 31 of the terminal device 30 is set up to detect the test cassette 20.

In step 203f, the evaluation unit 33 of the terminal device 30 is set up to firstly check whether the serial number of the test cassette 20 matches that in step 202. For this purpose (based on steps 2044a and 202), the QR code 210 of the test cassette 20 is captured and the serial number obtained therefrom is compared with that of an object data set 450. In addition, step 203f (secondly) checks whether one or more control bars can be recognized on the test strip of the test cassette 20. The control bar or bars are one or more security features of the test cassette 20 that always occur, regardless of the saliva sample.

In step 203f, the evaluation unit 33 of the terminal device 30 is set up to determine that either the serial number of the test cassette 20 does not match the serial number in step 202 or that the control bar(s) of the antigen test for SARS-CoV-2 cannot be recognized on the test strip of the test cassette 20 (no case). The evaluation unit 33 of the terminal device 33 is then set up to check in step 203g whether the serial number of the test cassette 20 matches that in step 202.

If the terminal device 30 determines in step 203g that the serial number of the test cassette 20 does not match the serial number in step 202, the person 1 is informed in step 2045e that the serial number is incorrect. The test receives the procedure result “invalid”, which is entered into the procedure record 450 of the background system 40. The selection screen in step 199 is then shown again (FIG. 8c).

In step 203g, the evaluation unit 33 of the terminal device 30 is set up to determine that the serial number of the test cassette 20 matches the serial number in step 202. Then, in step 2045f, the evaluation unit 33 of the terminal device 30 is set up to inform the person 1 by the display unit 34 that the control bar(s) of the test cannot be recognized on the test strip of the test cassette 20 (e.g. no color change in the expected area of the test strip). The test receives the procedure result “invalid”, which is entered in the procedure data set 450 of the background system 40. The selection screen in step 199 is then shown again (FIG. 8c).

In step 203g, the evaluation unit 33 of the terminal device 30 is set up to determine that the serial number of the test cassette 20 matches the serial number in step 202 and that the control bar(s) of the antigen test for SARS-CoV-2 can be recognized on the test strip of the test cassette 20 (yes case). Then, in step 203h, the terminal device 30 checks whether the procedure result is “positive”.

In step 203h, the evaluation unit 33 of the terminal device 30 is set up to determine that the procedure result is not “positive” (no case). Then, in step 2049a, the evaluation unit 33 is set up to display the procedure result “negative” on the display unit 34. This procedure result is documented, for example by setting the procedure result to “negative” in the procedure data set 450 of the background system 40, possibly also by documenting it in the procedure documentation of the data set 321, for example also by saving the photo from step 2044c in the procedure documentation of the storage unit 32 of the terminal device 30.

In step 203h, the evaluation unit 33 of the terminal device 30 is set up to determine that the procedure result is “positive” (yes case). Then, in step 2049a, the evaluation unit 33 is set up to display the “positive” procedure result on the display unit 34. This procedure result is documented, for example by setting the procedure result to “positive” in the procedure data set 450 of the background system 40, possibly also by documenting it in the procedure documentation of the data set 321, for example also by saving the photo from step 2044c in the procedure documentation of the storage unit 32 of the terminal device 30. In addition, the person 1 receives instructions on how to proceed further.

In addition, a quarantine end date can be set in the personal data set 320 to a date in a fortnight from the current date. The procedure 200 (tracking) is then completed.

The display unit 34 of the terminal device 30 can display on the common selection screen the follow-up of an antigen test for SARS-CoV-2 according to procedure 200 (system 2000) and the validation of an antigen test already performed for SARS-CoV-2 according to procedure 300 (system 3000) for selection by the person 1. The evaluation unit 33 of the terminal device is set up to recognize the selection (button 300 in FIG. 8j) of the person with the user input 35 of the terminal device for carrying out a validation of an antigen test for SARS-CoV-2 that has already been carried out. The evaluation unit 33 of the terminal device is then set up to run through the procedure according to FIGS. 8j and 8k.

The evaluation unit 33 of the terminal device is set up to compare the personal data set 320 with the person 1 (who is located in front of the terminal device 1) in step 3001. For this purpose, biometric data is obtained, as described several times above. The evaluation unit 33 of the terminal device is set up to determine during the comparison in step 3001 that the personal data set 320 does not match the person 1 in front of the terminal device 30 (comparison of the biometric data and/or the user-related data) (no case). In this “no” case, the evaluation unit 33 of the terminal device 1 is set up to display a message on the display unit 34 of the terminal device 30. The message could read “Invalid user to present the test” to indicate that an unauthorized person 1 wants validation.

The evaluation unit 33 of the terminal device is set up to determine during the comparison in step 3001 that the personal data set 320 matches the person 1 in front of the terminal device 30 (comparison of the biometric data and/or the user-related data) (yes case). In this yes case, the evaluation unit 33 of the terminal device 1 is set up to generate a validation request in step 301 and send it to the background system 40. The background system 40 is set up to receive the validation request and to generate a validation token 440 in step 303 on the basis of this validation request. The background system 40 links the properties of the token to the validation token 440. The background system 40 is configured to transmit the validation token 440 to the terminal device 30 in step 304.

The terminal device 30 is set up to receive the validation token 440 from the background system 40. The evaluation unit 33 of the terminal device 30 is set up to create a QR code based on the received validation token 440 in step 305, which is linked to the properties of the token. For example, this QR code is only valid for a certain period of time (e.g. 10 minutes). The evaluation unit 33 of the terminal device 30 is setup to display the procedure result on the display unit 34 of the terminal device 30 using the data set 321, possibly with the procedure documentation. In the displayed procedure documentation, which is displayed on the display unit 34 in the system 300, the evaluation unit 33 of the terminal device 30 is set up to randomly select the images and/or videos recorded during the tracking procedure 200 and to show this selection to the inspection instance 2 on request.

The evaluation unit 33 of the terminal device 30 is set up to display the QR code and the procedure result of the test instance in step 306 on the display unit 34 of the terminal device 30. This display is visual. For this purpose, the test instance 2 is set up to read the QR code using a camera or a reader of the test instance.

In step 307a, the verifier 2 is set up to check whether the validation token 440 is valid. In step 307a, the validation instance 2 is set up to determine during the check in step 307a that the validation token 440 is not valid (no case), whereupon the validation instance 2 is set up to generate a message in step 308a on the display unit of the validation instance 2 indicating that the validation token 440 is invalid or that the validation token 440 was queried outside the time period.

The verifier 2 is configured in step 307a to determine during the verification in step 307a that the validation token 440 is not valid (no-case), whereupon the verifier is configured to verify in step 307b whether the scatter value 430 of the personal data set 320 is valid and whether the procedure identification number of the procedure data set 450 is valid and whether the procedure identification number of the procedure data set 450 is linked to the scatter value 430 (i.e. whether the procedure data set 450 has the scatter value 430).

In step 307a, the test instance 2 is set up to determine during the test in step 307b that the scatter value 430 of the personal data set 320 is valid and that the procedure identification number of the procedure data set 450 is valid and that the procedure data set 450 has the scatter value 430 (yes case). The test instance 2 is then set up to indicate in step 308c that the test result is validated (validation result of the procedure result is positive). The procedure data set 450 can also be displayed, preferably a time stamp (time and date) of the antigen test for SARS-CoV-2 or a status of the antigen test for SARS-CoV-2 is also displayed as procedure information, more preferably the test result of the antigen test for SARS-CoV-2 according to background system 40.

FIG. 9a shows a structure of an anonymized personal data set in the background system 40. The anonymized personal data set 410 in the background system 40 comprises a scatter value 430 of the personal data set 320. In addition to the scatter value 430, the anonymized personal data set 410 (=“Remote_Testee”) can also comprise a log entry with which procedure events, in particular failed object identifications, can be stored with a time stamp.

The anonymized personal data set 410 (=“Remote_Testee”) can also include a personal identification number with which the person 1 can be uniquely referenced on the background system 40.

FIG. 9b shows a structure of a personal data set 320 of the storage unit 32 of the terminal 30. The personal data set 320 comprises biometric data. Biometric data are in particular facial geometry data and/or fingerprint data. The personal data set 320 also comprises personal data. Personal data includes, in particular, first name, surname, date of birth, address and document number. The personal data set 320 also includes a quarantine end date field in which the end of a quarantine period (for SARS-CoV-2=2 weeks) can be entered.

In FIG. 9c, a structure of an object identification data set 420 in the background system 40 is shown. The object identification data set 420 may, for example, comprise an object type. This is useful if a procedure can be performed with different types of objects 20. The object type can then be used to customize the guidance and documentation accordingly. The object identification data set 420 can, for example, comprise a manufacturer specification, a production date, an expiry date, an object property and/or a certificate. This allows the object 20 to be best identified in the method 200 and useful information for performing the procedure to be provided.

In FIG. 9d, a structure of a procedure identification data set 321 of the storage unit 32 of the terminal device 30 is shown. The procedure identification data set 321 of FIG. 9d comprises a procedure identification number, which is preferably unique throughout the procedure and system. The procedure identification record 321 further comprises the procedure documentation, which comprises results and/or intermediate results and/or snapshots (screenshots) of the execution.

FIG. 9e shows a structure of a procedure data set 450 in the background system 40. The procedure data set 450 may, for example, comprise a procedure serial number.

The procedure data set 450 may, for example, comprise a status of the object 20. A status of the object 20 may be “initialized”, for example if the object 20 exists in the procedure 200 but has not yet been registered in the background system 40. A status of the object 20 may be “established”, for example if the object 20 exists in the method 200 and is also registered in the background system 40. A status of the object 20 may be “in use”, for example if the object was captured in the method 200 in step 202 and is then used. A status of the object 20 may be “completed”, for example when the object 20 has been used in the method 200 and has generated a test result.

The procedure data set 450 can comprise a procedure result. The procedure result can be “positive”, “negative” or “invalid”. An invalid procedure result is output if the procedure was used incorrectly or person 1 and/or object 20 could not be identified/traced in accordance with the procedure. A positive procedure result can be issued if a procedure has been completed without errors or manipulation. Based on a virus detection test as a procedure, a positive test result is the detection of viruses by the test cassette. Based on a virus detection test as a procedure, a negative test result is the lack of detection of viruses by the test cassette.

The procedure data set 450 may comprise a timestamp, for example a time or date. The procedure record 450 may comprise the scatter value 430. The procedure record 450 may comprise a procedure identification number. The procedure record 450 may comprise a person identification number.

FIG. 10a shows a first embodiment for preparing a test according to step 2044a. In step 2044a, a request to unpack the antigen test for SARS-CoV-2 is displayed, whereby the test cassette 20 and also all aids 50 must be placed in the field of view (detectable area or predefined area) of the camera 31a of the terminal device 30. In addition, in step 2044a, the person is instructed to place the QR code 210 (=machine-readable object identification data set 21) of the test cassette 20 in such a way that the camera 31a of the terminal device 30 can capture this QR code 210 (step 2021 in FIG. 3).

In this embodiment according to FIG. 10a, the QR code 210 has already been applied to the test cassette 20 during the production of the test. The QR code 210 can be printed or applied using an adhesive. The person has been able to purchase the test with the information that the test is already fully set up for validation (according to procedure 300) and that no additional QR code 210 needs to be purchased.

FIG. 10b shows a second embodiment for preparing a test according to step 2044a. In step 20441, (firstly) instructions for capturing the test package (in the unpacked state) are displayed and (secondly) a request to unpack the antigen test for SARS-CoV-2 is displayed. By capturing the test package, the method 200/system 2000 is adapted to capture the information about the test type (object type in the object identification data set 420).

Then, in step 20442 (firstly), a prompt is displayed to apply a QR code 210 to the test cassette 20 to validate the antigen test for SARS-CoV-2. The person must then apply the QR code 210 to the test cassette 20. It can be applied using an adhesive. For example, the QR code 210 is designed as an adhesive patch. An adhesive layer on the underside of the QR code patch preferably ensures a mechanically non-detachable connection between the QR code patch and the test cassette 20. Then, in step 20442 (secondly), the person 1 is instructed to place the applied QR code 210 (=machine-readable object identification data set 21) on the test cassette 20 in such a way that the camera 31a of the terminal device 30 can detect this QR code 210 (step 2021 in FIG. 3).

In this embodiment according to FIG. 10b, the QR code 210 has not been applied to the test cassette 20 during production of the test. The person has been able to purchase the test with the information that the test is not set up for validation (according to procedure 300) and does not contain a QR code 210. If person 1 wishes to have their test result validated (in accordance with procedure 300), person 1 must manually apply a QR code 210. The person 1 can acquire the QR code 210 in parallel with the test itself. In this embodiment according to FIG. 10b, the QR code 210 is only applied to the test cassette 20 by the person 1 before the test is carried out.

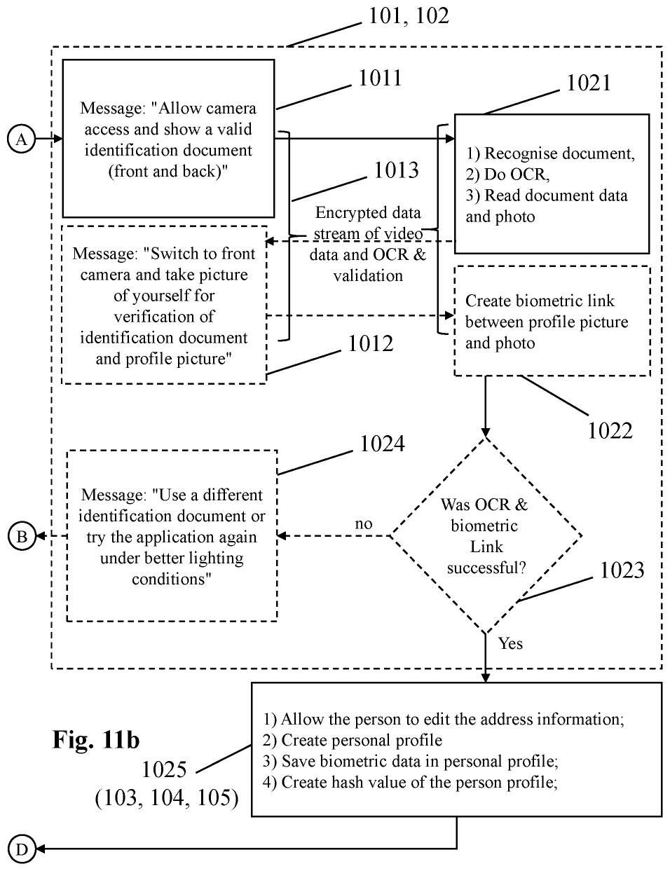

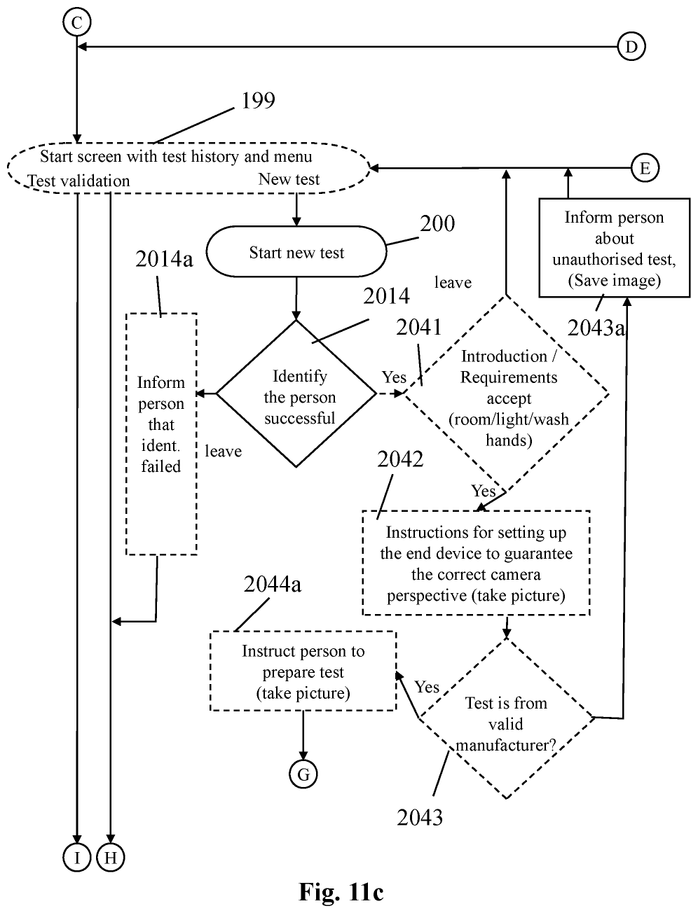

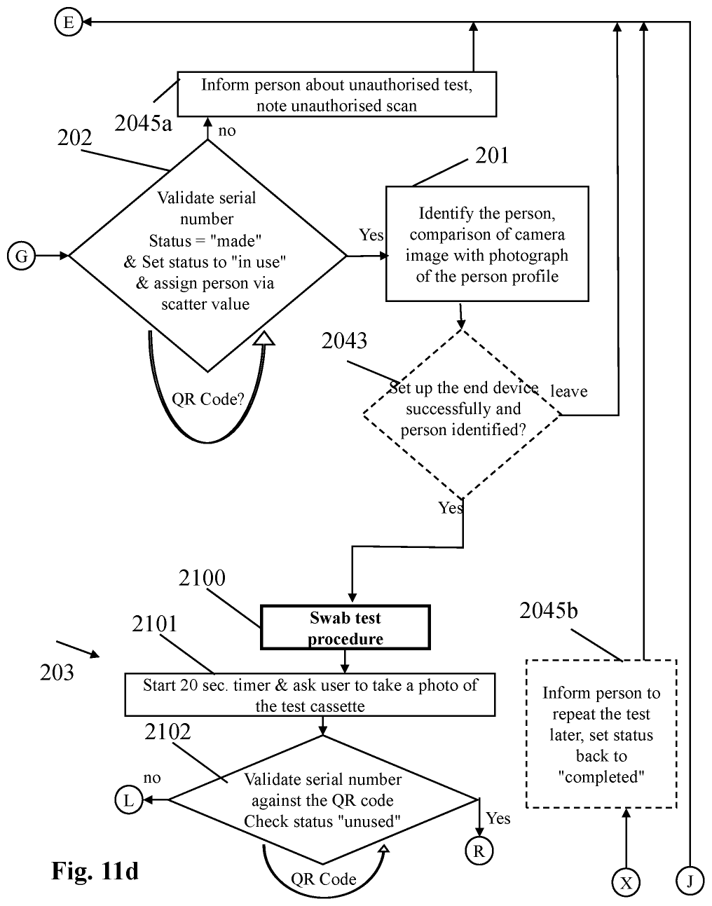

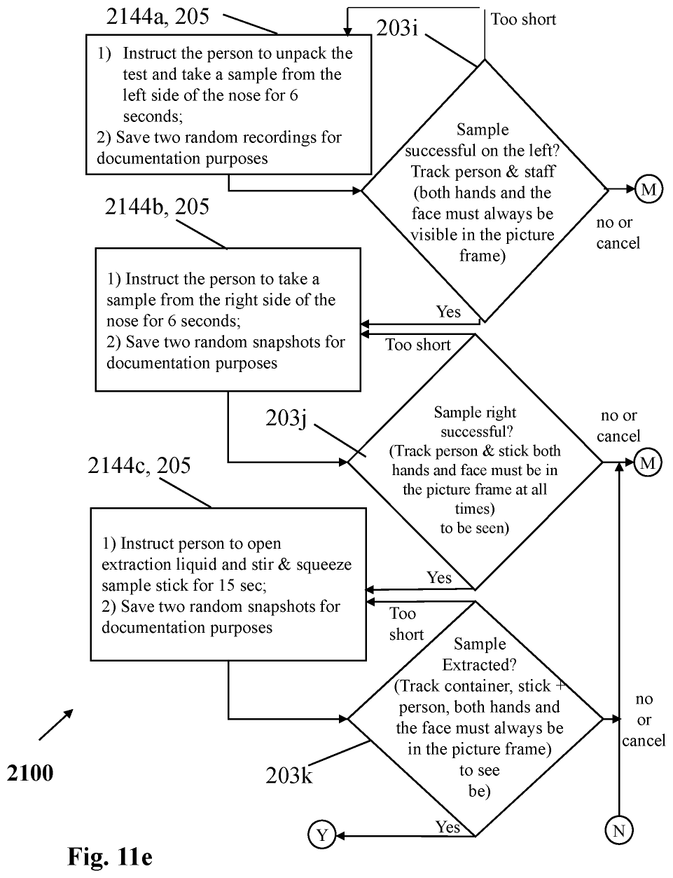

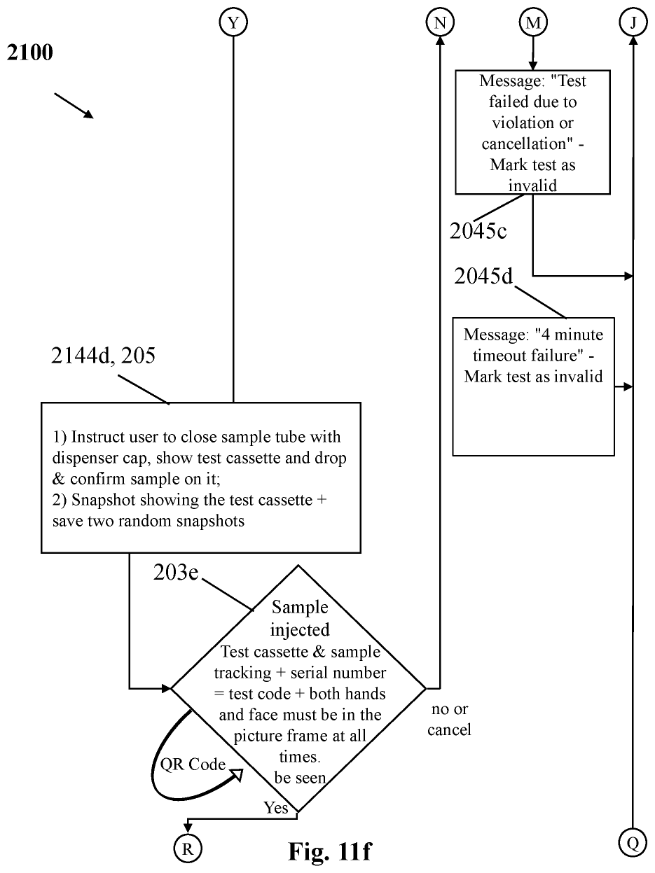

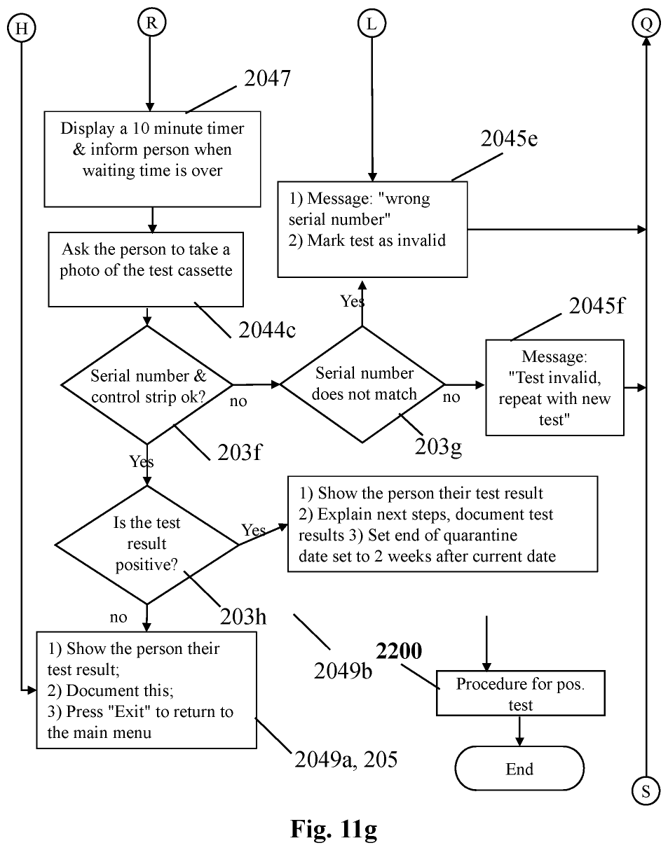

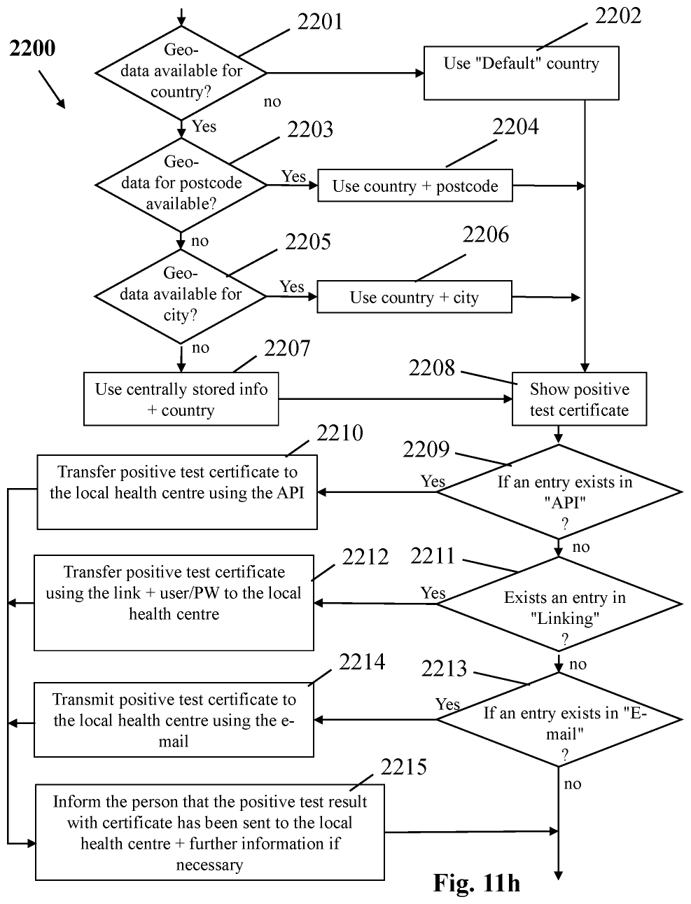

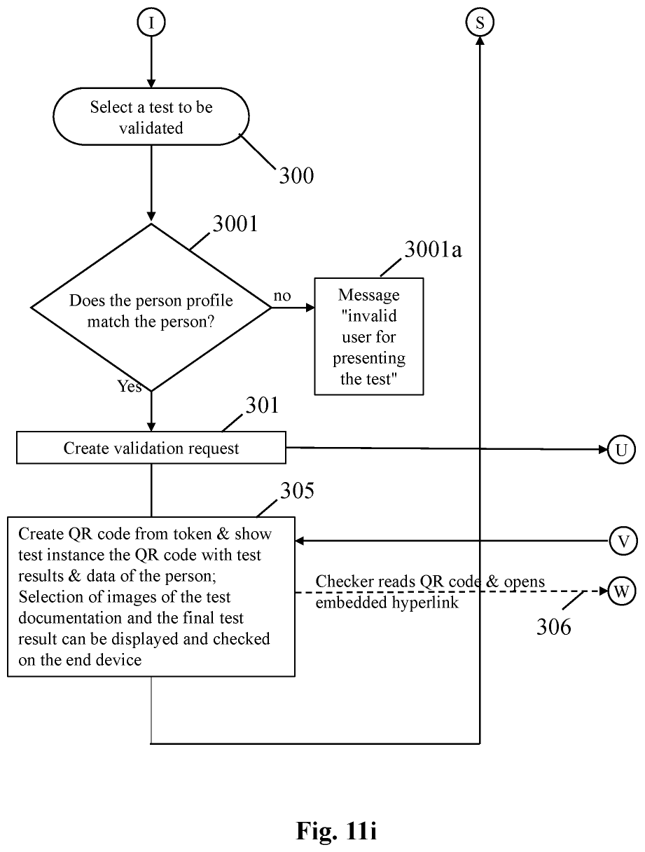

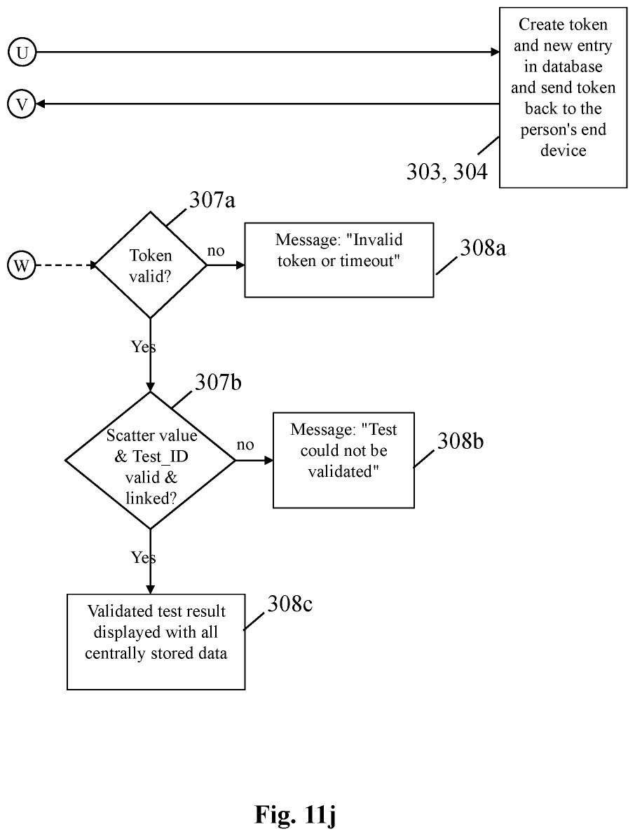

FIG. 11a-j shows a further flowchart of the methods 100, 200, 300 for a test, in this case an antigen test for SARS-CoV-2, as an exemplary procedure. This flowchart enables the test to be used as a swab test.

A method is proposed whose test can be easily identified by the terminal device 30 using a machine-readable code, for example by a QR code or a tag such as an NFC tag, RFID tag, Bluetooth tag, etc. on the test, for example the test cassette 20. The machine-readable code can, for example, be applied during production of the test and then entered into the background system 40 by machine reading.

Alternatively, according to the invention, it is also envisaged that a test without machine-readable code (so-called system-neutral test) is acquired. In addition, a machine-readable code is acquired in order to be able to participate in the method/system according to the invention. During the preparation of the test, for example in preparation of step 202 (FIG. 11d), the machine-readable code is applied to the test (the test cassette 20). For this purpose, for example, a QR code is placed as a sticker on the test cassette 20 as a machine-readable code by an adhesive layer.

During the procedure, in particular in steps 202, 2102 and 203e, the presence of the machine-readable code is queried. If the machine-readable code cannot be read by the terminal 30 in these steps, the person 1 is prompted to correct the position of the test (e.g. move closer, etc.). If a correction is not made in time, the test is cancelled and the test is discarded as “invalid” and the serial number of the test is marked as “used/used” in the background system.

Another general difference to the process sequence of FIGS. 8a-k is the documentation by screenshots instead of a complete video sequence recording. It has been found that a targeted recording of images randomly during a process step to be documented, in particular during steps 2042, 2043a, 2044a, 2144a-d, 2044c, is more memory-efficient but still ensures process reliability. The recorded images are stored with a time stamp in a data memory locally in the terminal device 30 and are protected against unauthorized access. A user login is required to access the images.

In addition to the AI-monitored test procedure, the storage of the images also provides documentation, i.e. a second level of monitoring.

In a minimum version, only eight images per test procedure are captured and stored. The images are given a time stamp for documentation purposes when they are stored.

These images form proof of documentation, for example for submission to an employer, to ensure that the test has been carried out correctly.

The flow chart of FIGS. 11a-11j corresponds in large parts to the flow chart of FIGS. 8a-k and statements relating to FIGS. 8a-k also apply initially to FIGS. 11a-j, unless it is described below as a difference to FIGS. 8a-k.

The flow diagram in FIG. 11a is identical to FIG. 8a and reference is made to the description in FIG. 8a.

The flow diagram in FIG. 11b is identical to FIG. 8b and reference is made to the description in FIG. 8b.

With reference to FIG. 11c, if the person 1 chooses to perform a new antigen test for SARS-CoV-2 in step 199, the method 200 is carried out according to FIGS. 3 and 4. In step 2014, the terminal device 30 checks whether the identification of person 1 was successful. For this purpose, the current camera image of the terminal device 1 can optionally be captured again (i.e. the face 1a of the person 1) and compared with the biometric data of the personal data set 320 (see also comments on FIG. 3).

If it is determined in step 2014 that the identification of person 1 was not successful (abandonment case), the person is (optionally) informed in step 2014a that the identification was not successful and the common selection screen is then shown again in step 199 (FIG. 11c).

If the terminal device 30 determines in step 2014 that the identification of the person 1 was successful (yes case), an introduction screen is displayed on the display unit 34 of the terminal device 30 in the optional step 2041 and it is checked by the evaluation unit 33 of the terminal device 30 whether the person 1 has given the environmental conditions (light, room) and his willingness to perform the procedure 200.

If, in step 2041, the terminal device 30 determines that person 1 has not submitted readiness, the procedure is terminated via an “Exit” button.

If, in step 2041 (FIG. 11c), the terminal device 30 determines that the person 1 has given readiness (yes case), in the optional subsequent step 2042, the person 1 is shown instructions for setting up the terminal device 30 on the display unit 34 of the terminal device 30. With this instruction, the person 1 is instructed how to unpack the test and place all elements of the test in front of him, and to activate the rear camera 31b of the terminal device 30 to take a picture of the unpacked test before unpacking the test. The image is saved for documentation purposes. In the following step 2043, it is checked whether the test is from a valid manufacturer (i.e. a manufacturer authorized or certified or registered for this procedure).

If, in the optional step 2043, the terminal device 30 determines that the test is not from a valid manufacturer, the person is informed of this in step 2043a and the common selection screen is then displayed again in step 199 (FIG. 11c).

If, in the optional step 2043, the terminal device 30 determines that the test is from a valid manufacturer, the person 1 is instructed in step 2044a to unpack and prepare the test and to hold a machine-readable code, e.g. QR code, in the rear camera 31b of the terminal device 30. The terminal device 30 takes a picture when the machine-readable code is recognized, also for documentation purposes.

In the subsequent step 202 (FIG. 11d), an attempt is made to identify the test, for example the test cassette 20 (the test object). The QR code 210 of the test 20 is captured and it is checked whether a procedure serial number encoded in the QR code 210 matches a procedure serial number of a procedure data set 450 of the background system 40. In addition, the step 202 can check whether a status of the procedure record 450 is set to “manufactured” and whether this status can be set to the status “in use”. In addition, the step 202 can check whether the scatter value 430 of the person data set 320 of the procedure data set 450 can be assigned to the person 1.

If the terminal device 30 determines in step 202 that the identification of the test 20 was not successful (no case), the person is informed in the subsequent step 2045a by a message on the display unit 34 of the terminal device 30 that the test 20 was not recognized or that the present test 20 has an invalid/invalid QR code. The selection screen in step 199 is then shown (FIG. 11c).

If the terminal device 30 determines in step 202 that the identification of the test 20 was successful (yes case), the person 1 is identified in step 201. For identification 201, captured biometric data is compared with the biometric data of the personal data set 320 by the identification unit of the terminal device 30. This biometric data of the personal data set 320 can be retrieved from the storage unit 32 of the terminal device 30. This biometric data of the personal data set 320 was preferably obtained using the method 100 of FIG. 1 (system 1000 of FIG. 2). This may require decryption of the personal data set 320 (if encryption was performed in step 104 of the method 100).

The optional step 2043 checks whether test 20 and person 1 have been successfully identified. If the answer is no, person 1 (optional and not shown in FIG. 11d) is informed in step 2014a that the identification of test 20 and person 1 was not successful and the joint selection screen is then shown again in step 199 (FIG. 11c).

In the Yes case of step 2043, the test procedure can be performed because both the person 1 and the test object 20 have been identified (steps 201, 202). This means that the test procedure can now be followed up in accordance with step 203, in particular procedure 2100.

Step 2101 starts a timer and prompts the user to take a picture of the test 20, for example the test cassette. In the subsequent step 2102, the scan of the machine-readable code made in step 2101 is compared with the serial number and the status of the test is checked for “unused”. If this check is successful (yes case) in step 2102, the process is continued as shown in FIG. 11g.

FIGS. 11e and 11f now describe the tracing of the swab test (=swab test) according to step 2100. FIGS. 11e and 11f essentially correspond to FIGS. 8h and 8i and only differences are pointed out here, for example in step 203e of FIG. 11e the machine-readable code of the test 20 is additionally verified.

FIG. 11g essentially corresponds to FIG. 8g and only differences are pointed out, for example step 2045e of FIG. 11g is achieved both starting from the yes case of step 203g and from the no case of step 2102.

In FIG. 11g, between steps 2049b and the end of the procedure (cf. FIG. 8g), a further procedure 2200 is now proposed, which records the reporting of a positive test result, as described in FIG. 11h.

According to this procedure 2200, a centrally stored table that is cryptographically protected against unauthorized reading is used to check where the positive test result should be automatically reported after the end of the test. The table can contain data such as country, postcode, city or API access keys, email addresses and user+password information for the local health center or person 1. This data can be queried when a user profile is created (FIGS. 11a & 11b) or data on local health centers is automatically stored centrally.

The background system 40 maintains a central database with rules for further action in the event of positive test results, which may vary from municipality to municipality.

The terminal device 1 can now provide geodata from its current position to the application, for example GPS location data.

Step 2201 queries whether the geodata country information provided is also contained in the central table. If the answer to step 2201 is no, the country of the central table is defined by default in step 2202.

If step 2201 is Yes, step 2203 queries whether the geodata postcode information provided is also contained in the central table. If the answer to step 2203 is yes, the stored country+postcode is used in step 2204.

If the answer to step 2203 is no, step 2205 queries whether the geodata city information provided is also contained in the central table. If the answer to step 2205 is yes, the stored country+city are used in step 2206.

In the No case of step 2205, the stored information is used in step 2207 in conjunction with the matching country in step 2207.

The positive test certificate is displayed in step 2208 following step 2022 or 2207.

Step 2209 then queries whether an API entry relating to the local health center is stored in the centrally stored table. If the answer in step 2209 is yes, the positive test certificate is transferred to the local health center in step 2210 using the API and person 1 is informed of the dispatch in step 2215.

If the answer to step 2209 is no, the next step 2211 asks whether an internet link to the local health center is stored in the centrally stored table. If the answer in step 2211 is yes, the positive test certificate is transmitted to the local health center in step 2212 using the internet link with user and password and person 1 is informed of the dispatch in step 2215.

If the answer to step 2211 is no, the next step 2213 asks whether an email entry relating to the local health center is stored in the centrally stored table. If the answer in step 2213 is yes, the positive test certificate is sent to the local health center in step 2214 using the stored email address and person 1 is informed of the dispatch in step 2215.

The application is then ended (FIG. 11g).

If a validation of an already performed test is selected in the common selection screen according to step 199 of FIG. 8c, the procedure according to FIG. 11i and FIG. 11j is run through. The flow chart in FIG. 11i is identical to FIG. 8j and reference is made to the description of FIG. 8j. The flow chart in FIG. 11j is identical to FIG. 8k and reference is made to the description in FIG. 8k.

The recordings obtained during the test procedure are stored in an object memory locally on the terminal device 30 and can only be retrieved via the test procedure application on the terminal device 30. This ensures a high level of data security.

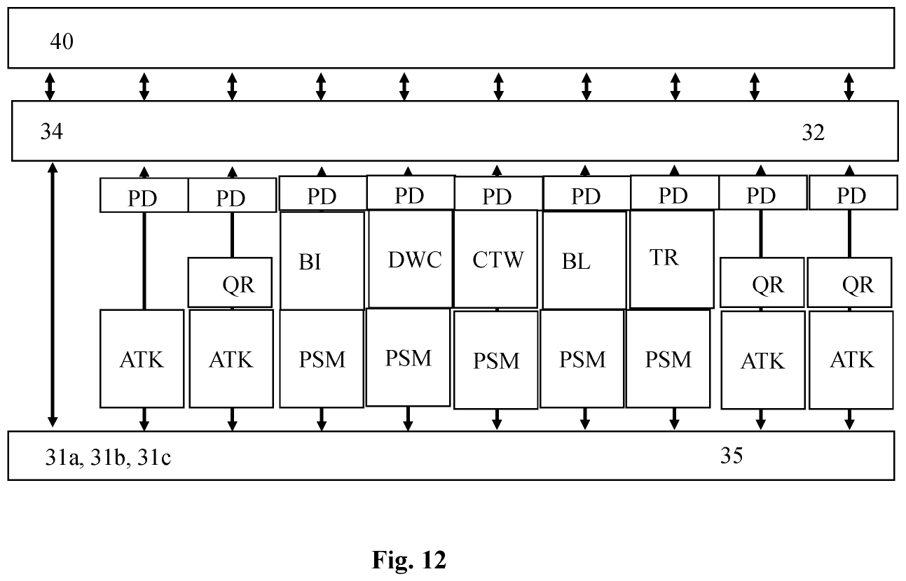

FIG. 12 shows a modular architecture of the test procedure application on the terminal 30, which is described below.

During process execution, user 1 is guided through the test procedure, whereby the user is monitored during the process to ensure that all steps are carried out correctly and also to document the execution of the process. In addition, the process flow is automatically controlled by the activities of user 1.

In order to be able to orchestrate these four functionalities (control, monitoring, documentation, management) in a test procedure application of a mobile terminal device, a test procedure application architecture was developed that allows individual process steps to be managed and interpreted on the basis of available technology modules that are plugged together on a video stream (similar to Lego bricks).

Individual technology modules allow partial functionality, but only the orchestration of all modules together guarantees the continuous process flow using the video stream to control, monitor, document and manage the application flow.

Some of these building blocks may themselves be composed of sub-building blocks. The underlying video stream can alternate between front and rear cameras 31a, 31b of the terminal device 30 and between fixed observation positions, with the terminal device 30 being positioned on a stand, for example, to (1) ensure a calibrated position with always the same angle and (2) enable user-based tracking of specific components/tools used in the process.

The architecture enables a new type of application for mobile device 30, where various video and AI functionalities are combined to manage more complex tasks or processes, such as controlling, monitoring, documenting and/or managing a test procedure through the test procedure application of mobile device 30.

Thanks to the developed application architecture, all steps can now run in parallel with process execution and the disclosure of personal data is avoided.

FIG. 12 shows the following modules that are used in the modular architecture:

Module “BI”: Biometric identification—based on an AI model, the video image of user 1 is compared with a stored biometric profile to identify user 1, in particular in the steps.

Building block “ATK”: Authorize Test Kit—an AI model that is used to identify the test kit that the user shows to the app. This model is supplemented by OCR technologies.

QR” module: QRE code reader: The QR code on the test plays an important role in monitoring the test cassette in the process. To ensure that the cassette remains the same throughout the test, the QR code is printed or attached, for example via an adhesive layer that cannot/may not be removed without destroying the QR code. This QR code gives the test cassette a serial number and enables the test procedure application to update the status of the test, the test cassette in the background system 40, so that a cassette once used in a test cannot be used again later.

Module “TR”: Test result: The TR module, for example Covid-TR, is an AI model that interprets the result of the test cassette. It returns the status of the test cassette as a result, in particular unused test cassette, cassette in process, invalid test, positive test, negative test.

Block; “DWC”: A block to determine the position and orientation of the test window of a test cassette in the video image and to cut out the section of the test window.

CTW” module: A module for interpreting the test result on the basis of the cut-out image, in particular unused test cassette, cassette in process, invalid test, positive test, negative test.

Block “BL”: A block to interpret whether the test is in use. During a test, a red film can rise in the test window. The comparison of 2 images with a time difference to interpret whether the film is growing provides the result “Cassette in process” from the BL function block.

Building block “TKO”: Test kit object is an AI model to identify and track components of the test in the video image

Building block “PSM”: A pose detection AI model to interpret the pose of the user in the image. The PSM model is used to ensure that the cameras 31a, 31b are positioned at the correct distance from user 1 in order to capture the test area and user 1 in the video image.

PD” module: A module for photo documentation takes pictures in critical parts of the test procedure and documents the test process with a time-stamped image that is stored in encrypted form in the app for test documentation.

Module “DM”; A basic module that displays the image of the respective camera 31a, 31b to the user.

The AI models run partly in the test procedure application of the terminal device 30 and partly as a web service function on a server of the background system. In the case of the central AI functions, this is implemented as a web service in order to collect additional training data.

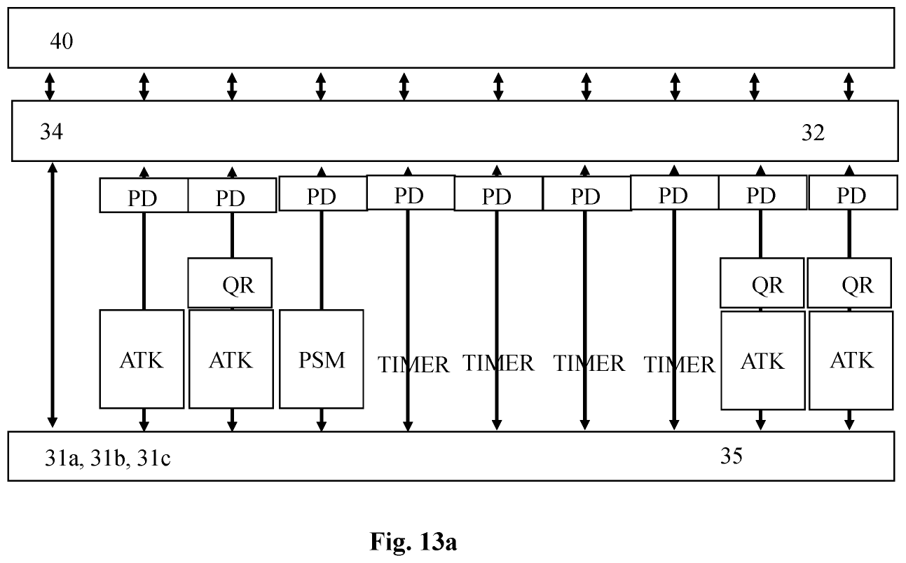

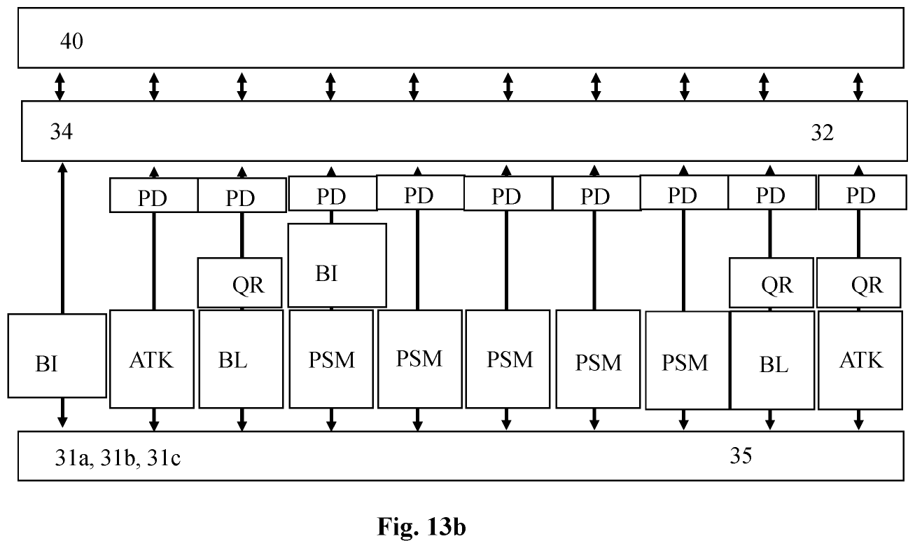

FIG. 13a to 13c show different versions of the test procedure application architecture.

For implementation and to ensure user-friendliness, the app architecture is staggered over several versions. In earlier versions, the AI models are replaced by manual steps by the user, reusing the functionality of the app but simplifying it for the user.

FIG. 13a shows a version 1 of the test procedure application architecture. This uses a fully guided test process with manual start of the timer (four times in total). The timer is used for the two sample collection screens of steps 2144a, 2144b, the soaking of the swab in step 2144c, the dripping of the test liquid into the test cassette in step 2144d. In addition, this version 1 includes the identification of the test according to step 202 and of the user according to step 201 as well as the creation of the photo documentation (at least 8 images) and the reading and monitoring of the test cassette during steps 2047 and 203f.

FIG. 13b shows a version 2 of the test procedure application architecture. Version 2 is based on version 1 and also includes (1) a biometric check of the person so that no second screen is necessary, (2) a gesture recognition for recognizing the sampling and automatic timer start as soon as the swab is in the nose in steps 2144a, 2144b; (3) a gesture recognition for recognizing the washing of the swab and automatic timer start once the swab is in the tube in step 2144c; and (4) a gesture recognition for dripping the liquid into the test cassette according to step 2144d.

FIG. 13c shows a version 3 of the test procedure application architecture. Version 3 is based on version 3 and also includes gesture tracking so that the hands and body are always in the image.

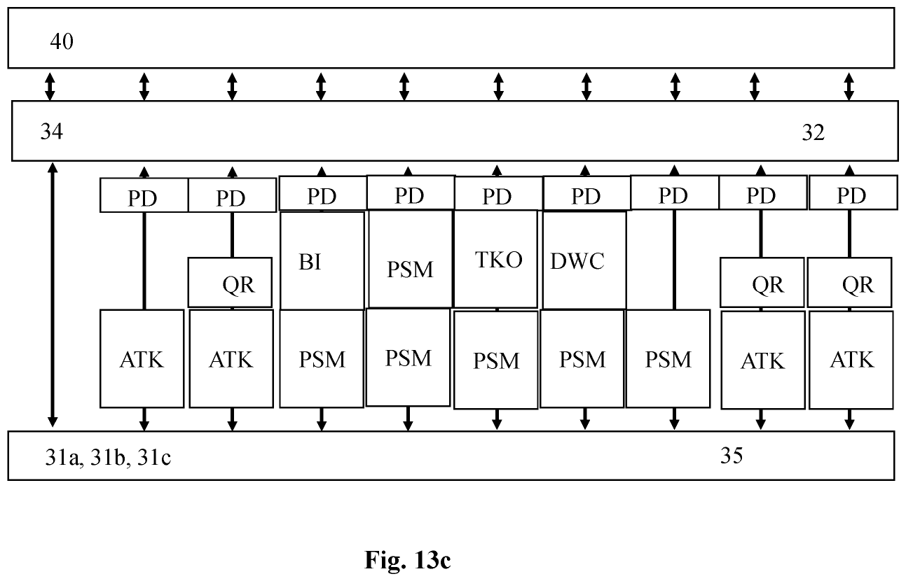

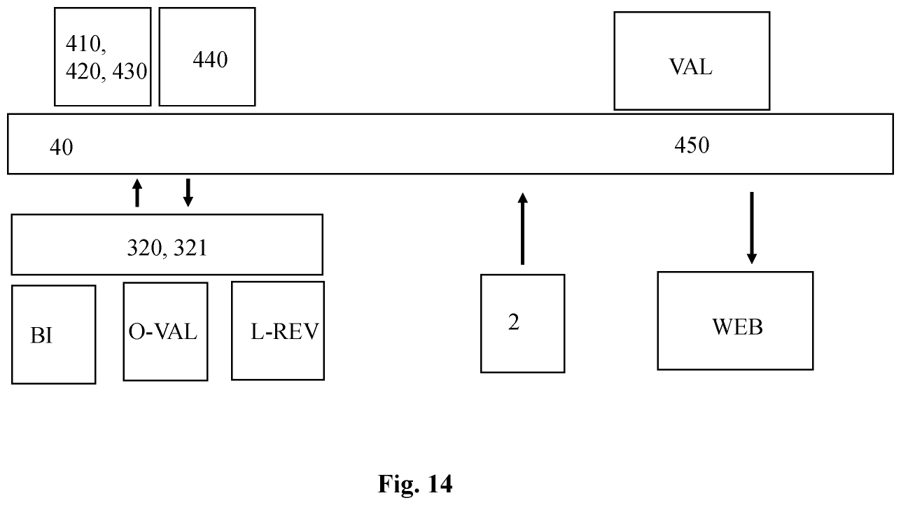

FIG. 14 shows a validation procedure for test certificate VAL. At the end of each process execution, a certificate VAL is issued for the successful execution of the process, for example a COVID-19 test, the process result (test result) and documentation of the process as part of the certificate VAL.

The criteria for this certificate VAL are that it is only issued at the request of user 1, that it is verifiable by third parties (auditor 2), that it is machine-readable and exportable if required by local legislation or if this is the wish of user 1.

The data for this certificate is distributed between the terminal device 30 and the background system 40 for data protection reasons. The certificates VAL are not stored, neither in the test procedure application of the terminal device 30 nor in the background system 40, in order to ensure that they cannot be manipulated and to prevent them from being made accessible to unauthorized third parties.

As soon as user 1 requests a specific certificate, he must first identify himself via the biometric identification BI (step 201). The application then requests this specific certificate via a validation window O-VAL in the background system 40 and in return receives the centrally stored data (410, 420, 430) for the certificate VAL and a validation token 440, which is converted into a machine-readable QR code.

Together with the locally stored data, 320, 321, the certificate VAL is created and displayed on the terminal device 30 of user 1 together with the photo documentation of the process. The user 1 can now present the certificate VAL to a human or machine verifier 2, at which point the identity of the user 1 is already verified.

The auditor 2 can simply check the certificate VAL and the documentation library visually and/or validate it by scanning the validation token 440 either with the auditor's mobile device 2 or with a QR code reader. The validation token 440 contains a link to a web service WEB with the validation request for the submitted certificate VAL.

Each certificate has a validity period of 10 minutes, for example, after which user 1 must use a new one to identify himself. The web service WEB returns whether the certificate VAL is still valid. If the test results are validated, all test-related data is also returned.

In the case of a machine tester, system 40 can anonymously check whether the person 1 presenting the certificate VAL (who is then already identified as the person 1 who performed the test) has a valid test result to grant access.

The following is a list of examples:

Example 1: A system (2000) for computer-aided tracking of a test procedure to be performed by a person (1), in particular for a virus detection test, preferably for performing a SARS-CoV-2 test, wherein the system (2000) comprises:
an identification unit of a terminal (30) adapted to identify (201) the person (1) immediately before the person (1) performs the test procedure;
wherein the identification unit of the terminal device (30) is further adapted to identify (202) a test object (20), preferably a test cassette, in particular for a SARS-CoV-2 test, more preferably a test strip, more preferably a reagent in a container, more preferably a test sensor, immediately prior to performing the test procedure, comprising the identification unit:
a camera (31) of the terminal (30), adapted to capture (2021) an object identification data set (21) as a machine-readable data set; and
an evaluation unit (33) of the terminal device (30), set up for comparing (2022) the captured object identification data set (21) with an object data set (420) of a background system (40);
a tracking unit of the terminal device (30), which is adapted to track (203) one or more positions of the person (1) during the performance of the test procedure by methods of computer-aided gesture and/or pattern and/or image recognition by checking whether both hands (12) of the person (1) are in a predefined area or an area detectable by the camera (31a) of the terminal device (30) during the entire performance of the test procedure;
the tracking unit of the terminal device (30), further adapted to track (203) one or more positions of the test object (20) from the shape of the object during the execution of the test procedure by computer-aided gesture and/or pattern and/or image recognition methods; and
a display unit (34) of the terminal device, adapted to instruct (204) the person (1) to perform the test procedure during the execution of the test procedure.

Example 2: The system (200) according to example 1, wherein the checking by the tracking unit of the terminal device (30) also comprises checking whether the face (1a) of the person (1) is in the predefined area or the area detectable by the camera (31a) of the terminal device (30) during the entire execution of the test procedure.

Example 3: The system (200) according to Example 2, wherein checking by the tracking unit of the terminal device (30) also comprises checking whether the face (1a) and both hands (1b) of the person (1) are in the predefined area or the area detectable by the camera (31a) of the terminal device (30) during a set-up of a test set for the test procedure.

Example 4. The system (200) according to example 2 or 3, wherein the checking by the tracking unit of the terminal device (30) also comprises:
whether the face (1a) and the two hands (1b) of the person (1) is in the predefined area or the area detectable by the camera (31a) of the terminal device (30) during a delivery of a saliva sample; and/or
whether the face (1a) and both hands (1b) of the person (1) are in the predefined area or the area detectable by the camera (31a) of the terminal device (30) during an addition of an extraction liquid into the saliva sample; and/or
whether the face (1a) and the two hands (1b) of the person (1) are in the predefined area or the area detectable by the camera (31a) of the terminal device (30) during shaking of the saliva sample for a predetermined period of time; and/or
whether the face (1a) and the two hands (1b) of the person (1) and the object (20) are in the predefined area or the area detectable by the camera (31a) of the terminal device (30) during an injection of the saliva sample onto the object (20).

Example 5: The system (200) according to any of the preceding examples, wherein the tracking unit of the terminal device (30) is further adapted to recognize one or more movements of the person and/or the test object based on the shape of the test object (20) during the execution of the test procedure by methods of computer-aided gesture and/or pattern and/or image recognition.

Example 6: The system (200) according to example 5, wherein the tracking unit of the terminal device (30) checks whether the test object (20) is within a predefined range during the entire execution of the test procedure.

Example 7: The system (200) according to any of the preceding examples, wherein the identification unit comprises for identifying (202) the test object:
the camera (31) of the terminal device (30), set up to capture (2011) a face (1a) of the person (1);
an evaluation unit (33) of the terminal device (30), set up for evaluating the captured face (1a) by methods of computer-aided face and/or pattern and/or image recognition for obtaining biometric data and for comparing (2012) the obtained biometric data with biometric data of a personal data set (320), wherein the personal data set (320) is stored in a storage unit (32) of the terminal device (30).

Example 8: The system (2000) according to example 7, wherein the identification unit of the terminal device (30) is further adapted to repeat the identification (202) of the person (1) at one or more random times during the execution of the test procedure.

Example 9: The system (2000) according to example 7 or 8, wherein the terminal device (2) displays an error message on a display unit (35) of the terminal device (2) if the evaluation unit (33) of the terminal device (30) determines during comparison (2012) that the captured biometric data does not match the biometric data of the personal data set (320).

Example 10: The system (2000) according to any of the preceding examples, wherein the identification unit of the terminal device (30) is further adapted to identify, immediately before the person (1) performs the test procedure, all aids (50) to be used during the test procedure by the terminal device (30) on the basis of their shape by methods of computer-aided face and/or pattern and/or image recognition.

Example 11: The system (2000) according to example 10, wherein the aid (50) is: a swab for taking a nasal or saliva sample from the person (1); and/or a liquid container.

Example 12. The system (2000) according to example 10 or 11, wherein each aid (50) used is detected by the camera (31) of the terminal device (30) and the detected aid (50) is analyzed by methods of computer-aided face and/or pattern and/or image recognition by an evaluation unit (33) of the terminal device (30) in order to identify the aid (50).

Example 13. The system (2000) according to one of the preceding examples, wherein the machine-readable data set (210, 220) of the object (40) is validatable by querying (2023) a corresponding object data set (420) in a background system (40), wherein after the validation of the machine-readable data set (210, 220) the object data set (420) in the database (41) of the background system (40) is invalid (2024).

Example 14. The system (2000) according to one of the preceding examples, wherein the tracking unit of the terminal device (30) is further adapted to detect (2031) an object property of the object (20) by the camera (31) of the terminal device (30), wherein the detected object property of the object (20) can be analyzed (2032) by methods of computer-aided face and/or pattern and/or image recognition by an evaluation unit (33) of the terminal device (30) and the analyzed object property of the object (20) is compared with an object property to be expected during the test procedure.

Example 15: The system (2000) according to example 14, wherein an object property is the application of a swab, saliva or spit sample of the person (1) to the test cassette of an antigen test for SARS-CoV-2.

Example 16: The system (2000) according to example 14 or 15, wherein an object property is the discoloration of a test strip of the test cassette of a SARS-CoV-2 test.

Example 17: The system (2000) according to examples 14 to 16, wherein an object property is the appearance of one or more test strips on the test cassette of a SARS-CoV-2 test.

Example 18. The system (2000) according to one of the preceding examples, wherein the terminal device (2) displays an error message on a display unit (35) of the terminal device (2) if the evaluation unit (33) of the terminal device (30) determines during analyzing (2032) that the detected position of the person (1) and/or the detected movement of the person (1) and/or the detected position of the object (1) and/or the detected movement of the object (1) is outside the predefined range or the range that can be detected by the camera (31a) of the terminal device (30).

Example 19: The system (2000) according to one of the preceding examples, wherein for documenting the execution of the test procedure, the execution of the test procedure is captured and stored at one or more random points in time during the execution of the test procedure by the camera (31) of the terminal device (30).

Example 20: The system (2000) according to any of the preceding examples, wherein instructional information is displayed on the display (34) of the terminal (30), wherein an instructional information is displayed:
depending on the execution of the test procedure and/or
depending on the tracking (203) of positions and movements of the person (1) and the object (20) by the terminal device (30) during the execution of the test procedure;
after a waiting time specified in the test procedure has elapsed.

Example 21: A system (3000) for computer-aided validation of a procedure performed by a person (1), the system (3000) comprising:
an input unit (35) of a terminal device (30) adapted to receive a person's input for requesting (301) a validation of a procedure result;
a control unit (33) adapted to:
generation of a scatter value via a personal data set stored in a storage unit (32) of the terminal device (30) of the terminal device (320)
generating a validation request comprising the scatter value and a procedure identification record procedure identification record (321) of the procedure result to be validated; and procedure result to be validated;
sending the validation request to a background system (40);
receiving (304) in the terminal device (30) a validation token from the background system (40); and background system (40);
a display unit (34) of the terminal device, adapted to display (305) a machine-readable code with the validation token (440) and the procedure result;
an inspection body, set up for the:
checking (306) the machine-readable code while checking the validation token;
displaying (307) a validation result with respect to the procedure result together with the procedure result and the scatter value over the personal data set and preferably procedure information.

Example 22: The system (3000) according to example 21, where the validation token defines a validity period for the procedure result.

Example 23: The system (3000) according to any of the preceding examples 21 or 22, wherein the procedure information is a procedure date and/or a procedure time according to the background system (40).

Example 24: The system (3000) according to any one of the preceding examples 21 to 23, wherein the procedure identification record has been generated by the terminal device.

Example 25. The system (3000) according to any of the preceding claims 21 to 24, wherein the machine-readable code is, preferably, a QR code and wherein the verifier (2) comprises a capture unit to read the machine-readable code.

Example 26: The system (3000) according to any one of the preceding examples 21 to 25, wherein the procedure result is queried (308) by the background system.

Example 27: A system (1000) for setting up a personal profile comprising a terminal device (30) and an identification document (10), the system (1000) comprising:
a capture unit (31) of the terminal device (30), set up for capturing (101) data (11, 14, 15) of an identification document (10) of the person (1);
an evaluation unit (33) of the terminal device (30), set up for:
analyzing (102) the data (11, 14, 15) of the captured identification document (10) to obtain a personal data set (320) comprising biometric data and personal data of the person (1);
a storage unit (32) of the terminal device (30), set up for storing (103) the personal data set (320) obtained as a personal profile.

Example 28: The system (1000) according to example 27, wherein the evaluation unit (31) of the terminal device (30) is further configured to generate (105) a scatter value (430) of the personal data set (320).

Example 29: The system (1000) according to example 28, further comprising a background system (40) adapted to receive and store (106) the scatter value (430) in a database (41).

Example 30. The system according to any one of examples 28 or 29, wherein the scatter value (430) is a part of an anonymized user record (410) in the background system (40), wherein the anonymized user record (410) preferably comprises a personal identification number and a log entry.

Example 31: The system (1000) according to one of the examples 27 to 30,
wherein the capturing unit (31) of the terminal device (30) is a camera (31a), wherein the camera (31a) is adapted to capture (101) a light image (14) of the identification document (10); and
wherein the evaluation unit (33) of the terminal device (30) comprises a face recognition module (330), wherein the face recognition module (330) is adapted to analyze the captured light image (14) by computer-aided face recognition methods in order to obtain the biometric data of the personal data set (320).

Example 32: The system (1000) according to one of the examples 27 to 31,
wherein the capturing unit (31) of the terminal device (30) is a camera (31a), wherein the camera (31a) is adapted to capture (101) personal data (15) of the identification document (10); and
wherein the evaluation unit (33) of the terminal device (30) comprises a character recognition module (331), the character recognition module (331) being adapted to analyze the captured personal data (15) by optical character recognition methods in order to obtain the personal data of the personal data set (320).

Example 33: The system (1000) according to one of the examples 27 to 32,
wherein the identification document (10) comprises an electronic data storage device (11);
wherein the acquisition unit (31) of the terminal device (30) is a reading unit (31b), wherein the reading unit (31b) is adapted to read out electronic data (11) of a data memory of the identification document (10); and
wherein the evaluation unit (33) of the terminal device (30) is set up to evaluate the read-out data (11) in order to obtain the personal data set (320).

Example 34: The system (1000) according to one of the examples 27 to 33,
wherein the evaluation unit (33) comprises an encryption module (332), wherein the encryption module (332) is adapted to cryptographically secure the received personal data set (320); and
wherein the memory unit (32) of the terminal device (30) is configured to store the cryptographically secured personal data set (320).

Example 35: A computer program product comprising instructions which, when the program is executed by a computer, cause the computer to execute the units or modules of the system according to any of the preceding examples 1 to 34.

Within the scope of the invention, all described and/or drawn and/or claimed elements can be combined with each other as desired.

### REFERENCE SIGNS LIST

1 Person, user
   1a Face
   1b Hand
2 Test instance, if necessary with recording unit,
10 Identification document, ID
   11 Electronic data of a data storage device
   12 Front side
   13 Back side
   14 Photograph
   15 Personal data
20 Object for procedure, test cassette for SARS-CoV-2 test
   21 Object identification data set
      210 QR-code
      220 Serial number
   22 Shape, contour
30 Terminal device
   31 Capturing unit,
      31a Camera
      31b Reading unit
      31c Code-scanner
   32 Storage unit
      320 Personal data set
      321 Procedure-identification data set
   33 Evaluation unit, control unit, CPU
      330 Face recognition module
      331 Character recognition module
      332 Encryption module
      333 Pattern recognition module
   34 Display unit
      340 Procedure result-validation data set
      341 Instruction regarding procedure
      342 Instruction regarding correction in the procedure
      343 Warning
      344 (Interim) result
   35 Input unit
40 Background system
   41 Storage unit, database
      410 Anonymized personal data set
      420 Object identification data set
      430 Hash value via personal data set 320
      440 Validation token
      450 Procedure data set
   42 User set-up-system
   50 Aid (form, contour)
100 process steps for setting up
   1001 Advertisement AGB,
   1002 Personal input for the purpose of GTC
   1003 Check existence of person profile
   1004 Check quarantine end date
   101 Capturing data
   1011 Request to show the identification document
   1012 Request to show face
   1013 Encrypted data stream
   102 Analyzing the recorded data
   1021 Character recognition on the captured identification document
   1022 Face recognition on the photograph & on the face
   1023 Character recognition and face recognition completed
   1024 Display of an error with explanation
   1025 Completion of the user data set
   103 Saving a personal data set as a personal profile
   104 Cryptographic protection of the personal data set
   105 Generating a scatter value
   106 Sending terminal device identification data set
199 Selection
200 process steps to track
   201 Identifying the person
      2011 Collection of biometric data
      2012 Comparison with stored personal data
      2013 Querying the background system
      2014 Identification successful
   202 Identifying the object/aid
      2021 Capture of an object identification data set
      2022 Compare with object data set
      2023 Validate the object data set by querying the background system
      2024 Invalidation of the object data set
203a-k Tracking of person and object during procedure execution
   2030 Selection of a procedure type
   2031 Detection of position, movement of person, object, aid
   2032 Analyzing using computer-aided detection
204 Instructing/informing Procedure execution
   2041 Introductory video
   2042 Instructions for setting up the terminal device
   2043 Testing the terminal device setup
   2044a Prepare test for identification
      20441 Record test
      20442 Connect QR code and test cassette
   2044b Instructions for unpacking/assembling/capturing the object
   2045a-f Procedure cancellation information
   2046 Query conditions for procedure
   2047 Display waiting time
   2048a-d Instructions procedure step and recording the object
   2049a-b Display procedure result
   205 Documenting the performance of the procedure
   2100 Flowchart swab test
   2144a-c Instructions for the swab test
   206 Check whether all process steps have been carried out
300 process steps for validation
   3001 Compare personal data set with person
   301 Request validation of a procedure result
   302 Inquiry on the background system
   303 Create a validation token
   304 Transferring the validation token
   305 Display a machine-readable code with validation token
306 Reading the machine-readable code
   307a,b Checking the machine-readable code
308a-c Display of the validation result
1000 System to set up
2000 Tracking system
3000 Validation system
BI module: Biometric identification
ATK module: Authorize test kit
QR module: QR code reader
TR module for determining the test result
DWC module for determining the position/orientation of the test window
CTW module for interpreting the test result
BL module to determine whether test cassette is being processed
TKO module for determining test components in the image
PSM module for recognizing a user's pose
PD module for photo documentation
O-VAL Validation window
L-VAL Local certificate with local data
VAL Certificate
WEB web service

1. A computer-implemented method for computer-aided tracking of a procedure to be performed by a person, wherein the method comprises the following steps:
identifying the person by a terminal device immediately before the person performs the procedure with:
capturing of biometric data of the person by the terminal device; and
comparing of the captured biometric data with biometric data of a personal data set by the terminal device, wherein the personal data set is stored in a storage unit of the terminal device;
identifying at least one object to be modified by the procedure by the terminal device immediately before the procedure is carried out;
tracking at least one position of the person and at least one position of the at least one object by the terminal device by methods of computer-aided gesture and/or pattern and/or image recognition during the performance of the procedure;
instructing the person to perform the procedure by the terminal device during the performance of the procedure; and
documenting the performance of the procedure by capturing images at one or more random times during the performance of the procedure by a camera of the terminal device and storing this procedure documentation locally in the terminal device in a procedure-identification data set.

2. The method according to claim 1, wherein for identifying the person in the capturing step, a face of the person is captured by a capturing unit, in particular a camera of the terminal device, and the captured face is analyzed by methods of computer-aided face recognition by an evaluation unit of the terminal device, in order to obtain the biometric data of the person, wherein preferably the identification of the person is repeated at one or more random times during the performance of the procedure.

3. The method according to claim 1, wherein the procedure is a human medical examination of a body fluid of the person himself or a veterinary medical examination of a body fluid on an animal by the person, wherein the body fluid is blood, swab, saliva, aerosol, tear fluid, sweat, sputum, urine, a collection from a skin surface and/or feces of the person and/or the animal.

4. The method according to claim 1, wherein the identifying of the object comprises:
capturing an object identification data set by the terminal device; and
comparing the captured object identification data set with an object identification data set or a procedure data set of a background system.

5. The method according to claim 1, wherein immediately before the person performs the procedure, all aids to be used during the procedure are identified by the terminal device, wherein preferably each used aid is captured by a camera of the terminal device and the captured aid is analyzed by methods of computer-aided face and/or pattern and/or image recognition by an evaluation unit of the terminal device in order to identify the aid.

6. The method according to claim 1, wherein for identifying the object in the capturing step, a machine-readable data set is captured by a capturing unit, in particular a camera, of the terminal device and the machine-readable data set of the object is validated by querying a corresponding object identification data set in a background system, wherein preferably after validation of the machine-readable data set the object identification data set or the procedure data set in a database of the background system becomes invalid.

7. The method according to claim 1, wherein in the tracking step the at least one position and/or a movement of the person, preferably also a movement of a body part of the person, and the at least one position and/or a movement of the object is detected by a camera of the terminal device and the detected position and movement of the person and of the object is analyzed by methods of computer-aided face and/or pattern and/or image recognition by an evaluation unit of the terminal device.

8. The method according to claim 1, wherein, in the tracking step, an object property of the object is detected by a camera of the terminal device and the detected object property of the object is analyzed by methods of computer-aided face and/or pattern and/or image recognition by an evaluation unit of the terminal device and the analyzed object property of the object is compared with an object property to be expected during the procedure.

9. The method according to claim 8, wherein the method is terminated with an error message if it is determined in the analyzing step that the detected position of the person and/or the detected movement of the person and/or the detected position of the object and/or the detected movement of the object are outside fixed specifications of the procedure.

10. The method according to claim 1, wherein a procedure data set of a background system together with the procedure identification data set maps a procedure result.

11. The method according to claim 1, wherein for the instructing instructional indications are displayed on a display unit of the terminal device, wherein an instructional indication is provided in dependence on the performance of the procedure and/or in dependence on the tracking of positions and movements of the person and the object by the terminal device during the performance of the procedure.

12. A system for computer-aided tracking of a procedure to be performed by a person, wherein the system comprises:
an identification unit of a terminal device adapted to identify the person immediately before the person performs the procedure, the identification unit comprising:
a capturing unit of the terminal device, adapted to capturing of biometric data of the person; and
an evaluation unit of the terminal device, adapted to comparing the captured biometric data with biometric data of a personal data set, wherein the personal data set is stored in a storage unit of the terminal device,
wherein the identification unit is further adapted to identify an object to be modified by the procedure immediately before the procedure is performed;
a tracking unit of the terminal device adapted to track at least a position of the person and at least a position of the object by the terminal device by methods of computer-aided gesture and/or pattern and/or image recognition during the performance of the procedure; and
a display unit of the terminal device, adapted to instruct the person to perform the procedure during the performance of the procedure,
wherein the performance of the procedure is documented by capturing images at one or more random times during the performance of the procedure by a camera of the terminal device and this procedure documentation is stored locally in the terminal device in a procedure identification data set.

13. A computer-implemented method for computer-aided validation of a procedure performed by a person and tracked by a computer, wherein the method comprises the following steps:
requesting a validation of a procedure result by a person input at a terminal device;
generating and sending a validation request at a background system comprising a scatter value over the personal data set and a procedure identification data set of the procedure result to be validated;
receiving in the terminal device a validation token from the background system;
displaying a machine-readable code using the validation token and displaying the result of the procedure on a display unit of the terminal device;
reading of the machine-readable code by a test instance and checking of the validation token; and
displaying of a validation result relating to the procedure result together with the procedure result and the scatter value via the personal data set and preferably procedure information of the background system on a display unit of the test instance.

14. The method according to claim 13, wherein the computer-aided tracked procedure according to a method for computer-aided tracking a procedure to be performed by a person was computer-aided tracked.

15. A computer program product comprising instructions which, when the program is executed by a computer, cause the computer to perform the steps of the method according to claim 14.

16. A computer program product comprising instructions which, when the program is executed by a computer, cause the computer to perform the steps of the method according to claim 13.

17. A computer program product comprising instructions which, when the program is executed by a computer, cause the computer to perform the steps of the method according to claim 1.

18. The method according to claim 7, wherein the method is terminated with an error message if it is determined in the analyzing step that the detected position of the person and/or the detected movement of the person and/or the detected position of the object and/or the detected movement of the object are outside fixed specifications of the procedure.
